SEQUENCE LISTING (116 sequences)

SEQ ID NO: 1 - DNA, 27 bp, Artificial Sequence
  Oligonucleotide for amplifying region A
agtggaatcgaatcgttatgctgaaca

SEQ ID NO: 2 - DNA, 22 bp, Artificial Sequence
  Oligonucleotide for amplifying region A
ggacagagacacaaaattgggg

SEQ ID NO: 3 - DNA, 21 bp, Artificial Sequence
  Oligonucleotide for amplifying region A
cgtcgtatacatagtggaatc

SEQ ID NO: 4 - DNA, 20 bp, Artificial Sequence
  Oligonucleotide for amplifying region A
ymymttatatcccgaraara

SEQ ID NO: 5 - DNA, 20 bp, Artificial Sequence
  Oligonucleotide for amplifying region B
ttccatacccattgaccatc

SEQ ID NO: 6 - DNA, 20 bp, Artificial Sequence
  Oligonucleotide for amplifying region B
aaaggcgaagaagtacaagg

SEQ ID NO: 7 - DNA, 19 bp, Artificial Sequence
  Oligonucleotide for amplifying region C
cgtcgcaacgaaagagwcg

SEQ ID NO: 8 - DNA, 20 bp, Artificial Sequence
  Oligonucleotide for amplifying region C
cccacgctaatttatagtcg

SEQ ID NO: 9 - DNA, 63 bp, Haemonchus contortus
  misc_feature (4)..(4): n is a, c, g, or t
tttngacttgatgttttgttaactgctgttatatcgccgcagtacgcgtaaggctgattantg

SEQ ID NO: 10 - protein, 14 aa, Artificial Sequence
  Deduced amino acid sequence in frame 1
LDVLLTAVISPQYA

SEQ ID NO: 11 - protein, 6 aa, Artificial Sequence
  Deduced amino acid sequence of frame 2
FDLMFC

SEQ ID NO: 12 - protein, 17 aa, Artificial Sequence
  Deduced amino acid sequence of frame 3
CFVNCCYIAAVRVRLIT

SEQ ID NO: 13 - protein, 12 aa, Artificial Sequence
  Deduced amino acid sequence of reverse frame 1
SALRVLRRYNSS

SEQ ID NO: 14 - protein, 20 aa, Artificial Sequence
  Deduced amino acid sequence of reverse frame 2
SNQPYAYCGDITAVNKTSSQ

SEQ ID NO: 15 - protein, 10 aa, Artificial Sequence
  Deduced amino acid sequence of reverse frame 3
VISLTRTAAI

SEQ ID NO: 16 - DNA, 20 bp, Artificial Sequence
  Oligonucleotide
accttacctatacacccgtc

SEQ ID NO: 17 - DNA, 21 bp, Artificial Sequence
  Oligonucleotide
cttgccgttattacaccctcg

SEQ ID NO: 18 - DNA, 739 bp, Haemonchus contortus
ggaatcgaatcgttatgctgaacaactatatgaggatctcctttactattataataaaaatgtgcgaccggtaaagaatgccagtgaaagtatcaaagtgaaatttggagcctcgcttatccgtattatagacgtggtgggtgttttcaaatgctattttttagtggattcgcaccacttcttccgaattctttacgaggtttcaagctcttttggtaattatgtatataacctcaacctcaatattaagtttgttgcaaaattgataatacatgaagtgcttgaacctcactttgtcctcaaaaatcgatgacgcagaaattattgatgctattctgacccagaatggtttgacgatttgagagctctggttttctttgaaaaggcgtgacaccaagtggtcgcagagatgagttgttttccagaacttcggttcgtgctcattacaatgtattgagatgtgagcagctcaacgatccagccttctgtgacacccgaattgacagcttccagttcctactatgataattttgttgaagtgttatcgcttcatttacaagtattgggttctacgacacaatgtgagcaccatatagaaaagaacggaaggcaataaaacgattctccgctataatttaatgtaaatagaatgtcatcaagcgcgaagatgacgagttcgtgagagacgatcacaatgatcttctctggtcgtctgatgataacaacgacttatctttcg

SEQ ID NO: 19 - DNA, 697 bp, Haemonchus contortus
gtggaatcgaatcgttatgctgaacaactatatgaggatctcctttactattataataaaaatgtgcgaccggtaaagaatgccagtgaaagtatcaaagtgaaatttggagcctcgcttatccgtattatagacgtggtgggtgttttcaaatgctattttttagtggattcgcaccacttcttccgaattctttacgaggtttcaagctcttttggtaattatgtatataacctcaacctcaatattaagtttgttgcaaaattgataatacatgaagtgcttgaacctcactttgtcctcaaaaatcgatgacgcagaaattattgatgctattctgacccagaatggtttgacgatttgagagctctggttttctttgaaaaggcgtgacaccaagtggtcgcagagatgagttgttttccagaacttcggttcgtgctcattacaatgtattgagatgtgagcagctcaacgatccagccttctgtgacacccgaattgacagcttccagttcctactatgataattttgttgaagtgttatcgcttcatttacaagtattgggttctacgacacaatgtgagcaccatatagaaaagaacggaaggcaataaaacgattctccgctataatttaatgtaaatagaatgtcatcaagcgcgaagatgacgagttcgtgagagacgatcacaa

SEQ ID NO: 20 - DNA, 739 bp, Haemonchus contortus; identical to SEQ ID NO: 18

SEQ ID NO: 21 - DNA, 740 bp, Haemonchus contortus
ggaatcgaatcgttatgctgaacaactatatgaggatctcctttactattataataaaaatgtgcgaccggtaaagaatgccagtgaaagtatcaaagtgaaatttggagcctcgcttatccgtattatagacgtggtgggtgttttcaaatgctattttttagtggattcgcaccacttcttccgaattctttacgaggtttcaagctcttttggtaattatgtatataacctcaacctcaatattaagtttgttgcaaaattgataatacatgaagtgcttgaacctcactttgtcctcaaaaatcgatgacgcagaaattattgatgctattctgacccagaatggtttgacgatttgagagctctggttttctttgaaaaggcgtgacaccaagtggtcgcagagatgagttgttttccagaacttcggttcgtgctcattacaatgtattgagatgtgagcagctcaacgatccagccttctgtgacacccgaattgacagcttccagttcctactatgataattttgttgaagtgttatcgcttcatttacaagtattgggttctacgacacaatgtgagcaccatatagaaaagaacggaaggcaataaaacgattctccgctataatttaatgtaaatagaatgtcatcaagcgcgaagatgacgagttcgtgagagacgatcacaatgatcttctctggtcgtctgatgataacaacgacttatcttttcg

SEQ ID NO: 22 - DNA, 731 bp, Haemonchus contortus
agtggaatcgaatcgttatgctgaacaactatatgaggatctcctttactattataataaaaatgtgcgaccggtaaagaatgccagtgaaagtatcaaagtgaaattcggagcctcgcttatccgtatcatagacgtggtgggtattttcaaattcttattttccggtgaattcgtgccagttttttccgagctatttttttgagctttcctggccattatgtaatccgaacttcagtattaagtttattgcaagaatccataattcatgaagtgcctgggcctttcttcgcctgcaaaaatcggtgccgcagaatttattggtgctattttaaccaagaacggtttgaggactggagagctttaacttccctcgaaaaggcctgacaccaagtggtcgcaaacataagtttctttccagatcttcggttcgtgctcattaaaatgaattgaggtgtgagcagctcaacgactcagcctcctgtaacacccatatcggtagcttccggctcttactatgacaattttgttgaggtgttatcgctttacttccatgtgttgggtttctgcgacatgtgagcaccatctaaaaaggaacggaaggcaataaaacgattctccactctgatgcaatgtaaaagagaaggttatcaagcgcgacgatgacgagtccgtgagagacgatcccagtgatcttttcggtcatctgatgataactacgtatcttctcg

SEQ ID NO: 23 - DNA, 738 bp, Haemonchus contortus
gtggaatcgaatcgttatgctgaacaactctatgaggatctcctttactattataataaaaatgtgcgaccggtaaagaatgccagtgaaagtatcaaagtgaaattcggagcctcgcttatccgtattatagacgtggtgggtgttttcaaatgctatttttcagtggattcgcaccacttcttccgaattcttcacgacgtttcaagctcttttggtcattatatgacctcaacttcatgatcaagtttgttgcaaaatttgataatacatgaactgcttgagcctaactttgtccgcaaaaatcgatgccgcagaaattattgatgctattctgacccagaatggtttgaggacttgagagctctggttttctttgaaaaggcgtgacaccaagtggtcgcacagatgagttgctttccagaacttcggttcgtgctcattacagtgtattgagatgtgagtacctcaacgatccagccttttgtgacacccgaattgacagcttccagttcctactatgacaattttgttgaggtgttatcgttttttttacaagtattgggttcctgcgacataatgtgggcaccatctagaaggggacggaaggcagtcaaacgattcttcaatatgatgttatgtaaaatagaaggtatcaagtgtgatgaggacgagttcgtgagagaccatcagaatgatcttttcttgtcatgtgatgataacaacgacatatcttct

SEQ ID NO: 24 - DNA, 734 bp, Haemonchus contortus
ggaatcgaatcgttatgctgaacaactctatgaggatctcctttactattataataaaaatgtgcgaccggtaaagaatgccagtgaaagtatcaaagtgaaattcggagcctcgcttatccgtattatagacgtggtgggtgttttcaaatgctatttttcagtggattcgcaccacttcttccgaattcttcacgacgtttcaagctcttttggtcattatatgacctcaacttcatgatcaagtttgttgcaaaatttgataatacatgaactgcttgagcctaactttgtccgcaaaaatcgatgccgcagaaattattgatgctattctgacccagaatggtttgaggacttgagagctctggttttctttgaaaaggcgtgacaccaagtggtcgcacagatgagttgctttccagaacttcggttcgtgctcattacagtgtattgagatgtgagtacctcaacgatccagccttttgtgacacccgaattgacagcttccagttcctactatgacaattttgttgaggtgttatcgttttttttacaagtattgggttcctgcgacataatgtgggcaccatctagaaggggacggaaggcagtcaaacgattcttcaatatgatgttatgtaaaatagaaggtatcaagtgtgatgaggacgagttcgtgagagaccatcagaatgatcttttcttgtcatgtgatgataacaacgacatatctc

SEQ ID NO: 25 - DNA, 741 bp, Haemonchus contortus
agtggaatcgaatcgttatgctgaacaactctatgaggatctcctttactattataataaaaatgtgcgaccggtaaagaatgccagtgaaagtatcaaagtgaaattcggagcctcgcttatccgtattatagacgtggtgggtgttttcaaatgctatttttcagtggattcgcaccacttcttccgaattcttcacgacgtttcaagctcttttggtcattatatgacctcaacttcatgatcaagtttgttgcaaaatttgataatacatgaactgcttgagcctaactttgtccgcaaaaatcgatgccgcagaaattattgatgctattctgacccagaatggtttgaggacttgagagctctggttttctttgaaaaggcgtgacaccaagtggtcgcacagatgagttgctttccagaacttcggttcgtgctcattacagtgtattgagatgtgagtacctcaacgatccagccttttgtgacacccgaattgacagcttccagttcctactatgacaattttgttgaggtgttatcgttttttttacaagtattgggttcctgcgacataatgtgggcaccatctagaaggggacggaaggcagtcaaacgattcttcaatatgatgttatgtaaaatagaaggtatcaagtgtgatgaggacgagttcgtgagagaccatcagaatgatcttttcttgtcatgtgatgataacaacgacatatcttctcg

SEQ ID NO: 26 - DNA, 743 bp, Haemonchus contortus
caagtggaatcgaatcgttatgccgaacaactctatgaggatctcctttactattataataaaaatgtgcgaccggtaaagaatgccagtgaaagtatcaaagtgaaatttggagcctcgcttatacgtatcatagacgtggtgggtattttcaagtgcttattttccagtgaatttgaaccagtttttcctattctttttggggattatagctcttttggtcattatgtaacctcaacttcacgatcaaacttgttgcaaaaatcgatggtacataaagtgcttggaccttacttcgccttcaaaaatcggggcagttttggcccaaaacgttttgaggacttgaaagctttagtttccttcgagaaggcgggacacgaagtcgtcgcacagatgagtttcgtccagaacttcagttcgtgctcattgcagtgagtaaagacgtgagtagctcaacgattcagtcatctgtaacactcggatcgatagcttccggttattactatgacgattttgttgaggtattatcgctttatttacaagcattgagattcttcgacacaatgtgagcaccatctagaaaggaacggaaggcaatagaacgattctccaccttgtaaaacagatcaattactatgaatacgagttcgtgagagacgaccacaataatcatccctcgtcacctgacggcaacaacgacgatggacccagcacatcgcttaatcaaccttgtgtcttttcgg

SEQ ID NO: 27 - DNA, 730 bp, Haemonchus contortus
gtggaatcgaatcgttatgctgaacaactatatgaggatctcctttactattataataaaaatgtgcgaccggtaaagaatgccagtgaaagtatcaaagtgaaattcggagcctcgcttatccgtatcatagacgtggtgggtattttcaaatgcttattttccgtggattcgtaccagttttttccgagctatttttttcgagctttcttggtcattatgtaacccgaacttcattattaagtttattgcaagaatccataattcatgaagtgcctgggcctttcttcgcctgcaaaaatcggtgccgcagaatttattggtgctattttaaccaagaacggtttgaggactggagagctttagcatccctcgaaaaggcgtgacaccaatgggtcgcaaacttaagtttctttccagatcttaagttcgtgctcattaagatgaattgaggtgtgagtagctcaacgactcagcctcctgtaacacccatatcggtagcttccggctcttactatgacaattttgttgaggtgttatcgctttacttgcatgtgttgggtttctgcgacatgtgagcaccatctaaaaaggaacggaaggcaacaaaacgattctccactctgatgcaatgtaaaagggaaggttatcaagcgcgacgatgacgagtccgtgagagacgatcccagtaatcttttcggtcatctgatgataactacatatcttttcg

SEQ ID NO: 28 - DNA, 750 bp, Haemonchus contortus
gggaatcgaatcgttatgctgaacaactctatgaggatctcctttactattataataaaaatgtgcgaccggtaaagaacgccagtgaaagtatcaaagtgaaattcggagcctcgcttatccgtatcatagacgtggtgggtattttcaaatgcttattttccagtgaattcgaaccagtttttgaaattcttttcgagtttatagctcttttggtcattatgtaacctcaacttcatgatcaaactttttgcaaaaatcgatagtacatgaagtgcttggacctcactttgcatgcaaaaataggtgtcgcagtagttattggtgctgtcttgacccagaacggtttgaggacttgagaactctggtttactttgaaaaagcgtgacaccaagtggtcgcacagataagtttttctcaggatttcagttcatgctcattgctgtgaataaagacgtgagtagcgtaacgtattttctgtaacacccagatcgatagcttccggttctactatgacaattttgttgagatattagcgctttatttacaagtttcggattattacgacacaatgtgaccatcatcttgcatggaacggaaggcattaaaacgattctccgctatgatgttatgtagaacagaagattatcaagtgcgacgaagacgatcacaatgatcctctttggacacctgatgataacaacgaagacggacccagcatatagcgagattcacccttgaatcttctcg

SEQ ID NO: 29 - DNA, 750 bp, Haemonchus contortus
tatgctgaacaactctatgaggatctcctttactattataataaaaatgtgcgaccggtaaagaacgccagtgaaagtatcaaagtgaaattcggagcctcgcttatccgtatcatagacgtggtgggtattttcaaatgcttattttccagtgaattcgaaccagtttttgaaattcttttcgagtttatagctcttttggtcattatgtaacctcaacttcatgatcaaactttttgcaaaaatcgatagtacatgaagtgcttggacctcactttgcatgcaaaaataggtgtcgcagtagttattggtgctgtcttgacccagaacggtttgaggacttgagaactctggtttactttgaaaaagcgtgacaccaagtggtcgcacagataagtttttctcaggatttcagttcatgctcattgctgtgaataaagacgtgagtagcgtaacgtattttctgtaacacccagatcgatagcttccggttctactatgacaattttgttgagatattagcgctttatttacaagtttcggattattacgacacaatgtgaccatcatcttgcatggaacggaaggcattaaaacgattctccgctatgatgttatgtagaacagaagattatcaagtgcgacgaagacgatcacaatgatcctctttggacacctgatgataacaacgaagacggacccagcatatagcgagattcacccttgaatcttctcgggatatagaggttc

SEQ ID NO: 30 - DNA, 775 bp, Haemonchus contortus
tatgctgaacaactctatgaggatctcctttactattataataaaaatgtgcgaccggtaaagaacgccagtgaaagtatcaaagtgaaattcggagcctcgcttatccgtatcatagacgtggtgggtattttcaaatgcttattttccagtgaattcgaaccagtttttgaaattcttttcgagtttatagctcttttggtcattatgtaacctcaacttcatgatcaaactttttgcaaaaatcgatagtacatgaagtgcttggacctcactttgcatgcaaaaataggtgtcgcagtagttattggtgctgtcttgacccagaacggtttgaggacttgagaactctggtttactttgaaaaagcgtgacaccaagtggtcgcacagataagtttttctcaggatttcagttcatgctcattgctgtgaataaagacgtgagtagcgtaacgtattttctgtaacacccagatcgatagcttccggttctactatgacaattttgttgagatattagcgctttatttacaagtttcggattattacgacacaatgtgaccatcatcttgcatggaacggaaggcattaaaacgattctccgctatgatgttatgtagaacagaagattatcaagtgcgacgaagacgatcacaatgatcctctttggacacctgatgataacaacgaagacggacccagcatatagcgagattcacccttgaatcttctcgggatatagaggttccaaagtgaagcgtagcccccaattg

SEQ ID NO: 31 - DNA, 140 bp, Haemonchus contortus
taaaaatgtgcgaccggtaaagaacgccagtgaaagtatcaaagtgaaattcggagcctcgcttatccgtatcatagacgtggtgggtattttcaaatgcttattttccagtgaattcgaaccagtttttgaaattcttt

SEQ ID NO: 32 - DNA, 140 bp, Haemonchus contortus; identical to SEQ ID NO: 31

SEQ ID NO: 33 - DNA, 805 bp, Haemonchus contortus
gtggaaattcgaatcgttatgctgaacaactctatgaggatctcctttactattataataaaaatgtgcgaccggtaaagaacgccagtgaaagtatcaaagtgaaattcggagcctcgcttatccgtatcatagacgtggtgggtattttcaaatgcttattttccagtgaattcgaaccagtttttgaaattcttttcgagtttatagctcttttggtcattatgtaacctcaacttcatgatcaaactttttgcaaaaatcgatagtacatgaagtgcttggacctcactttgcatgcaaaaataggtgtcgcagtagttattggtgctgtcttgacccagaacggtttgaggacttgagaactctggtttactttgaaaaagcgtgacaccaagtggtcgcacagataagtttttctcaggatttcagttcatgctcattgctgtgaataaagacgtgagtagcgtaacgtattttctgtaacacccagatcgatagcttccggttctactatgacaattttgttgagatattagcgctttatttacaagtttcggattattacgacacaatgtgaccatcatcttgcatggaacggaaggcattaaaacgattctccgctatgatgttatgtagaacagaagattatcaagtgcgacgaagacgatcacaatgatcctctttggacacctgatgataacaacgaagacggacccagcatatagcgagattcacccttgaatcttctcgggatatagaggttccaaagtgaagcgtagcccccaattttgtgtctctgtcc

SEQ ID NO: 34 - DNA, 749 bp, Haemonchus contortus
ggaatcgaatcgttatgctgaacaactctatgaggatctcctttactattataataaaaatgtgcgaccggtaaagaacgccagtgaaagtatcaaagtgaaattcggagcctcgcttatccgtatcatagacgtggtgggtattttcaaatgcttattttccagtgaattcgaaccagtttttgaaattcttttcgagtttatagctcttttggtcattatgtaacctcaacttcatgatcaaactttttgcaaaaatcgatagtacatgaagtgcttggacctcactttgcatgcaaaaataggtgtcgcagtagttattggtgctgtcttgacccagaacggtttgaggacttgagaactctggtttactttgaaaaagcgtgacaccaagtggtcgcacagataagtttttctcaggatttcagttcatgctcattgctgtgaataaagacgtgagtagcgtaacgtattttctgtaacacccagatcgatagcttccggttctactatgacaattttgttgagatattagcgctttatttacaagtttcggattattacgacacaatgtgaccatcatcttgcatggaacggaaggcattaaaacgattctccgctatgatgttatgtagaacagaagattatcaagtgcgacgaagacgatcacaatgatcctctttggacacctgatgataacaacgaagacggacccagcatatagcgagattcacccttgaatcttctcg

SEQ ID NO: 35 - DNA, 748 bp, Haemonchus contortus
ggaatcgaatcgttatgctgaacaactctatgaggatctcctttactattataataaaaatgtgcgaccggtaaagaacgccagtgaaagtatcaaagtgaaattcggagcctcgcttatccgtatcatagacgtggtgggtattttcaaatgcttattttccagtgaattcgaaccagtttttgaaattcttttcgagtttatagctcttttggtcattatgtaacctcaacttcatgatcaaactttttgcaaaaatcgatagtacatgaagtgcttggacctcactttgcatgcaaaaataggtgtcgcagtagttattggtgctgtcttgacccagaacggtttgaggacttgagaactctggtttactttgaaaaagcgtgacaccaagtggtcgcacagataagtttttctcaggatttcagttcatgctcattgctgtgaataaagacgtgagtagcgtaacgtattttctgtaacacccagatcgatagcttccggttctactatgacaattttgttgagatattagcgctttatttacaagtttcggattattacgacacaatgtgaccatcatcttgcatggaacggaaggcattaaaacgattctccgctatgatgttatgtagaacagaagattatcaagtgcgacgaagacgatcacaatgatcctctttggacacctgatgataacaacgaagacggacccagcatatagcgagattcacccttgaatcttctc

SEQ ID NO: 36 - DNA, 717 bp, Haemonchus contortus
gtatgctgaacaactctatgaggatctcctctactattataataaaaatgtgcgaccggtaaagaatgccagtgaaagtatcaaagtgaaattcggagcctcgcttatccgtatcatagacgtggtgggtattttcaaattcttattttccggtgaattcgtgccagttttttccgagctatttttttgagctttcctggtcattatgtaatccgaacttcagtattaagtttattgcaagaatccataattcatgaagccgcagaatttattggtgctattttaaccgagaacggtttgaggactggagagctttaacttcccgcgaaaaggcctgacaccaagtggtcgcaaacataagtttctttccagatcttcggttcgtgctcattaaaatgaattgaggtgtgagcagctcaacgactcagcctcctgtaacacccatatcggtagcttccggctcttactatgacaattttgttgaggtgttatcgctttacttgcatgtgttgggtttctgcgacatgtgagcaccatctaaaaagaaacggaaggcaataaaacgattctccactctgatgcaatgtaaaagagaagattatcaagcgcgacgatgacgagtccgtgagagatgatcccaaagatcttttcggtcatctgatgataactacatatcttttcgggatatagagggttcaaagtgaggcgtagtcccca

SEQ ID NO: 37 - DNA, 714 bp, Haemonchus contortus
tgctgaacaactatatgaggatctcctttactattataataaaaatgtgcgaccggtaaagaatgccagtgaaagtatcaaagtgaaattcggagcctcgcttatccgtatcatagacgtggtgggtattttcaaattcttattttccggtgaattcgtgccagttttttccgagctatttttttgagctttcctggtcattatgtaatccgaacttcagtattaagtttattgcaagaatccataattcatgaagccgcagaatttattggtgctattttaaccgagaacggtttgaggactggagagctttaacttcccgcgaaaaggcctgacaccaagtggtcgcaaacataagtttctttccagatcttcggttcgtgctcattaaaatgaattgaggtgtgagcagctcaacgactcagcctcctgtaacacccatatcggtagcttccggctcttactatgacaattttgttgaggtgttatcgctttacttgcatgtgttgggtttctgcgacatgtgagcaccatctaaaaagaaacggaaggcaataaaacgattctccactctgatgcaatgtaaaagagaagattatcaagcgcgacgatgacgagtccgtgagagatgatcccaaagatcttttcggtcatctgatgataactacatatctttcgggatatagagggttcaaagtgaggcgtagtccccaa

SEQ ID NO: 38 - DNA, 721 bp, Haemonchus contortus
gtatgctgaacaactctatgaggatctcctctactattataataaaaatgtgcgaccggtaaagaatgccagtgaaagtatcaaagtgaaattcggagcctcgcttatccgtatcatagacgtggtgggtattttcaaattcttattttccggtgaattcgtgccagttttttccgagctatttttttgagctttcctggtcattatgtaatccgaacttcagtattaagtttattgcaagaatccataattcatgaagccgcagaatttattggtgctattttaaccgagaacggtttgaggactggagagctttaacttcccgcgaaaaggcctgacaccaagtggtcgcaaacataagtttctttccagatcttcggttcgtgctcattaaaatgaattgaggtgtgagcagctcaacgactcagcctcctgtaacacccatatcggtagcttccggctcttactatgacaattttgttgaggtgttatcgctttacttgcatgtgttgggtttctgcgacatgtgagcaccatctaaaaagaaacggaaggcaataaaacgattctccactctgatgcaatgtaaaagagaagattatcaagcgcgacgatgacgagtccgtgagagatgatcccaaagatcttttcggtcatctgatgataactacatatcttttcgggatatagagggttcaaagtgaggcgtagtccccaattg

SEQ ID NO: 39 - DNA, 750 bp, Haemonchus contortus
gtggaattcgaatcgttatgctgaacaactatatgaggatctcctttactattataataaaaatgtgcgaccggtaaagaatgccagtgaaagtatcaaagtgaaattcggagcctcgcttatccgtatcatagacgtggtgggtattttcaaattcttattttccggtgaattcgtgccagttttttccgagctatttttttgagctttcctggtcattatgtaatccgaacttcagtattaagtttattgcaagaatccataattcatgaagccgcagaatttattggtgctattttaaccgagaacggtttgaggactggagagctttaacttcccgcgaaaaggcctgacaccaagtggtcgcaaacataagtttctttccagatcttcggttcgtgctcattaaaatgaattgaggtgtgagcagctcaacgactcagcctcctgtaacacccatatcggtagcttccggctcttactatgacaattttgttgaggtgttatcgctttacttgcatgtgttgggtttctgcgacatgtgagcaccatctaaaaagaaacggaaggcaataaaacgattctccactctgatgcaatgtaaaagagaagattatcaagcgcgacgatgacgagtccgtgagagatgatcccaaagatcttttcggtcatctgatgataactacatatcttttcgggatatagagggttcaaagtgaggcgtagtccccaattttgtgtctctgtcca

SEQ ID NO: 40 - DNA, 750 bp, Haemonchus contortus
tggaattcgaatcgttatgctgaacaactatatgaggatctcctttactattataataaaaatgtgcgaccggtaaagaatgccagtgaaagtatcaaagtgaaattcggagcctcgcttatccgtatcatagacgtggtgggtattttcaaattcttattttccggtgaattcgtgccagttttttccgagctatttttttgagctttcctggtcattatgtaatccgaacttcagtattaagtttattgcaagaatccataattcatgaagccgcagaatttattggtgctattttaaccgagaacggtttgaggactggagagctttaacttcccgcgaaaaggcctgacaccaagtggtcgcaaacataagtttctttccagatcttcggttcgtgctcattaaaatgaattgaggtgtgagcagctcaacgactcagcctcctgtaacacccatatcggtagcttccggctcttactatgacaattttgttgaggtgttatcgctttacttgcatgtgttgggtttctgcgacatgtgagcaccatctaaaaagaaacggaaggcaataaaacgattctccactctgatgcaatgtaaaagagaagattatcaagcgcgacgatgacgagtccgtgagagatgatcccaaagatcttttcggtcatctgatgataactacatatcttttcgggatatagagggttcaaagtgaggcgtagtccccaattttgtgtctctgtccaa

SEQ ID NO: 41 - DNA, 696 bp, Haemonchus contortus
gtggaatcgaatcgttatgctgaacaactatatgaggatctcctttactattataataaaaatgtgcgaccggtaaagaatgccagtgaaagtatcaaagtgaaattcggagcctcgcttatccgtatcatagacgtggtgggtattttcaaattcttattttccggtgaattcgtgccagttttttccgagctatttttttgagctttcctggtcattatgtaatccgaacttcagtattaagtttattgcaagaatccataattcatgaagccgcagaatttattggtgctattttaaccgagaacggtttgaggactggagagctttaacttcccgcgaaaaggcctgacaccaagtggtcgcaaacataagtttctttccagatcttcggttcgtgctcattaaaatgaattgaggtgtgagcagctcaacgactcagcctcctgtaacacccatatcggtagcttccggctcttactatgacaattttgttgaggtgttatcgctttacttgcatgtgttgggtttctgcgacatgtgagcaccatctaaaaagaaacggaaggcaataaaacgattctccactctgatgcaatgtaaaagagaagattatcaagcgcgacgatgacgagtccgtgagagatgatcccaaagatcttttcggtcatctgatgataactacatatcttctcg

SEQ ID NO: 42 - DNA, 703 bp, Haemonchus contortus
catagtggaatcgaatcgttatgctgaacaactatatgaggatctcctttactattataataaaaatgtgcgaccggtaaagaatgccagtgaaagtatcaaagtgaaattcggagcctcgcttatccgtatcatagacgtggtgggtattttcaaattcttattttccggtgaattcgtgccagttttttccgagctatttttttgagctttcctggtcattatgtaatccgaacttcagtattaagtttattgcaagaatccataattcatgaagccgcagaatttattggtgctattttaaccgagaacggtttgaggactggagagctttaacttcccgcgaaaaggcctgacaccaagtggtcgcaaacataagtttctttccagatcttcggttcgtgctcattaaaatgaattgaggtgtgagcagctcaacgactcagcctcctgtaacacccatatcggtagcttccggctcttactatgacaattttgttgaggtgttatcgctttacttgcatgtgttgggtttctgcgacatgtgagcaccatctaaaaagaaacggaaggcaataaaacgattctccactctgatgcaatgtaaaagagaagattatcaagcgcgacgatgacgagtccgtgagagatgatcccaaagatcttttcggtcatctgatgataactacatatcttctcggga

SEQ ID NO: 43 - DNA, 695 bp, Haemonchus contortus
tggaatcgaatcgttatgctgaacaactatatgaggatctcctttactattataataaaaatgtgcgaccggtaaagaatgccagtgaaagtatcaaagtgaaattcggagcctcgcttatccgtatcatagacgtggtgggtattttcaaattcttattttccggtgaattcgtgccagttttttccgagctatttttttgagctttcctggtcattatgtaatccgaacttcagtattaagtttattgcaagaatccataattcatgaagccgcagaatttattggtgctattttaaccgagaacggtttgaggactggagagctttaacttcccgcgaaaaggcctgacaccaagtggtcgcaaacataagtttctttccagatcttcggttcgtgctcattaaaatgaattgaggtgtgagcagctcaacgactcagcctcctgtaacacccatatcggtagcttccggctcttactatgacaattttgttgaggtgttatcgctttacttgcatgtgttgggtttctgcgacatgtgagcaccatctaaaaagaaacggaaggcaataaaacgattctccactctgatgcaatgtaaaagagaagattatcaagcgcgacgatgacgagtccgtgagagatgatcccaaagatcttttcggtcatctgatgataactacatatcttctcg

SEQ ID NO: 44 - DNA, 712 bp, Haemonchus contortus
acaactatatgaggatctcctttactattataataaaaatgtgcgaccggtaaagaatgccagtgaaagtatcaaagtgaaattcggagcctcgcttrtccgtatcatagacgtggtgggtattttcaaattcytattttccggtgaattcgtgccagttttttccgagctatttttttgagctttcctggtcattatgtaatccgaacttcagtattaagtttattgcaagaatccataattcatgaagccgcagaatttattggtgctattttaaccgagaacggtttgaggactggagagctttaacttcccgcgaaaaggcctgacaccaagtggtcgcaaacataagtttctttccagatcttcggttcgtgctcattaaaatgaattgaggtgtgagcagctcaacgactcagcctcctgtaacacccatatcggtagcttccggctcttactatgacaattttgttgaggtgttatcgctttacttgcatgtgttgggtttctgcgacatgtgagcaccatctaaaaagaaacggaaggcaataaaacgattctccactctgatgcaatgtaaaagagaagattatcaagcgcgacgatgacgagtccgtgagagatgatcccaaagatcttttcggtcatctgatgataactacatatcttttcgggatatagagggttcaaagtgaggcgtagtccccaattt

SEQ ID NO: 45 - DNA, 719 bp, Haemonchus contortus
tgctgaacaactatatgaggatctcctttactattataataaaaatgtgcgaccggtaaagaatgccagtgaaagtatcaaagtgaaattcggagcctcgcttatccgtatcatagacgtggtgggtattttcaaattcttattttccggtgaattcgtgccagttttttccgagctatttttttgagctttcctggtcattatgtaatccgaacttcagtattaagtttattgcaagaatccatarttcatgaagccgcagaatttattggtgctattttaaccgagaacggtttgaggactggagagctttaacttcccgcgaaaaggcctgacaccaagtggtcgcaaacataagtttctttccagatcttcggttcgtgctcattaaaatgaattgaggtgtgagcagctcaacgactcagcctcctgtaacacccatatcggtagcttccggctcttactatgacaattttgttgaggtgttatcgctttacttgcatgtgttgggtttctgcgacatgtgagcaccatctaaaaagaaacggaaggcaataaaacgattctccactctgatgcaatgtaaaagagaagattatcaagcgcgacgatgacgagtccgtgagagatgatcccaaagatcttttcggtcatctgatgataactacatatcttttcgggatatagagggttcaaagtgaggcgtagtccccaatttg

SEQ ID NO: 46 - DNA, 878 bp, Haemonchus contortus
ccagttatactcttttctgtgggaagaaacattccaacggcatcgaggattcattgtggcggataactgccctaattactgtattgacgaatgctctattttatttcataagttgagatttttgagggaaatatcattttgagcagtcatttacgcagcttctatttccattacagttacaatcgtcgtatacatagtggaatcgaatcgttatgctgaacaactctatgaggatctcctttactattataataaaaatgtgcgaccggtaaagaacgccagtgaaagtatcaaagtgaaattcggagcctcgcttatccgtatcatagacgtggtgggtattttcaaatgcttattytccagtgaattcgaaccagtttttgaaattcttttcgagtttatagctcttttggtcattatgtaacctcaacttcatgatcaaactttttgcaaaaatcgatagtacatgaagtgcttggacctcactttgcatgcaaaaataggtgtcgcagtagttattggtgctgtcttgacccagaacggtttgaggacttgagaactctggtttactttgaaaaagcgtgacaccaagtggtcgcacagataagtttttctcaggatttcagttcatgctcattgctgtgaataaagacgtgagtagcgtaacgtattttctgtaacacccagatcgatagcttccggttctactatgacaattttgttgagatattagcgctttatttacaagtttcggattattacgacacaatgtgaccatcatcttgcatggaacggaaggcattaaaacgattctccgctatgatgttatgtagaacagaagattatcaagtgcgacgaagacgatcacaatgatctct

SEQ ID NO: 47 - DNA, 872 bp, Haemonchus contortus
cagtatactcttttctgtgggaagaaacattccaacggcatcgaggattcattgtggcggataactgccctaattactgtattgacgaatgctctattttatttcataagttgagatttttgagggaaatatcattttgagcagtcatttacgcagcttctatttccattacagttacaatcgtcgtatacatagtggaatcgaatcgttatgctgaacaactctatgaggatctcctttactattataataaaaatgtgcgaccggtaaagaacgccagtgaaagtatcaaagtgaaattcggagcctcgcttatccgtatcatagacgtggtgggtattttcaaatgcttattttccagtgaattcgaaccagtttttgaaattcttttcgagtttatagctcttttggtcattatgtaacctcaacttcatgatcaaactttttgcaaaaatcgatagtacatgaagtgcttggacctcactttgcatgcaaaaataggtgtcgcagtagttattggtgctgtcttgacccagaacggtttgaggacttgagaactctggtttactttgaaaaagcgtgacaccaagtggtcgcacagataagtttttctcaggatttcagttcatgctcattgctgtgaataaagacgtgagtagcgtaacgtattttctgtaacacccagatcgatagcttccggttctactatgacaattttgttgagatattagcgctttatttacaagtttcggattattacgacacaatgtgaccatcatcttgcatggaacggaaggcattaaaacgattctccgctatgatgttatgtagaacagaagattatcaagtgcgacgaagacgatcacatgatc

SEQ ID NO: 48 - DNA, 883 bp, Haemonchus contortus
tgtccagttatactcttttctgtgggaagaaacattccaacggcatcgaggattcattgtggcggataactgccctaattactgtattgacgaatgctctattttatttcataagttgagatttttgagggaaatatcattttgagcagtcatttacgcagcttctatttccattacagttacaatcgtcgtatacatagtggaatcgaatcgttatgctgaacaactctatgaggatctcctttactattataataaaaatgtgcgaccggtaaagaacgccagtgaaagtatcaaagtgaaattcggagcctcgcttatccgtatcatagacgtggtgggtattttcaaatgcttattttccagtgaattcgaaccagtttttgaaattcttttcgagtttatagctcttttggtcattatgtaacctcaacttcatgatcaaactttttgcaaaaatcgatagtacatgaagtgcttggacctcactttgcatgcaaaaataggtgtcgcagtagttattggtgctgtcttgacccagaacggtttgaggacttgagaactctggtttactttgaaaaagcgtgacaccaagtggtcgcacagataagtttttctcaggatttcagttcatgctcattgctgtgaataaagacgtgagtagcgtaacgtattttctgtaacacccagatcgatagcttccggttctactatgacaattttgttgagatattagcgctttatttacaagtttcggattattacgacacaatgtgaccatcatcttgcatggaacggaaggcattaaaacgattctccgctatgatgttatgtagaacagaagattatcaagtgcgacgaagacgatcacaatgatcctctg

SEQ ID NO: 49 - DNA, 883 bp, Haemonchus contortus; identical to SEQ ID NO: 48

SEQ ID NO: 50 - DNA, 692 bp, Haemonchus contortus
gtcgcgtcgcacgaaagagacgccgcgcgcgaatggatgtagcaggatgctgcaccaagcaaaaaaatacaggaacaacagaactaacattatagtcatgccaaaaataataatcgctggacgattggaaagagctgcaatatcgaggctaatggtccgcgactgccgcgcgcggcgaatcgattacacaggccgatcctattgcccatctcccactcattatcacctcatccagtcggcatggtgcagttatcgaatccctgataggtacagattcgggtcagcagaaacttccatccctgcgccatcgggtacttggtagcaggcccgtctggattatgaaagaactgaattgtacgccggaaggccactataagtcattgcatattcacattcaacctcctaacgattctgagttgaaagtgcgtccccaaaatgggattgatttttgccaagtcttcattccctactaagacgatccacgtcgaaagcactttatcaccactttgggaagtttccttaccacaccagcgttacccactgaactagtactgctgcacaacttcaatatactatatgggtataaataaaaatatatatatttcgaataaaatcatcatgaaatatttaggatgaggtgaatcaagttttgacaaccaacttatggttggagatgcaatggttcgactata

SEQ ID NO: 51 - DNA, 715 bp, Haemonchus contortus
cgggtgtggtgcagtaggaccgaaattgtcgcgtcgcaacgaaagagacgccgcgcgcgaatggatgtagcaggatgctgcaccaagcaaaaaaatacaggaacaacagaactaacattatagtcatgccaaaaataataatcgctggacgattggaaagagctgcaatatcgaggctaatggtccgcgactgccgcgcgcggcgaatcgattacacaggccgatcctattgcccatctcccactcattatcacctcatccagtcggcatggtgcagttatcgaatccctgataggtacagattcgggtcagcagaaacttccatccctgcgccatcgggtacttggtagcaggcccgtctggattatgaaagaactgaattgtacgccggaaggccactataagtcattgcatattcacattcaacctcctaacgattctgagttgaaagtgcgtccccaaaatgggattgatttttgccaagtcttcattccctactaagacgatccacgtcgaaagcactttatcaccactttgggaagtttccttaccacaccagcgttacccactgaactagtactgctgcacaacttcaatatactatatgggtataaataaaaatatatatatttcgaataaaatcatcatgaaatatttaggatgaggtgaatcaagttttgacaaccaacttatggttggagatgcaatggttcga

SEQ ID NO: 52 - DNA, 721 bp, Haemonchus contortus
tcgggtgtggtgcagtaggaccgaaattgtcgcgtcgcaacgaaagagacgccgcgcgcgaatggatgtagcaggatgctgcaccaagcaaaaaaatacaggaacaacagaactaacattatagtcatgccaaaaataataatcgctggacgattggaaagagctgcaatatcgaggctaatggtccgcgactgccgcgcgcggcgaatcgattacacaggccgatcctattgcccatctcccactcattatcacctcatccagtcggcatggtgcagttatcgaatccctgataggtacagattcgggtcagcagaaacttccatccctgcgccatcgggtacttggtagcaggcccgtctggattatgaaagaactgaattgtacgccggaaggccactataagtcattgcatattcacattcaacctcctaacgattctgagttgaaagtgcgtccccaaaatgggattgatttttgccaagtcttcattccctactaagacgatccacgtcgaaagcactttatcaccactttgggaagtttccttaccacaccagcgttacccactgaactagtactgctgcacaacttcaatatactatatgggtataaataaaaatatatatatttcgaataaaatcatcatgaaatatttaggatgaggtgaatcaagttttgacaaccaacttatggttggagatgcaatggttcgactata

SEQ ID NO: 53 - DNA, 721 bp, Haemonchus contortus; identical to SEQ ID NO: 52

SEQ ID NO: 54 - DNA, 750 bp, Haemonchus contortus
atatcgggtgtggtgtagtaggatcgaaactgcgcgtcgcaacgaaagagtcgcagcgcgcgagggaatgtagcagaatgctgcagcaagtaagaaatacgggaacaaaagaacgaacattagaatcatgccaaaaataatattctcttgtcgattgagctgtaggaagaacgctggagctgcaatatcgagatgaatggtccgcgactaccgcgtgctgcgaatcagttacacaagccgatcctactgcccacttcccactcattataacctcatccagtcgacatggtgcaattatcgaatccttgataggtataaactcgggtcagccgaatcatccatccctccgtcgtcgagtgattggtaccaagctcttctgggattaagaaagaactgaactgtacatcggaaggccatcataagtcattgcatagtcacatacatgcgcattcaacctcctaacgatgctgaatcaaaagtgcgtccctaaaacgagaccgatttttgccaagtctcgattccttactaattgtaagacgacccacatagaaaacactttgacaccactttaggaagtttcctttccacatcagcgctacccattgaactagtactgctgcgcaactacgatacaaataaataaatatatgtcatgtgtttgtcgaataaaatcatcatgaactattcaggatgaggtgaatcaggttttgacaaccaacctatggttagagatgcaatggttcgactata

SEQ ID NO: 55 - DNA, 696 bp, Haemonchus contortus
cgcagcgcgcgagggaatgtagcagaatgctgcagcaagtaagaaatacgggaacaaaagaacgaacattagaatcatgccaaaaataatattctcttgtcgattgagctgtaggaagaacgctggagctgcaatatcgagatgaatggtccgcgactaccgcgtgctgcgaatcagttacacaagccgatcctactgcccacttcccactcattataacctcatccagtcgacatggtgcaattatcgaatccttgataggtataaactcgggtcagccgaatcatccatccctccgtcgtcgagtgattggtaccaagctcttctgggattaagaaagaactgaactgtacatcggaaggccatcataagtcattgcatagtcacatacatgcgcattcaacctcctaacgatgctgaatcaaaagtgcgtccctaaaacgagaccgatttttgccaagtctcgattccttactaattgtaagacgacccacatagaaaacactttgacaccactttaggaagtttcctttccacatcagcgctacccattgaactagtactgctgcgcaactacgatacaaataaataaatatatgtcatgtgtttgtcgaataaaatcatcatgaactattcaggatgaggtgaatcaggttttgacaaccaacctatggttagagatgcaatggttcgact

SEQ ID NO: 56 - DNA, 776 bp, Haemonchus contortus
caagtgaccscaatatcgggtgtggtgtagtaggatcgaaactgcgcgtcgcaacgaaagagtcgcagcgcgcgagggaatgtagcagaatgctgcagcaagtaagaaatacgggaacaaaagaacgaacattagaatcatgccaaaaataatattctcttgtcgattgagctgtaggaagaacgctggagctgcaatatcgagatgaatggtccgcgactaccgcgtgctgcgaatcagttacacaagccgatcctactgcccacttcccactcattataacctcatccagtcgacatggtgcaattatcgaatccttgataggtataaactcgggtcagccgaatcatccatccctccgtcgtcgagtgattggtaccaagctcttctgggattaagaaagaactgaactgtacatcggaaggccatcataagtcattgcatagtcacatacatgcgcattcaacctcctaacgatgctgaatcaaaagtgcgtccctaaaacgagaccgatttttgccaagtctcgattccttactaattgtaagacgacccacatagaaaacactttgacaccactttaggaagtttcctttccacatcagcgctacccattgaactagtactgctgcgcaactacgatacaaataaataaatatatgtcatgtgtttgtcgaataaaatcatcatgaactattcaggatgaggtgaatcaggttttgacaaccaacctatggttagagatgcaatggttcgactataaattaagcgtggga

SEQ ID NO: 57 - DNA, 711 bp, Haemonchus contortus
tcaagtgaccgcaatatcgggtgtggtgtagtaggatcgaaactgcgcgtcgcaacgaaagagtcgcagcgcgcgagggaatgtagcagaatgctgcagcaagtaagaaatacgggaacaaaagaacgaacattagaatcatgccaaaaataatattctcttgtcgattgagctgtaggaagaacgctggagctgcaatatcgagatgaatggtccgcgactaccgcgtgctgcgaatcagttacacaagccgatcctactgcccacttcccactcattataacctcatccagtcgacatggtgcaattatcgaatccttgataggtataaactcgggtcagccgaatcatccatccctccgtcgtcgagtgattggtaccaagctcttctgggattaagaaagaactgaactgtacatcggaaggccatcataagtcattgcatagtcacatacatgcgcattcaacctcctaacgatgctgaatcaaaagtgcgtccctaaaacgagaccgatttttgccaagtctcgattccttactaattgtaagacgacccacatagaaaacactttgacaccactttaggaagtttcctttccacatcagcgctacccattgaactagtactgctgcgcaactacgatacaaataaataaatatatgtcatgtgtttgtcgaataaaatcatcatgaactattcaggatgaggtg

SEQ ID NO: 58 - DNA, 912 bp, Haemonchus contortus
tgtcgcgtcgcacgaaagagacgccgcgcgcgaaggagtgtagcagaatgctgcagcaagtaagaaatacagggacaaatacctaacattagaatcatgccaataattataatcgcgtgtcgattgagctggagctgcaatatcgaggcgaattgtccgcgacgggtgtgtgtcaagtcgtgcgtccgagggtgcgcgcggcaaattgcgcggcgaagttcgtggcgattgtaattggggcccaaatctgaagtaaggtagattagtgaggtcagctgatcgtactattctactgtacttcgaagttgggcccaaataataggcgctaaagccgtcagaatagttatgggtttcgccgcgcaagttgcctcggacgcacgacttgaccgtcccccgtccgcgactgccgcgcgcggtcagtcgattacacaaaccgatcctactgcccatatcccactcattatcacctcatccagtcggcatggtgcaactattgaatcccttatagtatagactcgggtcagcagaatcttccatccctgcgccgtcgggtacttggtaccaggctcgtctgggtttatgaaagaactgaattgtacattggaaggccactataagtcattgcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcacagcttcaatataaataaataaataaataaatgttatgtgtttgtgaataagactatcatgaactattcaggatgaggtgaatcaggttttgacaaccaac

SEQ ID NO: 59 - DNA, 893 bp, Haemonchus contortus
gctgcagcagtaagaaatacagggacaaatacctaacattagaatcatgccaataattataatcgcgtgtcgattgagctggagctgcaatatcgaggcgaattgtccgcgacgggtgtgtgtcaagtcgtgcgtccgagggtgcgcgcggcaaattgcgcggcgaagttcgtggcgattgtaattggggcccaaatctgaagtaaggtagattagtgaggtcagctgatcgtactattctactgtacttcgaagttgggcccaaataataggcgctaaagccgtcagaatagttatgggtttcgccgcgcaagttgcctcggacgcacgacttgaccgtcccccgtccgcgactgccgcgcgcggtcagtcgattacacaaaccgatcctactgcccatatcccactcattatcacctcatccagtcggcatggtgcaactattgaatcccttatagtatagactcgggtcagcagaatcttccatccctgcgccgtcgggtacttggtaccaggctcgtctgggtttatgaaagaactgaattgtacattggaaggccactataagtcattgcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcacagcttcaatataaataaataaataaataaatgttatgtgtttgtgaataagactatcatgaactattcaggatgaggtgaatcaggttttgacaaccaacctatggttagagatgcaatggttcgactata

SEQ ID NO: 60 - DNA, 896 bp, Haemonchus contortus
ctgcagcagtaagaaatacagggacaaatacctaacattagaatcatgccaataattataatcgcgtgtcgattgagctggagctgcaatatcgaggcgaattgtccgcgacgggtgtgtgtcaagtcgtgcgtccgagggtgcgcgcggcaaattgcgcggcgaagttcgtggcgattgtaattggggcccaaatctgaagtaaggtagattagtgaggtcagctgatcgtactattctactgtacttcgaagttgggcccaaataataggcgctaaagccgtcagaatagttatgggtttcgccgcgcaagttgcctcggacgcacgacttgaccgtcccccgtccgcgactgccgcgcgcggtcagtcgattacacaaaccgatcctactgcccatatcccactcattatcacctcatccagtcggcatggtgcaactattgaatcccttatagtatagactcgggtcagcagaatcttccatccctgcgccgtcgggtacttggtaccaggctcgtctgggtttatgaaagaactgaattgtacattggaaggccactataagtcattgcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcacagcttcaatataaataaataaataaataaatgttatgtgtttgtgaataagactatcatgaactattcaggatgaggtgaatcaggttttgacaaccaacctatgggttagagatgcaatggttcgactataatt

SEQ ID NO: 61 - DNA, 887 bp, Haemonchus contortus
cgccgcgcgcgaaggagtgtagcagaatgctgcagcaagtaagaaatacagggacaaatacctaacattagaatcatgccaataattataatcgcgtgtcgattgagctggagctgcaatatcgaggcgaattttccgcgacgggtgtgtgtcaagtcgtgcgtccgagggtgcacgcggcaaattgcgcggcgaagttcgtggcgattataattggggcccaaatctgaagtaaggtagattagtgaggtcagctgatcctactattctactgtacttcgaagttgggcccaaataataggcgctaaagccgtcagaatagttatgggtttcgccgcgcaagttgcctcggacgccgcgcgcgggcagtcgattacacaaaccgatcctactgcccatatcccactcattatcaccacatccagtcggcatggtgcaactattgaatccctgatagtgtagactcgggtcagcagaatcttccatccctgcgccgtcgggtacttggtaccaggctcgtctgggtttatgaaagaactgaattgtacatcggaaggccactataagtcattgcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcgcagcttcaatataaataaataaataaataaatgttatgtgtttgtgaataaaactatcatgaactattcaggatgaggtgaatcaggttttgacaaccaacctatggttagagatgcaatggttcac

SEQ ID NO: 62 - DNA, 890 bp, Haemonchus contortus
cgccgcgcgcgaaggagtgtagcagaatgctgcagcaagtaagaaatacagggacaaatacctaacattagaatcatgccaataattataatcgcgtgtcgattgagctggagctgcaatatcgaggcgaattttccgcgacgggtgtgtgtcaagtcgtgcgtccgagggtgcacgcggcaaattgcgcggcgaagttcgtggcgattataattggggcccaaatctgaagtaaggtagattagtgaggtcagctgatcctactattctactgtacttcgaagttgggcccaaataataggcgctaaagccgtcagaatagttatgggtttcgccgcgcaagttgcctcggacgccgcgcgcgggcagtcgattacacaaaccgatcctactgcccatatcccactcattatcaccacatccagtcggcatggtgcaactattgaatccctgatagtgtagactcgggtcagcagaatcttccatccctgcgccgtcgggtacttggtaccaggctcgtctgggtttatgaaagaactgaattgtacatcggaaggccactataagtcattgcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcgcagcttcaatataaataaataaataaataaatgttatgtgtttgtgaataaaactatcatgaactattcaggatgaggtgaatcaggttttgacaaccaacctatggttagagatgcaatggttcgacat

SEQ ID NO: 63 - DNA, 900 bp, Haemonchus contortus
atgctgcagcaggtaagaaatacagggacaaatacctaacactagaatcatgccaataattataatcgcgtgtcgattgagctggagctgcaatatcgaggcgaattttccgcgacgggtgtgtgtcaagtcgtgcgtccgagggtgcacgcggcaaattgcgcggcgaagttcgtggcgattataattggggcccaaatctgaagtaaggtagattagtgaggtcagctgatcctactattctactgtacttcgaagttgggcccaaataataggcgctaaagccgtcagaatagttatgggtttcgccgcgcaagttgcctcggacgcacgacwtgaccgtcccccgtccgcgactgccgcgcgcggtcagtcgattacacaaaccgatcctactgctcatatcccactcattatcacttcatccagtcggcatggtgcaactattgaatccctgatagtatagactcgggtcagcagaatcttctatccctgcgccgtcgggtacttggtaccattctcgtctgggtttatgaaagaactgaattgtacatcggaaggccactatgtataagtcattgcatatgtcatatacatacaccttcaacctcctaacgatttttaatcgaaggggcgtccccaaaacgagatcaatttttgccaagtcttgattcctcactaagacgatccacattgaaaacactttaagaccacttcaggaagttttcttaccgcatgagcactactcattgaactagtactgctgcgcagctttaatatgaataaataaataaataaatgttatgtgtttgtgaataaaactatcatgaactattcaggatgaggtgaatcaggttttgacaaccaacctatggttagagatgcaatggttcgactata

SEQ ID NO: 64 - DNA, 891 bp, Haemonchus contortus
ctgcagcaggtaagaaatacaggggacaaatacctaacactagaatcatgccaataattataatcgcgtgtcgattgagctggagctgcaatatcgaggcgaattttccgcgacgggtgtgtgtcaagtcgtgcgtccgagggtgcacgcggcaaattgcgcggcgaagttcgtggcgattataattggggcccaaatctgaagtaaggtagattagtgaggtcagctgatcctactattctactgtacttcgaagttgggcccaaataataggcgctaaagccgtcagaatagttatgggtttcgccgcgcaagttgcctcggacgcacgacwtgaccgtcccccgtccgcgactgccgcgcgcggtcagtcgattacacaaaccgatcctactgctcatatcccactcattatcacttcatccagtcggcatggtgcaactattgaatccctgatagtatagactcgggtcagcagaatcttctatccctgcgccgtcgggtacttggtaccattctcgtctgggtttatgaaagaactgaattgtacatcggaaggccactatgtataagtcattgcatatgtcatatacatacaccttcaacctcctaacgatttttaatcgaaggggcgtccccaaaacgagatcaatttttgccaagtcttgattcctcactaagacgatccacattgaaaacactttaagaccacttcaggaagttttcttaccgcatgagcactactcattgaactagtactgctgcgcagctttaatatgaataaataaataaataaatgttatgtgtttgtgaataaaactatcatgaactattcaggatgaggtgaatcaggttttgacaaccaacctatggttagagatgcaatggttc

SEQ ID NO: 65 - DNA, 514 bp, Haemonchus contortus
gtcccccgtccgcgactgccgcgcgcggtcagtcgattacacaaaccgatcctactgctcatatcccactcattatcacttcatccagtcggcatggtgcaactattgaatccctgatagtatagactcgggtcagcagaatcttctatccctgcgccgtcgggtacttggtaccattctcgtctgggtttatgaaagaactgaattgtacatcggaaggccactatgtataagtcattgcatatgtcatatacatacaccttcaacctcctaacgatttttaatcgaaggggcgtccccaaaacgagatcaatttttgccaagtcttgattcctcactaagacgatccacattgaaaacactttaagaccacttcaggaagttttcttaccgcatgagcactactcattgaactagtactgctgcgcagctttaatatgaataaataaataaataaatgttatgtgtttgtgaataaaactatcatgaactattcaggatgaggtgaatcagg

SEQ ID NO: 66 - DNA, 885 bp, Haemonchus contortus
cgccgcgcgcgaaggagtatagcagaatgctgcagcaagtaagaaatacagggacaaatacctaacattagaatcatgccaataattataatcgcgtgtcgattgagctggagctgcaatatcgaggcgaattttccgcgacgggtgtgtgtcaagtcgtgcgtccgagggtgcacgcggcaaattgcgcggcgaagttcgtggcgattataattggggcccaaatctgaagtaaggtagattagtgaggtcagctgatcctactattctactgtacttcgaagttgggcccaaataataggcgctaaagccgtcagaatagttatgggtttcgccgcgcaagttgcctcggacgccgcgcgcgggcagtcgattacacaaaccgatcctactgcccatatcccactcattatcaccacatccagtcggcatggtgcaactattgaatccctgatagtgtagactcgggtcagcagaatcttccatccctgcgccgtcgggtacttggtaccaggctcgtctgggtttatgaaagaactgaattgtacatcggaaggccactataagtcattgcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcgcagcttcaatataaataaataaataaataaatgttatgtgtttgtgaataaaactatcatgaactattcaggatgaggtgaatcaggttttgacaaccaacctatggttagagatgcaatggtcg

SEQ ID NO: 67 - DNA, 924 bp, Haemonchus contortus
tgtcgcgtcgcacgaaagagacgccgcgcgcgaaggagtgtagcagaatgctgcagcaagtaagaaatacagggacaaatacctaacattagaatcatgccaataattataatcgcgtgtcgattgagctggagctcgaggcgaattgtccgcgacgggtgtgtgtcaagtcgtgcgtccgagggtgcgcgcggcaaattgcgcggcgaagttcgtgacgattataattggggcccaaatctgaagtaaggtagattagtgaggtcagctgatcctactattctactgtacttcgaagttgggcccaaataataggcgctaaagccgtcagaatagttatgggtttcgccgcgcaagttgcctcggacgcacgacttgaccgtcccccgtccgcgactgccgcgcgcggtcactcgattacacaaaccgatcctactgcccatatcccactcattatcacctcatccagtcggcgtggtgcaaccattgaatccctgatagtatagactcgggtcagcagaatcttccatctctgcgccgtcgggtacttggctcgtctgggtttatgaaagaactgaattgtacatcggaaggccactataagtcattgcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcgcagcttcaatataaataaataaataaatgttatgtgtttgtgaataaaactatcaagaactattcaggatgaggtgaatcaggtttgacaaccaacctatggttagagatgcaatggttcgactata

SEQ ID NO: 68 - DNA, 914 bp, Haemonchus contortus
tgtcgcgtcgcacgaaagagacgccgcgcgcgaaggagtgtagcagaatgctgcagcaagtaagaaatacagggacaaatacctaacattagaatcatgccaataattataatcgcgtgtcgattgagctggagctgcaatatcgaggcgaattgtccgcgacgggtgtgtgtcaagtcgtgcgtccgagggtgcgcgcggcaaattgcgcggcgaagttcgtggcgattgtaattggggcccaaatctgaagtaaggtagattagtgaggtcagctgatcgtactattctactgtacttcgaagttgggcccaaataataggcgctaaagccgtcagaatagttatgggtttcgccgcgcaagttgcctcggacgcacgacttgaccgtcccccgtccgcgactgccgcgcgcggtcagtcgattacacaaaccgatcctactgcccatatcccactcattatcacctcatccagtcggcatggtgcaactattgaatcccttatagtatagactcgggtcagcagaatcttccatccctgcgccgtcgggtacttggtaccaggctcgtctgggtttatgaaagaactgaattgtacattggaaggccactataagtcattrcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcacagcttcaatataaataaataaataaataaatgttatgtgtttgtgaataagactatcatgaactattcaggatgaggtgaatcaggtttgacaaccaaccta

SEQ ID NO: 69 - DNA, 761 bp, Haemonchus contortus
tccgagggtgcgcgcggcaaattgcgcggcgaagttcgtggcgattgtaattggggcccaaatctgaagtaaggtagattagtgaggtcagctgatcgtactattctactgtacttcgaagttgggcccaaataataggcgctaaagccgtcagaatagttatgggtttcgccgcgcaagttgcctcggacgcacgacttgaccgtcccccgtccgcgactgccgcgcgcggtcagtcgattacacaaaccgatcctactgcccatatcccactcattatcacctcatccagtcggcatggtgcaactattgaatcccttatagtatagactcgggtcagcagaatcttccatccctgcgccgtcgggtacttggtaccaggctcgtctgggtttatgaaagaactgaattgtacattggaaggccactataagtcattrcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcacagcttcaatataaataaataaataaataaatgttatgtgtttgtgaataagactatcatgaactattcaggatgaggtgaatcaggttttgacaaccaacctatggtttagagatgcaatggttcgactataa

SEQ ID NO: 70 - DNA, 615 bp, Haemonchus contortus
tgtacttcgaagttgggcccaaataataggcgctaaagccgtcagaatagttatgggtttcgccgcgcaagttgcctcggacgcacgacttgaccgtcccccgtccgcgactgccgcgcgcggtcagtcgattacacaaaccgatcctactgcccatatcccactcattatcacctcatccagtcggcatggtgcaactattgaatcccttatagtatagactcgggtcagcagaatcttccatccctgcgccgtcgggtacttggtaccaggctcgtctgggtttatgaaagaactgaattgtacattggaaggccactataagtcattrcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcacagcttcaatataaataaataaataaataaatgttatgtgtttgtgaataagactatcatgaactattcaggatgaggtgaatcaggtttgacaacc

SEQ ID NO: 71 - DNA, 848 bp, Haemonchus contortus
gctgcagcagtaagaaatacaggggacaaatacctaacattagaatcatgccaataattataatcgcgtgtcgattgagctggagctgcaatatcgaggcgaattgtccgcgacgggtgtgtgtcaagtcgtgcgtccgagggtgcgcgcggcaaattgcgcggcgaagttcgtggcgattgtaattggggcccaaatctgaagtaaggtagattagtgaggtcagctgatcgtactattctactgtacttcgaagttgggcccaaataataggcgctaaagccgtcagaatagttatgggtttcgccgcgcaagttgcctcggacgcacgacttgaccgtcccccgtccgcgactgccgcgcgcggtcagtcgattacacaaaccgatcctactgcccatatcccactcattatcacctcatccagtcggcatggtgcaactattgaatcccttatagtatagactcgggtcagcagaatcttccatccctgcgccgtcgggtacttggtaccaggctcgtctgggtttatgaaagaactgaattgtacattggaaggccactataagtcattgcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcacagcttcaatataaataaataaataaataaatgttatgtgtttgtgaataagactatcatgaactattcaggatgaggtgaatcag

SEQ ID NO: 72 - DNA, 882 bp, Haemonchus contortus
gctgcagcaggtaagaaatacagggacaaatacctaacattagaatcatgccaataattataatcgcgtgtcgattgagctggagctgcaatatcgaggcgaattgtccgcgacgggtgtgtgtcaagtcgtgcgtccgagggtgcgcgcggcaaattgcgcggcgaagttcgtggcgattgtaattggggcccaaatctgaagtaaggtagattagtgaggtcagctgatcgtactattctactgtacttcgaagttgggcccaaatagtaggcgctaaagccgtcagaatagttatgggtttcgccgcgcaagttgcctcggacgcacgacttgaccgtcccccgtccgcgactgccgcgcgcggtcagtcgattacacaaaccgatcctactgcccatatcccactcattatcacctcatccagtcggcatggtgcaactattgaatcccttatagtatagactcgggtcagcagaatcttccatccctgcgccgtcgggtacttggtaccaggctcgtctgggtttatgaaagaactgaattgtacattggaaggccactataagtcattgcatatgtcatatacatacaccttcaacctcctaacgatttttaatcgaaggtgcgtccccaaaacgagatcaatttttgccaagtcttgattcctcactaagacgatcctattgaaaacactttaagaccacttcaggaagttttcttaccgcatgagcactactcattgaactagtactgctgcacagcttcaatataaataaataaataaatgttatgtgtttgtgaataagactatcatgaactattcaggatgaggtgaatcaggttttgacaaccaacctatggttagagatgcaatggttc

SEQ ID NO: 73 - DNA, 874 bp, Haemonchus contortus
atgctgcagcaggtaagaaatacagggacaaatacctaacattagaatcatgccaataattataatcgcgtgtcgattgagctggagctgcaatatcgaggcgaattgtccgcgacgggtgtgtgtcaagtcgtgcgtccgagggtgcgcgcggcaaattgcgcggcgaagttcgtggcgattgtaattggggcccaaatctgaagtaaggtagattagtgaggtcagctgatcgtactattctactgtacttcgaagttgggcccaaataataggcgctaaagccgtcagaatagttatgggtttcgccgcgcaagttgcctcggacgcacgacwtgaccgtcccccgtccgcgactgccgcgcgcggtcagtcgattacacaaaccgatcctactgcccatatcccactcattatcacctcatccagtcggcatggtgcaactattgaatcccttatagtatagactcgggtcagcagaatcttccatccctgcgccgtcgggtacttggtaccaggctcgtctgggtttatgaaagaactgaattgtacattggaaggccactayaagtcattgcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcacagcttcaatataaataaataaataaataaatgttatgtgtttgtgaataagactatcatgaactattcaggatgaggtgaatcaggttttgacaaccaacctatggtta

SEQ ID NO: 74 - DNA, 853 bp, Haemonchus contortus
atgctgcagcaggtaagaaatacaggggacaaatacctaacattagaatcatgccaataattataatcgcgtgtcgattgagctggagctgcaatatcgaggcgaattgtccgcgacgggtgtgtgtcaagtcgtgcgtccgagggtgcgcgcggcaaattgcgcggcgaagttcgtggcgattgtaattggggcccaaatctgaagtaaggtagattagtgaggtcagctgatcgtactattctactgtacttcgaagttgggcccaaataataggcgctaaagccgtcagaatagttatgggtttcgccgcgcaagttgcctcggacgcacgacwtgaccgtcccccgtccgcgactgccgcgcgcggtcagtcgattacacaaaccgatcctactgcccatatcccactcattatcacctcatccagtcggcatggtgcaactattgaatcccttatagtatagactcgggtcagcagaatcttccatccctgcgccgtcgggtacttggtaccaggctcgtctgggtttatgaaagaactgaattgtacattggaaggccactataagtcattgcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcacagcttcaatataaataaataaataaataaatgttatgtgtttgtgaataagactatcatgaactattcaggatgaggtgaatcaggt

SEQ ID NO: 75 - DNA, 891 bp, Haemonchus contortus
gctgcagcagtaagaaatacaggggacaaatacctaacattagaatcatgccaataattataatcgcgtgtcgattgagctggagctgcaatatcgaggcgaattgtccgcgacgggtgtgtgtcaagtcgtgcgtccgagggtgcgcgcggcaaattgcgcggcgaagttcgtggcgattgtaattggggcccaaatctgaagtaaggtagattagtgaggtcagctgatcgtactattctactgtacttcgaagttgggcccaaataataggcgctaaagccgtcagaatagttatgggtttcgccgcgcaagttgcctcggacgcacgacttgaccgtcccccgtccgcgactgccgcgcgcggtcagtcgattacacaaaccgatcctactgcccatatcccactcattatcacctcatccagtcggcatggtgcaactattgaatcccttatagtatagactcgggtcagcagaatcttccatccctgcgccgtcgggtacttggtaccaggctcgtctgggtttatgaaagaactgaattgtacattggaaggccactataagtcattgcatatgtcatatacatacaccttcaacctcctaacgatttttaatcgaaggtgcgtccccaaaacgagatcaatttttgccaagtcttgattcctcactaagacgatccacattgaaaacactttaagaccacttcaggaagttttcttaccgcatgagcactactcattgaactagtactgctgcacagcttcaatataaataaataaataaataaataaatgttatgtgtttgtgaataagactatcatgaactattcaggatgaggtgaatcaggttttgacaaccaacctatggttagagatgcaatggttc

SEQ ID NO: 76 - DNA, 930 bp, Haemonchus contortus
atgtcgcgtcgcacgaaagagacgccgcgcgcgaaggagtgtagcagaatgctgcagcaagtaagaaatacagggacaaatacctaacattagaatcatgccaataattataatcgcgtgtcgattgagctggagctcgaggcgaattgtccgcgacgggtgtgtgtcaagtcgtgcgtccgagggtgcgcgcggcaaattgcgcggcgaagttcgtgacgattataattggggcccaaatctgaagtaaggtagattagtgaggtcagctgatcctactattctactgtacttcgaagttgggcccaaataataggcgctaaagccgtcagaatagttatgggtttcgccgcgcaagttgcctcggacgcacgacttgaccgtcccccgtccgcgactgccgcgcgcggtcactcgattacacaaaccgatcctactgcccatatcccactcattatcacctcatccagtcggcgtggtgcaactattgaatccctgatagtatagactcgggtcagcagaatcttccatctctgcgccgtcgggtacttggctcgtctgggtttatgaaagaactgaattgtacattggaaggccactataagtcattgcatatgtcatatacatacaccttcaacctcctaacgatttttaatcgaaggtgcgtccccaaaacgagatcaatttttgccaagtcttgattcctcactaagacgatcctcattgaaaacactttaagaccacttcaggaagttttcttaccgcatgagcactactcattgaactagtactgctgcacagcttcaatataaataaataaataaataaatgttatgtgtttgtgaataagactatcatgaactattcaggatgaggtgaatcaggttttgacaaccaacctatggttagagatgcaatggttcgactata

SEQ ID NO: 77 - DNA, 882 bp, Haemonchus contortus
gctgcagcagtaagaaatacagggacaaatacctaacattagaatcatgccaataattataatcgcgtgtcgattgagctggagctcgaggcgaattgtccgcgacgggtgtgtgtcaagtcgtgcgtccgagggtgcgcgcggcaaattgcgcggcgaagttcgtgacgattataattggggcccaaatctgaagtaaggtagattagtgaggtcagctgatcctactattctactgtacttcgaagttgggcccaaataataggcgctaaagccgtcagaatagttatgggtttcgccgcgcaagttgcctcggacgcacgacttgaccgtcccccgtccgcgactgccgcgcgcggtcactcgattacacaaaccgatcctactgcccatatcccactcattatcacctcatccagtcggcgtggtgcaactattgaatccctgatagtatagactcgggtcagcagaatcttccatctctgcgccgtcgggtacttggctcgtctgggtttatgaaagaactgaattgtacattggaaggccactataagtcattgcatatgtcatatacatacaccttcaacctcctaacgatttttaatcgaaggtgcgtccccaaaacgagatcaatttttgccaagtcttgattcctcactaagacgatcctcattgaaaacactttaagaccacttcaggaagttttcttaccgcatgagcactactcattgaactagtactgctgcacagcttcaatataaataaataaataaataaatgttatgtgtttgtgaataagactatcatgaactattcaggatgaggtgaatcaggttttgacaaccaacctatggttagagatgcaatggttcgactataatt

SEQ ID NO: 78 - DNA, 896 bp, Haemonchus contortus
tgctgcagcagtaagaaatacagggacaaatacctaacattagaatcatgccaataattataatcgcgtgtcgattgagctggagctgcaatatcgaggcgaattgtccgcgacgggtgtgtgtcaagtcgtgcgtccgagggtgcgcgcggcaaattgcgcggcgaagttcgtggcgattataattggggcccaaatttgaagtaaggcagattagtgaggtcagctgatcctactattctactgtacttcgaagttgggcccaaataataggcgctaaagccgtcagaatagttatgggtttcgccgcgcaagttgcctcggacgcacgacttgaccgtcccccgtccgcgactgccgcgcgcggtcagtcgattacacaaaccgatcctactgcccatatcccactcattatcacctcatccagtcggcatggtgcaactattgaatccctgatagtatagactcgggtcagcagaatcttccatccctgcgccgtcgggtacttggtaccaggctcgtctgggtttatgaaagaactgaattgtacatcggaaggccactataagtcattgcatatgtcatatacatacaccttcaacctcctaacgatttttaatcgaaggtgcgtccccaaaacgagatcaatttttgctaagtcttgattcctcactaagacgatccacattgaaaacactttaagaccacttcaggaagttttcttaccgcatgagcactactcattgaactagtactgctgcgcagcttcaatataaataaataaataaataaataaatgttatgtgtttgtgaataaaactatcatgaactattcaggatgaggtgaatcaggttttgacaaccaacctatggttagagatgcaatggttcgacta

SEQ ID NO: 79 - DNA, 622 bp, Haemonchus contortus
tgaccatccactccaggatttcctgggctaccgctttctccacgaattcctggaaatccgggcatgcctctctcaccttgtggtcctctgaaagtgaactattcagcaagttgcagtaccaatacgaggcatacccttaccttggtcctggtggaccactcggacaaatcgtgcatggtagatcgtcttcaggtgccagctcaacgtcgtagccatcgttaccagctgatccagggttacctggtagcccaggaagaccgtcttcgccggatatcccaggtgcaccaggcagtccctgtgggcatgacaaagggttacagcctgaaattagcccgggaagtcatgagtcaatgagtagtgcatttcttgcctagtaccagtatgaaattcatttcctttgatgattacacaggtgaacaaccttacctatacacccgtctattcgaccctctgaccatggcgctctttttgttcttgaaaaaaatgttggatcattagcattccttcccacattcagaaaaagctgaagctccctccatattctagttgtgtcttcctgaaaaagaccaactcctcaggtcttacttaagcaacgctgattactgtcaaagataaccttgta

SEQ ID NO: 80 - DNA, 619 bp, Haemonchus contortus
ccatccactccaggatttcctgggctaccgctttctccacgaattcctggaaatccgggcatgcctctctcaccttgtggtcctctgaaagtgaactattcagcaagttgcagtaccaatacgaggcatacccttaccttggtcctggtggaccactcggacaaatcgtgcatggtagatcgtcttcaggtgccagctcaacgtcgtagccatcgttaccagctgatccagggttacctggtagcccaggaagaccgtcttcgccggatatcccaggtgcaccaggcagtccctgtgggcatgacaaagggttacagcctgaaattagcccgggaagtcatgagtcaatgagtagtgcatttcttgcctagtaccagtatgaaattcatttcctttgatgattacacaggtgaacaaccttacctatacacccgtctattcgaccctctgaccatggcgctctttttgttcttgaaaaaaatgttggatcattagcattccttcccacattcagaaaaagctgaagctccctccatattctagttgtgtcttcctgaaaaagaccaactcctcaggtcttacttaagcaacgctgattactgtcaaagataaccttgta

SEQ ID NO: 81 - DNA, 622 bp, Haemonchus contortus
gaccatccactccaggatttcctgggctaccgctttctccacgaattcctggaaatccgggcatgcctctctcaccttgtggtcctctgaaagtgaactattcagcaagttgcagtaccaatacgaggcatacccttaccttggtcctggtggaccactcggacaaatcgtgcatggtagatcgtcttcaggtgccagctcaacgtcgtagccatcgttaccagctgatccagggttacctggtagcccaggaagaccgtcttcgccggatatcccaggtgcaccaggcagtccctgtgggcatgacaaagggttacagcctgaaattagcccgggaagtcatgagtcaatgagtagtgcatttcttgcctagtaccagtatgaaattcatttcctttgatgattacacaggtgaacaaccttacctatacacccgtctattcgaccctctgaccatggcgctctttttgttcttgaaaaaaatgttggatcattagcattccttcccacattcagaaaaagctgaagctccctccatattctagttgtgtcttcctgaaaaagaccaactcctcaggtcttacttaagcaacgctgattactgtcaaagataaccttgtac

SEQ ID NO: 82 - DNA, 622 bp, Haemonchus contortus; identical to SEQ ID NO: 79

SEQ ID NO: 83 - DNA, 623 bp, Haemonchus contortus
tgaccatccactccaggatttcctgggctaccgctttctccacgaattcctggaaatccgggcatgcctctctcaccttgtggtcctctgaaagtgaactattcagcaagttgcagtaccaatacgaggcatacccttaccttggtcctggtggaccactcggacaaatcgtgcatggtagatcgtcttcaggtgccagctcaacgtcgtagccatcgttaccagctgatccagggttacctggtagcccaggaagaccgtcttcgccggatatcccaggtgcaccaggcagtccctgtgggcatgacaaagggttacagcctgaaattagcccgggaagtcatgagtcaatgagtagtgcatttcttgcctagtaccagtatgaaattcatttcctttgatgattacacaggtgaacaaccttacctatacacccgtctattcgaccctctgaccatggcgctctttttgttcttgaaaaaaatgttggatcattagcattccttcccacattcagaaaaagctgaagctccctccatattctagttgtgtcttcctgaaaaagaccaactcctcaggtcttacttaagcaacgctgattactgtcaaagataaccttgtac

SEQ ID NO: 84 - DNA, 626 bp, Haemonchus contortus
catgaccatccactccaggatttcctgggctaccgctttctccacgaattcctggaaatccgggcatgcctctctcaccttgtggtcctctgaaagtgaactattcagcaagttgcagtaccaatacgaggcatacccttaccttggtcctggtggaccactcggacaaatcgtgcatggtagatcgtcttcaggtgccagctcaacgtcgtagccatcgttaccagctgatccagggttacctggtagcccaggaagaccgtcttcgccggatatcccaggtgcaccaggcagtccctgtgggcatgacaaagggttacagcctgaaattagcccgggaagtcatgagtcaatgagtagtgcatttcttgcctagtaccagtatgaaattcatttcctttgatgattacacaggtgaacaaccttacctatacacccgtctattcgaccctctgaccatggcgctctttttgttcttgaaaaaaatgttggatcattagcattccttcccacattcagaaaaagctgaagctccctccatattctagttgtgtcttcctgaaaaagaccaactcctcaggtcttacttaagcaacgctgattactgtcaaagataaccttgtact

SEQ ID NO: 85 - DNA, 625 bp, Haemonchus contortus
tgaccatccactccaggatttcctgggctaccgctttctccacgaattcctggaaatccgggcatgcctctctcaccttgtggtcctctgaaagtgaactattcagcaagttgcagtaccaatacgaggcatacccttaccttggtcctggtggaccactcggacaaatcgtgcatggtagatcgtcttcaggtgccagctcaacgtcgtagccatcgttaccagctgatccagggttacctggtagcccaggaagaccgtcttcgccggatatcccaggtgcaccaggcagtccctgtgggcatgacaaagggttacagcctgaaattagcccgggaagtcatgagtcaatgagtagtgcatttcttgcctagtaccagtatgaaattcatttcctttgatgattacacaggtgaacaaccttacctatacacccgtctattcgaccctctgaccatggcgctctttttgttcttgaaaaaaatgttggatcattagcattccttcccacattcagaaaaagctgaagctccctccatattctagttgtgtcttcctgaaaaagaccaactcctcaggtcttacttaagcaacgctgattactggtcaaagataaccttgtact

SEQ ID NO: 86 - DNA, 625 bp, Haemonchus contortus; identical to SEQ ID NO: 85

SEQ ID NO: 87 - DNA, 620 bp, Haemonchus contortus
ccatccactccaggatttcctgggctaccgctttctccacgagttcctggaaatccgggcatgcctctttcaccttgtggtcccctgaaagtgaactaattcattaagttgcagtacaaagacaaggcatacccttaccttggtcctggtggaccactcggacaaattgtgcatggtagatcgtcttcaggtgctaactctacgtcgtagccatcgttgccagctgatccagggttacctggtagcccaggaagaccgtcttcgccggatattccaggtgcaccaggcagtccttgtgggcaggacaaagggttacagcctggaattagcccggcaagtcatgaatcaatgagtattgcatttcttgcctagtaccagcatgaaattcatttcctttgatgattacacaggtgaacaaccttacctatacacccgtctagccggccctctgaccatggtgccctttttgttcttgaaaaatatgttggatcattactattccttcccacattcaggaaaagctgaagctccctccatattcgagttgtgtcctcctgaaaaagaccaactcctcaagtctttccgaagcaacgctgattactgtcaaagatgaccttgta

SEQ ID NO: 88 - DNA, 508 bp, Haemonchus contortus
ggrakccgggcatgcctctctcaccttgtggtcctctgaaagtgaamtaytyaacaagttgcagtaccaagacraggcatacccttaccttggtcctggtggaccacttgggcaaatygtgcatggtagatcgtcttcaggtgctagctccacgtcgtagccatcgttgccagctgatccagggttacctggtagcccaggaagaccgtcttcgccggatattccaggtgcaccaggcagtccytgtgggcacgacaaagggttacagcctgaaattagccsrgcaaatcrtgaatcawtgagtartacatttcttgccyagtaccagtatgacattcattycctttratgattacayaggtgaacaaccttacctatacacccgtctattcgaccctctgaccakggtgccctttttgttcttgaaaaatatrtgggatcatyactattycttcccacattcagaaaaagctgragctccctccatattcgagttgtgtcttcctga

SEQ ID NO: 89 - DNA, 508 bp, Haemonchus contortus; identical to SEQ ID NO: 88

SEQ ID NO: 90 - DNA, 686 bp, Haemonchus contortus
gaccatccactccaggatttcctgggctaccgctttctccacgaattcctggaaatccgggcatgcctctctcaccttgtggtcctctgaaaaagaactacttaacaagttgcagtaccaagacaaagcatacccttaccttggtcctggtggaccacttgggcaaattgtgcatggtagatcgtcttcaggtgctagctccacgtcgtagccatcgttgccagctgatccagggttacctggtagcccaggaagaccgtcttcgccggatattccaggtgcaccaggcagtccctgtgggcacgacaaagggttacagcctgaaattagcccggcaaatcatgaatcattgagtagtacatttcttgcccagtaccagtatgaaattcatttcctttaatgattacacaggtgaacaaccttacctatacacccgtctattcgaccctctgaccatggtgccctttttgttcttgaaaaatatgttggatcattactattccttcccacattcaggaaaagctgaagctccctccatattcgagttgtgtcttcctgaagaagggtaactctccaggttcttcttgagcaacgctgtttactgttttgacttgatgttttgttaactgctgttatatcgccgcagtacgcgtaaggctgattattgtcaaagataaccttgtact

SEQ ID NO: 91 - DNA, 688 bp, Haemonchus contortus
catgaccatccactccaggatttcctgggctaccgctttctccacgaattcctggaaatccgggcatgcctctctcaccttgtggtcctctgaaaaagaactacttaacaagttgcagtaccaagacaaagcatacccttaccttggtcctggtggaccacttgggcaaattgtgcatggtagatcgtcttcaggtgctagctccacgtcgtagccatcgttgccagctgatccagggttacctggtagcccaggaagaccgtcttcgccggatattccaggtgcaccaggcagtccctgtgggcacgacaaagggttacagcctgaaattagcccggcaaatcatgaatcattgagtagtacatttcttgcccagtaccagtatgaaattcatttcctttaatgattacacaggtgaacaaccttacctatacacccgtctattcgaccctctgaccatggtgccctttttgttcttgaaaaatatgttggatcattactattccttcccacattcaggaaaagctgaagctccctccatattcgagttgtgtcttcctgaagaagggtaactctccaggttcttcttgagcaacgctgtttactgttttgacttgatgttttgttaactgctgttatatcgccgcagtacgcgtaaggctgattactgtcaaaaataaccttgtac

SEQ ID NO: 92 - DNA, 683 bp, Haemonchus contortus
gccatccactccaggatttcctgggctaccgctttctccacgaattcctggaaatccgggcatgcctctctcaccttgtggtcctctgaaaaagaactacttaacaagttgcagtaccaagacaaagcatacccttaccttggtcctggtggaccacttgggcaaattgtgcatggtagatcgtcttcaggtgctagctccacgtcgtagccatcgttgccagctgatccagggttacctggtagcccaggaagaccgtcttcgccggatattccaggtgcaccaggcagtccctgtgggcacgacaaagggttacagcctgaaattagcccggcaaatcatgaatcattgagtagtacatttcttgcccagtaccagtatgaaattcatttcctttaatgattacacaggtgaacaaccttacctatacacccgtctattcgaccctctgaccatggtgccctttttgttcttgaaaaatatgttggatcattactattccttcccacattcaggaaaagctgaagctccctccatattcgagttgtgtcttcctgaagaagggtaactctccaggttcttcttgagcaacgctgtttactgttttgacttgatgttttgttaactgctgttatatcgccgcagtacgcgtaaggctgattaytgtcaaarataaccttgta

SEQ ID NO: 93 - DNA, 645 bp, Haemonchus contortus
tccatacccattgaccatccactccaggattccctgggctaccgctttctccacggattcctggaaatccgggcatgcctctctcaccttgtggtcctctgaaacagaactacttaacaagttgcagtaccaagacaaagcatacccttaccttggtcctggtggaccacttgggcaaattgtgcatggtagatcgtcttcaggtgccagctccacgtcgtagccatcgttgccagctgatccagggttacctggtagccctgggagcccgtcttcgccggatataccaggtgcaccaggcagtccctgtgggcacgacaaagggttacagcctgaaattagcccggcaaatcatgaatcaatgagtagtacatttcttgcctagtaccagtatgaaattcatttcctttaatgattacacaggtgaataaccttacctatacacccgtctattcgaccctctgaccagggtgccctttttgttcttgaaaaaaatgttggatcatcactgttccttcccacattcaggaaaagctgaagttctctccatattcgagtcgtgtcttcctgaaacggccaactcctcaggtctttcacaagcaacgctgattactgtcaaagataaccttgtacttcttcgccttt

SEQ ID NO: 94 - DNA, 644 bp, Haemonchus contortus
tccatacccattgaccatccactccaggattccctgggctaccgctttctccacggattcctggaaatccgggcatgcctctctcaccttgtggtcctctgaaacagaactacttaacaagttgcagtaccaagacaaagcatacccttaccttggtcctggtggaccacttgggcaaattgtgcatggtagatcgtcttcaggtgccagctccacgtcgtagccatcgttgccagctgatccagggttacctggtagccctgggagcccgtcttcgccggatataccaggtgcaccaggcagtccctgtgggcacgacaaagggttacagcctgaaattagcccggcaaatcatgaatcaatgagtagtacatttcttgcctagtaccagtatgaaattcatttcctttaatgattacacaggtgaataaccttacctatacacccgtctattcgaccctctgaccagggtgccctttttgttcttgaaaaaaatgttggatcatcactgttccttcccacattcaggaaaagctgaagttctctccatattcgagtcgtgtcttcctgaaacggccaactcctcaggtctttcacaagcaacgctgattactgtcaaagataaccttgtacttcttcgcctt

SEQ ID NO: 95 - DNA, 645 bp, Haemonchus contortus
tccatacccattgaccatccactccaggattccctgggctaccgctttctccacggattcctggaaatccgggcatgcctctctcaccttgtggtcctctgaaacagaactacttaacaagttgcagtaccaagacaaagcatacccttaccttggtcctggtggaccacttgggcaaattgtgcatggtagatcgtcttcaggtgccagctccacgtcgtagccatcgttgccagctgatccagggttacctggtagccctgggagcccgtcttcgccggatataccaggtgcaccaggcagtccctgtgggcacgacaaagggttacagcctgaaattagcccggcaaatcatgaatcaatgagtagtacatttcttgcctagtaccagtatgaaattcatttcctttaatgattacacaggtgaataaccttacctatacacccgtctattcgaccctctgaccagggtgccctttttgttcttgaaaaaaatgttggatcatcactgttccttcccacattcaggaaaagctgaagttctctccatattcgagtcgtgtcttcctgaaacggccaactcctcaggtctttcacaagcaacgctgattactgtcaaagataaccttgtacttctttcgcctt

SEQ ID NO: 96 - DNA, 650 bp, Haemonchus contortus
ttgaccatccactccaggatttcctgggctaccgctttctccacgaattcctggaaatccgggcatgcctctctcaccttgtggtcctctgaaaaagaactacttaacaagttgcagtaccaagacaaagcatatccttaccttggtcctggtggaccacttgggcaaattgtgcatggtagatcgtcttcaggtgctagctccacgtcgtagccatcgttgccagctgatccagggttacctggtagcccaggaagaccgtcttcrccggatattccaggtgcaccaggcaktccctgtgggcacgacaaagggttrcagcctggaattagccttgcaaatcatgaaccaatraatagtgcatttcttgcctagtaccagtatgaaattcatttcctttgatgattacacaggtgaacaaccwtacctatacacccatctattcgaccctctgaccatggtgccctttttgttcttgaaaaatatgttggatcattactattccttcccacattcagaaaaagctgaagctccctccatattcgagttgtgtcttcctggaaaaaactgactcttcaggtctttctcargcaacgctgattaytgtcaaagataaccttgtacttctttgccttcaattcaatgttgga

SEQ ID NO: 97 - DNA, 605 bp, Haemonchus contortus
tggaaatccgggcatgcctctctcaccttgtggtcctctgaaaaagaactacttaacaagttgcagtaccaagasaaagcatatccttaccttggtcctggtggaccacttgggcaaattgtgcatggtagatcgtcttcaggtgctagctccacgtcgtakccatsgttgccagctgatccagggttacctggtagcccargaagaccgtcttcrccggatattccaggtgcaccaggcaktccctgtgggcacgacaaagggttrcagcctggaattakccttgcaaatcatgaaccaataaatagtgcatttcttgcctagtaccagtatgaaattcatttcctttgatgattacacaggtgaacaaccatacctatacacccatctattcgaccctctgaccatggtgccctttttgttcttgaaaaatatgttggatcattactattccttcccacattcagaaaaagctgaagctccctccatattcgagttgtgtcttcctggaaaaaactgactcttcaggtctttctcargcaacgctgattaytgtcaaagataaccttgtacttctttgccttcaattcaatgttggatacttc

SEQ ID NO: 98 - DNA, 682 bp, Haemonchus contortus
ccatccactccgggatttcctgggctaccgctttctccacgaattcctggaaatccgggcatgcctctctcaccttgtggtcctctgaaaaagaactacttaacaagttgcagtaccaagacaaagcatacccttaccttggtcctggtggaccacttgggcaaattgtgcatggtagatcgtcttcaggtgctagctccacgtcgtagccatcgttgccagctgatccagggttacctggtagcccaggaagaccgtcttcgccggatattccaggtgcaccaggcagtccctgtgggcacgacaaagggttacagcctgaaattagtccgacaagtcatgaatcaatgagtagtgcatttcttgcctagtaccagtatgaaattcatttcctttgatgattacacaggtgaacaaccatacctatacacccgtctattcgaccctctgaccatggtgccctttttgttcttgaaaaatatgttggatcgttactattccttcccacattcaggaaaagctgaagctccctccagattcgagttgtgtcttcctgaaaaagggtaactcttcagcttctccttgagcaacgctgattactgtttggacttgatgttttgttaactgctgttatatcgccgcagtacgcgtaaggctgattactgtcgaaaataaccttgta

SEQ ID NO: 99 - DNA, 643 bp, Haemonchus contortus
ttccataacccattgaccatccactccaggatttcctgggctaccgctttctccacggattcctggaaatccgggcatgcctctttcaccttgtggtcccctgaaagtgaactattcaacaagttgcagtacgaagggatataagggatacatctaccttggtcctggtggaccacttgggcaaattgtacatggtagatcgtcttcaggtgccagctccacgtcgtagccatcgttgccagctgatccagggttacctggtagcccaggaagaccgtcttcgccggatattccaggtgcaccaggcagtccttgtgggcacgacaaagggttgcagcctgaaattagccaggcaaatcatgaatcaatgagtagtacatttcttgcccactaccagtatgaaatccatttgcttagatgattacacaggtgaacaaccttacctatacacccgtctaatcgaccctctgaccagggtgccctttttgttcttgagaaatatgttggatcattactattccttcccacattcagaaaaagctgaagctccctccatattcgagttgtgtcttcctgaaaaggactgacctttcaggtctttcttaagcaacgctgattactgccaaaggtaaccttgtacttc

SEQ ID NO: 100 - DNA, 644 bp, Haemonchus contortus
tttccataacccattgaccatccactccaggatttcctgggctaccgctttctccacggattcctggaaatccgggcatgcctctttcaccttgtggtcccctgaaagtgaactattcaacaagttgcagtacgaagggatataagggatacatctaccttggtcctggtggaccacttgggcaaattgtacatggtagatcgtcttcaggtgccagctccacgtcgtagccatcgttgccagctgatccagggttacctggtagcccaggaagaccgtcttcgccrgatattccaggtgcaccaggcagtccttgtgggcacgacaaagggttgcagcctgaaattagccaggcaaatcatgaatcaatgagtagtacatttcttgcccastaccagtatgraatccatttgcttwgatgattacacaggtgaacaaccttacctatacacccgtctawtcgaccctctgaccakggtgccctttttgttcttgaraaatatgttggatcattactattcctkccmacattcagaaaaagctgaagctccctccatattcgagtwgtgtcttcctgaaaaggactgacctttcaggtctttcttaagcaacgctgattactgccaaagrtaaccttgtacttc

SEQ ID NO: 101 - DNA, 684 bp, Haemonchus contortus
gaccatccactccgggatttcctgggctaccgctttctccacgaattcctggaaatccgggcatgcctctctcaccttgtggtcctctgaaaaagaactacttaacaagttgcagtaccaagacaaagcatacccttaccttggtcctggtggaccacttgggcaaattgtgcatggtagatcgtcttcaggtgctagctccacgtcgtagccatcgttgccagctgatccagggttacctggtagcccaggaagaccgtcttcgccggatattccaggtgcaccaggcagtccctgtgggcacgacaaagggttacagcctgaaattagtccgacaagtcatgaatcaatgagtagtgcatttcttgcctagtaccagtatgaaattcatttcctttgatgattacacaggtgaacaaccatacctatacacccgtctattcgaccctctgaccatggtgccctttttgttcttgaaaaatatgttggatcgttactattccttcccacattcaggaaaagctgaagctccctccagattcgagttgtgtcttcctgaaaaagggtaactcttcagcttctccttgagcaacgctgattactgtttggacttgatgttttgttaactgctgttatatcgccgcagtacgcgtaaggctgattactgtcgaaaataaccttgta

SEQ ID NO: 102 - DNA, 625 bp, Haemonchus contortus
ttgaccatccactccaggatttcctgggctaccgctttctccacggattcctggaaatccgggcatgcctctttcaccttgtggtcccctgaaagtgaactattcaacaagttgcagtacgaagacaagggatacatctaccttggtcctggtggaccacttgggcaaattgtacatggtagatcgtcttcaggtgccagctccacgtcgtagccatcgttgccagctgatccagggttacctggtagcccaggaagaccgtcttcgccagatattccaggtgcaccgggcagtccttgtgggcacgacaaagggttgcagcctgaaattagccaggcaaatcatgaatcaatgagtagtacatttcttgcccagtaccagtatgaaatccatttgctttgatgattacacaggtgaacaaccttacctatacacccgtctaatcgaccctctgaccagggtgccctttttgttcttgagaaatatgttggatcattactattccttcccacattcagaaaaagctgaagctccctccatattcgagttgtgtcttcctgaaaaggactgacctttcaggtctttcttaagcaacgctgattactgccaaagataaccttgtact

SEQ ID NO: 103 - DNA, 684 bp, Haemonchus contortus
gaccatccactccaggatttcctgggctaccgctttctccacgaattcctggaaatccgggcatgcctctctcaccttgtggtcctctgaaaaagaactacttaacaagttgcagtaccaagacaaagcatacccttaccttggtcctggtggaccacttgggcaaattgtgcatggtagatcgtcttcaggtgctagctccacgtcgtagccatcgttgccagctgatccagggttacctggtagcccaggaagaccgtcttcgccggatattccaggtgcaccaggcagtccctgtgggcacgacaaagggttacagcctgaaattagcccggcaaatcatgaatcattgagtagtacatttcttgcccagtaccagtatgaaattcatttcctttaatgattacacaggtgaacaaccttacctatacacccgtctattcgaccctctgaccatggtgccctttttgttcttgaaaaatatgttggatcattactattccttcccacattcaggaaaagctgaagctccctccatattcgagttgtgtcttcctgaagaagggtaactctccaggttcttcttgagcaacgctgtttactgttttgacttgatgttttgttaactgctgttatatcgccgcagtacgcgtaaggctgattactgtcaaaaataaccttgta

SEQ ID NO: 104 - DNA, 628 bp, Haemonchus contortus
tgaccatccactccaggatttcctgggctaccgctttctccacgaattcctggaaatccgggcatgcctctttcaccttgtggtcccctgaaagtgaactattcaacaagttgcagtacgaagggatataagggatacatctaccttggtcctggtggaccacttgggcaaattgtacatggtagatcgtcttcaggtgccagctccacgtcgtagccatcgttgccagctgatccagggttacctggtagcccaggaagaccgtcttcgccggatattccaggtgcaccaggcagtccttgtgggcacgacaaagggttgcagcctgaaattagccaggcaaatcatgaatcaatgagtagtacatttcttgcccagtaccagtatgaaatccatttgcttagatgattacacaggtgaacaaccttacctatacacccgtctaatcgaccctctgaccagggtgccctttttgttcttgagaaatatgttggatcattactattccttcccacattcagaaaaagctgaagctccctccatattcgagttgtgtcttcctgaaaaggactgacctttcaggtctttcttaagcaacgctgattactgccaaaggtaaccttgtact

SEQ ID NO: 105 - DNA, 685 bp, Haemonchus contortus
ccatccactccaggatttcctgggctaccgctttctccacgaattcctggaaatccgggcatgcctctctcaccttgtggtcctctgaaaaagaactacttaacaagttgcagtaccaagacaaagcatacccttaccttggtcctggtggaccacttgggcaaattgtgcatggtagatcgtcttcaggtgctagctccacgtcgtagccatcgttgccagctgatccagggttacctggtagcccaggaagaccgtcttcgccggatattccaggtgcaccaggcagtccctgtgggcacgacaaagggttacagcctgaaattagcccggcaaatcatgaatcattgagtagtacatttcttgcccagtaccagtatgaaattcatttcctttaatgattacacaggtgaacaaccttacctatacacccgtctattcgaccctctgaccatggtgccctttttgttcttgaaaaatatgttggatcattactattccttcccacattcaggaaaagctgaagctccctccatattcgagttgtgtcttcctgaagaagggtaactctccaggttcttcttgagcaacgctgtttactgttttgacttgatgttttgttaactgctgttatatcgccgcagtacgcgtaaggctgattactgtcaaaaataaccttgtactc

SEQ ID NO: 106 - DNA, 685 bp, Haemonchus contortus
gaccatccactccaggatttcctgggctaccgctttctccacgaattcctggaaatccgggcatgcctctctcaccttgtggtcctctgaaaaagaactacttaacaagttgcagtaccaagacaaagcatacccttaccttggtcctggtggaccacttgggcaaattgtgcatggtagatcgtcttcaggtgctagctccacgtcgtagccatcgttgccagctgatccagggttacctggtagcccaggaagaccgtcttcgccggatattccaggtgcaccaggcagtccctgtgggcacgacaaagggttacagcctgaaattagcccggcaaatcatgaatcattgagtagtacatttcttgcccagtaccagtatgaaattcatttcctttaatgattacacaggtgaacaaccttacctatacacccgtctattcgaccctctgaccatggtgccctttttgttcttgaaaaatatgttggatcattactattccttcccacattcaggaaaagctgaagctccctccatattcgagttgtgtcttcctgaagaagggtaactctccaggttcttcttgagcaacgctgtttactgttttgacttgatgttttgttaactgctgttatatcgccgcagtacgcgtaaggctgattactgtcaaaaataaccttgtat

SEQ ID NO: 107 - DNA, 686 bp, Haemonchus contortus
gaccatccactccaggatttcctgggctaccgctttctccacgaattcctggaaatccgggcatgcctctctcaccttgtggtcctctgaaaaagaactacttaacaagttgcagtaccaagacaaagcatacccttaccttggtcctggtggaccacttgggcaaattgtgcatggtagatcgtcttcaggtgctagctccacgtcgtagccatcgttgccagctgatccagggttacctggtagcccaggaagaccgtcttcgccggatattccaggtgcaccaggcagtccctgtgggcacgacaaagggttacagcctgaaattagcccggcaaatcatgaatcattgagtagtacatttcttgcccagtaccagtatgaaattcatttcctttaatgattacacaggtgaacaaccttacctatacacccgtctattcgaccctctgaccatggtgccctttttgttcttgaaaaatatgttggatcattactattccttcccacattcaggaaaagctgaagctccctccatattcgagttgtgtcttcctgaagaagggtaactctccaggttcttcttgagcaacgctgtttactgttttgacttgatgttttgttaactgctgttatatcgccgcagtacgcgtaaggctgattactgtcaaaaataaccttgtact

SEQ ID NO: 108 - DNA, 640 bp, Haemonchus contortus
tttccatacccattgaccatccactccaggatttcctgggctaccgctttctccacgaattcctggaaatccgggcatgcctctttcaccttgtggtcctctgaaaaaagaactattcaacaagttgcagttccaagaagaggcatatcctcaccttggtcctggtggaccacttgggcaaattgtgcatggtagatcgtcttcaggtgccagctccacgtcgtagccatcgttgccagctgatccagggttacctggtagcccaggaagaccgtcttcgccggatattccaggtgcaccaggcagtccctgtgggcacgacaaagggttacagcctgaaattagcccagcaaatcatgaatcaatgagtagtatatttcttgcctagtaccagtatgaaattcattttctttgatgattacacagcttacctatacacccgtctattcgaccctctgaccatggtgccctttttgttcttgaaaaaaacgttggatcattactattccttcccacattcaggaagagctgaagctctctccatattcgagtcgtgtcttcctgaagaagactgactctcaagtcttttttgagcaattagctgattactgtcaaaaccaaccttgtacttcttcgcctt

SEQ ID NO: 109 - DNA, 655 bp, Haemonchus contortus
tttccatacccattgaccatccactccaggatttcctgggctaccgctttctccacgaattcctgggaagccgggcatgcctctctcaccttgtggtcctctgaaagtgaaatattcaacaagttgcagtaccaagacgaggcatacccttaccttggtcctggtggaccacttgggcaaatcgtgcatggtagatcgtcttcaggtgctagctccacgtcgtagccatcgttgccagctgatccagggttacctggtagcccaggaagaccgtcttcgccggatattccaggtgcaccaggcagtccttgtgggcacgacaaagggttacagcctgaaattagccgagcaaatcgtgaatcaatgagtaatacatttcttgcctagtaccagtatgacattcattccctttgatgattacataggtgaacaaccttacctatacacccgtctattcgaccctctgaccagggtgccctttttgttcttgaaaaaaatatgggatcatcactatttcttcccacattcagaaaaagctggagctccctccatattcgagttgtgtcttcctgaaaaagaccaactcctaaggcctttcacaagcaacgctgattactgtcaaagataaccttgtacttctttcgcctttaaaaga

SEQ ID NO: 110 - DNA, 709 bp, Haemonchus contortus
tccatacccattgaccatctactccaggatttcctgggctaccgctttctccacgaattcctggaaatccgggcatgcctctctcaccttgtggtcctctgaaaaagaactacttaacaagttgcagtaccaagacaaagcatacccttaccttggtcctggtggaccacttgggcaaattgtgcatggtagatcgtcttcaggtgccagctccacgtcgtagccatcgttgccagctgatccagggttacctggtagcccaggaagaccgtcttcgccggatattccaggtgcaccaggcagtccctgtgggcacgacaaagggttacagcctgaaattagccttgcaaatagtgaaccaataaatagttcatttcttgcatagtactagtatgcaatttatttcctttgattagtacacaaggtgaatgaccttacctatacacccgtcaagccgaccttctgaccagggtgccctttttgttcttgaaaaaaatgttggatcattactattccttcccacattcaggaaaagctgaagctccctccatattcgggttgtgtcttcctgaagaaaggtaactcttcaggtccttcttgagcaacgctgattactgttttgacttgatgttttgttaattgctgttatatcgccgcagtacgcgtaaggctgattactgtcaaaaataaccttgtacttctttcgcct

SEQ ID NO: 111 - DNA, 648 bp, Haemonchus contortus
tttccatacccattgaccatccactccaggatttcctgggctaccgctttctccacgaattcctgggaagccgggcatgcctctctcaccttgtggtcctctgaaagtgaaatattcaacaagttgcagtaccaagacgaggcatacccttaccttggtcctggtggaccacttgggcaaatcgtgcatggtagatcgtcttcaggtgctagctccacgtcgtagccatcgttgccagctgatccagggttacctggtagcccaggaagaccgtcttcgccggatattccaggtgcaccaggcagtccttgtgggcacgacaaagggttacagcctgaaattagccgagcaaatcgtgaatcaatgagtaatacatttcttgcctagtaccagtatgacattcattccctttgatgattacataggtgaacaaccttacctatacacccgtctattcgaccctctgaccagggtgccctttttgttcttgaaaaaaatatgggatcatcactatttcttcccacattcagaaaaagctggagctccctccatattcgagttgtgtcttcctgaaaaagaccaactcctaaggcctttcacaagcaacgctgattactgtcaaagataaccttgtacttctttcgcctt

SEQ ID NO: 112 - protein, 13 aa, Artificial Sequence
  Deduced amino acid sequence of frame 2
LLLYRRSTRKADY

SEQ ID NO: 113 - protein, 6 aa, Artificial Sequence
  Deduced amino acid sequence of reverse frame 1
QNIKSK

SEQ ID NO: 114 - protein, 9 aa, Artificial Sequence
  Deduced amino acid sequence of reverse frame 3
QQLTKHQVK

SEQ ID NO: 115 - DNA, 63 bp, Haemonchus contortus
ttttgacttgatgttttgttaactgctgttatatcgccgcagtacgcgtaaggctgattactg

SEQ ID NO: 116 - protein, 4 aa, Artificial Sequence
  typical nAChR subunit motif
YXCC

(19) United States
(12) Patent Application Publication
(10) Pub. No.: US 2016/0289773 A1
(43) Pub. Date: Oct. 6, 2016

(54) DETECTION AND MONITORING OF RESISTANCE TO AN IMIDAZOTHIAZOLE ANTI-HELMINTHIC IN NEMATODES

(71) Applicant: THE ROYAL INSTITUTION FOR THE ADVANCEMENT OF LEARNING / MCGILL UNIVERSITY, Montréal (CA)

(72) Inventors: Roger Prichard, Saint-Anne-de-Bellevue (CA); Virginie Barrere, Ottawa (CA); Robin Beech, Longueuil (CA); Cedric Neveu, Nouzilly (FR)

(73) Assignee: THE ROYAL INSTITUTION FOR THE ADVANCEMENT OF LEARNING / MC GILL UNIVERSITY, Montréal, QC (CA)

(21) Appl. No.: 14/903,671

(22) PCT Filed: Jul. 11, 2014
(86) PCT No.: PCT/CA2014/050661

§ 371 Date: Jan. 8, 2016

Related U.S. Application Data
(60) Provisional application No. 61/845,237, filed on Jul. 11, 2013.

(51) Int. Cl. C12Q 1/68

(52) U.S. Cl. CPC C12Q 1/6888; C12Q 2600/106; C12Q 2600/156

(57) ABSTRACT

The present disclosure concerns the determination of the resistance or the susceptibility of a nematode to an imidazothiazole anti-helmintic based on the assessment of the presence of an indel in the Hco-acr-8 gene (or the Hco-acr-8 gene ortholog). The present disclosure also provides tools and commercial packages for making such assessment.

### CROSS-REFERENCE TO RELATED APPLICATIONS AND DOCUMENTS

This application claims priority from U.S. provisional patent application 61/845,237 filed on Jul. 11, 2013 and herewith incorporated in its entirety. A sequence listing in electronic format is also filed with the present application and its content is incorporated in its entirety.

### TECHNOLOGICAL FIELD

The present disclosure relates to the detection, and optionally the monitoring, of resistance to imidazothiazole derivatives, such as levamisole and tetramisole, in nematodes based on the characterization of the genomic DNA of the Hco-acr-8 gene (or the Hco-acr-8 gene ortholog).

### BACKGROUND

The Hco-acr-8 gene encodes a ligand-gated ion-channel α-type subunit that is a component of a levamisole sensitive acetylcholine receptor (L-AChR1) in Haemonchus contortus. Hco-acr-8 is related to Lev-8, in the free living nematode, Caenorhabditis elegans. The loss of Lev-8 and its replacement by Hco-acr-8 is supported by experimental evidence in C. elegans with expression in body muscles and a few head and tail neurons. The Hco-ACR-8 sequence contains typical nAChR subunit motifs including YXCC in loop C and the 15 amino acid cys-loop. Trychostrongylid ACR-8 has 68-69% homology with C. elegans ACR-8 and shares common conserved amino acids sequences with Cel-LEV-8, making Hco-ACR-8 the closest homologue of Cel-LEV-8. The Hco-acr-8 gene contains 15 exons and spans more than 19 kb of gDNA, producing a full transcript of 2101 bp.

The H. contortus transcriptome was analyzed in levamisole-resistant and levamisole-susceptible isolates and the expression of a truncated transcript of Hco-acr-8 mRNA (Hco-acr-8b) was found in three levamisole-resistant isolates (Kokstad, Cedara and RHS6). The same truncated transcript was also found in a multi-resistant isolate of H. contortus UGA/2004 and two isolates from Australia LevR and Wallangra 2003. The isoform Hco-acr-8b mRNA was sequenced (acquisition number: GU168769) and it was observed that the sequence contained the 2 first exons and a part of intron 2 of Hco-acr-8 (Acquisition number: EU006785). The sequence between exon 2 and exon 3, included in the truncated transcript is referred to as exon 3b. In the full length transcript, exon 3b is spliced out, but is retained in the truncated mRNA. In addition, it was also reported that the expression level of Hco-acr-8b was detected only in adult stages and not free-living stages.

A levamisole sensitive ACh receptor from H. contortus is composed of subunits Hco-ACR-8, Hco-UNC-63, Hco-UNC-38 and Hco-UNC-29. A functional receptor that lacks Hco-ACR-8 can be produced and is significantly less sensitive to levamisole. It is possible that ACh receptors lacking Hco-ACR-8 are expressed in worms producing the truncated Hco-ACR-8b and this could be a possible mechanism of resistance to levamisole. The use of RNA as a diagnostic tool for detecting resistance however poses technical problems.

It would be highly desirable to be provided with a method and tools for detecting/monitoring levamisole resistance in nematodes which do not rely on the assessment of presence of the truncated Hco-acr-8 transcript.

### BRIEF SUMMARY

One aim of the present disclosure is to provide methods and tools for determining the susceptibility/resistance of a nematodes towards imidazothiazole anti-helminthics based on the characterization of the Hco-acr-8 (or the Hco-acr-8 gene ortholog) genomic sequence. As it will be further discusses herein, the determination of the presence or absence of an indel in the Hco-acr-8 gene (or its corresponding gene ortholog) is indicative of the susceptibility or resistance of a nematode to imidazothiazole anti-helminthics. Assessment of resistance of imidazothiazole anti-helminthics by using a genomic marker is advantageous because it does not rely on the cumbersome isolation and amplification of RNA from the nematodes or on the interpretation of the level of transcript or protein expression. Analyses conducted on genomic DNA (gDNA) is, in general easier, more robust and faster than analyses conducted on mRNA or protein. Characterization of mRNA of nematode egg has been shown to be difficult or unreliable. Further, the methods described herein can successfully applied to adult nematode as well as nematodes at the larval stage (L1, L2, L3 or L4 stages) and even a nematode egg.

In a first aspect, the present disclosure provides a method for assessing the susceptibility of a nematode to an imidazothiazole anti-helminthic. Broadly, the method comprises providing a genomic DNA sample of the nematode comprising a Hco-acr-8 gene or a Hco-acr-8 gene ortholog as well as determining the presence or absence of an indel having the sequence of SEQ ID NO: 9 and being located in the Hco-acr-8 gene or the Hco-acr-8 gene ortholog. In an embodiment, the nucleic acid residue at position 4 of SEQ ID NO: 9 is T and/or the nucleic acid residue at position 61 of SEQ ID NO: 9 is C. The method also provides characterizing the nematode as susceptible to the imidazothiazole anti-helminthic when the indel sequence is determined to be present in the genomic DNA sample and as resistant to the imidazothiazole anti-helminthic when the indel sequence is determined to be absent from the genomic DNA sample. In an embodiment, the imidazothiazole anti-helminthic is levamisole. In another embodiment, the nematode is from a Trichostrongylidae family (from a Haemonchus genus for example, such as Haemonchus contortus). In an embodiment, the genomic DNA is from a nematode egg. In an embodiment, prior to the determination step, a nucleic-acid synthetic copy of the genomic DNA comprising the Hco-acr-8 gene or the Hco-acr-8 gene ortholog is made and used to determine the presence or absence of the indel. In an embodiment, the indel is located between exon 2 and exon 3 of the Hco-acr-8 gene or the Hco-acr-8 gene ortholog. In an embodiment, the method can further comprise, in the determination step, (i) contacting the genomic DNA with at least one pair of primers specific for the vicinity of the indel located in the Hco-acr-8 gene or the Hco-acr-8 gene ortholog under conditions to form a complex between the genomic DNA and the pair of primers, (ii) amplifying the genomic DNA with the at least one pair of primers to provide at least one amplicon, and (iii) determining the presence of the indel in the at least one amplicon; and/or, in the characterization step, characterizing the nematode as susceptible to the imidazothiazole anti-helminthic when the indel is determined to be present in the at least one amplicon and as resistant to the imidazothiazole anti-helminthic when the indel is determined to be absent from the at least one amplicon. In still another embodiment, the at least one pair of primers comprises a first primer and a second primer, wherein the first primer has a nucleic acid sequence corresponding to a first location in the Hco-acr-8g gene or the Hco-acr-8 gene ortholog upstream of the indel (for example a primer having the nucleic acid sequence of SEQ ID NO: 16) and wherein the second primer has a nucleic acid sequence corresponding to a second location in the Hco-acr-8g gene or the Hco-acr-8 gene ortholog downstream of the indel (for example a primer having the nucleic acid sequence of SEQ ID NO: 17). In still another embodiment, the method further comprises, in the determination step, (i) contacting the genomic DNA with at least a third primer specific for the vicinity of the indel and a fourth primer specific for the indel under conditions to form a complex between the genomic DNA when the indel is present, and (ii) detecting the presence of the complex; and/or, in the characterization step, characterizing the nematode as susceptible to the imidazothiazole anti-helminthic when the complex is present and as resistant to the imidazothiazole anti-helminthic when the complex is absent. In yet another embodiment, the method further comprises, in the determination step, (iii) amplifying the genomic DNA with the third primer and the fourth primer to provide at least one amplicon, and (iv) determining the presence of the complex based on the formation of the at least one amplicon. In a further embodiment, the third primer is specific for a location upstream or downstream of the indel. In yet another embodiment, the method further comprises, in the determination step, contacting the genomic DNA with at least one probe specific for the indel located in the Hco-acr-8 gene or the Hco-acr-8 gene ortholog under conditions to form a complex between the genomic DNA and the probe when the indel is present and (ii) detecting the presence of the complex; and/or in the characterization step, characterizing the nematode as susceptible to the imidazothiazole anti-helminthic when the complex is detected and as resistant to the imidazothiazole anti-helminthic when the complex is not detected. In another embodiment, the method further comprises, in the determination step, (i) amplifying the Hco-acr-8 gene, the Hco-acr-8 gene ortholog or a fragment thereof to provide at least one amplicon and (ii) contacting the probe with the at least one amplicon so as to form a complex when the indel is present; and/or in the characterization step, characterizing the nematode as susceptible to the imidazothiazole anti-helminthic when the complex is determined to be present in the at least one amplicon and as resistant to the imidazothiazole anti-helminthic when the complex is determined to be absent from the at least one amplicon. In still another embodiment, the method further comprises, in the determination step, extracting an identity of a nucleic acid base at a plurality of positions along the Hco-acr-8 gene, the Hco-acr-8 gene ortholog or a fragment thereof and comparing with the identity of at least one nucleic acid base of the indel at corresponding positions.

According to a second aspect, the present disclosure provides an isolated nucleic molecule consisting essentially of the nucleic acid sequence of SEQ ID NO: 9, an oligonucleotide having at least 10 consecutives nucleic acid bases of SEQ ID NO: 9 (which can optionally be used as a primer) as well as an oligonucleotide having at least 30 consecutive nucleic acid bases of SEQ ID NO: 9 (which can optionally be used as a probe). In an embodiment, the nucleic acid residue at position 4 of SEQ ID NO: 9 is T and/or the nucleic acid residue at position 61 of SEQ ID NO: 9 is C.

According to a third aspect, the present disclosure provides a commercial package for the detection of resistance to an imidazothiazole anti-helminthic. The commercial package comprises means for determining the presence or absence of an indel having the sequence of SEQ ID NO: 9 and being located in the a Hco-acr-8 gene or a Hco-acr-8 gene ortholog and instructions for characterizing the resistance of the nematode to the imidazothiazole anti-helminthic based on the presence or absence of the indel. In an embodiment, the means for determining the presence or absence of the indel comprise a probe specific for the indel. In another embodiment, the means for determining the presence or absence of the indel comprise a pair of primers specific for amplifying the indel or a fragment thereof. In an embodiment, the nucleic acid residue at position 4 of SEQ ID NO: 9 is T and/or the nucleic acid residue at position 61 of SEQ ID NO: 9 is C.

Throughout the description of the present disclosure, several terms are used that are specific to the science of this field. For the sake of clarity and to avoid any misunderstanding, these definitions are provided to aid in the understanding of the specification and claims.

Allele. The term “allele” refers to one of a pair, or series, of forms of a genetic region that occur at a given locus in a chromosome. An “associated allele” refers to a specific allele at a polymorphic locus that is associated with a particular phenotype of interest, e.g., a predisposition to a disorder or a particular response to an agent. Within a population, given multiple loci, there may be more than one combination of alleles associated with a phenotype of interest.

Amplification. As used herein, the terms “amplification”, “amplifying” and the like refer generally to any process that results in an increase in the copy number of a nucleic acid molecule or set of related nucleic acid molecules. As it applies to polynucleotide molecules, amplification means the production of multiple copies of a polynucleotide molecule, or a portion of a polynucleotide molecule, typically starting from a small amount (undetectable without amplification) of a polynucleotide, until, typically, the amplified material becomes detectable by spectroscopic, photochemical, biochemical, immunochemical, chemical, or other detection. Amplification of polynucleotides encompasses a variety of chemical and enzymatic processes. The generation of multiple DNA copies from one or a few copies of a template DNA molecule during a polymerase chain reaction (PCR), a strand displacement amplification (SDA) reaction, a transcription mediated amplification (TMA) reaction, a nucleic acid sequence-based amplification (NASBA) reaction, or a ligase chain reaction (LCR) are forms of amplification. Amplification is not limited to the strict duplication of the starting molecule. For example, the generation of multiple cDNA molecules from a limited amount of RNA in a sample using RT-PCR is a form of amplification. Furthermore, the generation of multiple RNA molecules from a single DNA molecule during the process of transcription is also a form of amplification.

Antibody. As used herein, an “antibody” include monoclonal antibodies (including full length monoclonal antibodies), polyclonal antibodies, multispecific antibodies (e.g. bispecific antibodies), single domain antibodies and antibody fragments. Examples of antibody fragments include, but are not limited to, Fab, Fab′, F(ab′)₂, and Fv fragments; diabodies; linear antibodies; single-chain antibody molecules; and multi-specific antibodies formed from antibody fragments. The term “antibody” may also include chimeric or humanized antibodies.

Naturally occurring immunoglobulins have a common core structure in which two identical light chains (about 24 kD) and two identical heavy chains (about 55 or 70 kD) form a tetramer. The amino-terminal portion of each chain is known as the variable (V) region and can be distinguished from the more conserved constant (C) regions of the remainder of each chain. Within the variable region of the light chain is a C-terminal portion known as the J region. Within the variable region of the heavy chain, there is a D region in addition to the J region. Most of the amino acid sequence variation in immunoglobulins is confined to three separate locations in the V regions known as hypervariable regions or complementarity determining regions (CDRs) which are directly involved in antigen binding. Proceeding from the amino-terminus, these regions are designated CDR1, CDR2 and CDR3, respectively. The CDRs are held in place by more conserved framework regions (FRs). Proceeding from the amino-terminus, these regions are designated FR1, FR2, FR3, and FR4, respectively.

Antibody derivatives include, but are not limited to, humanized antibodies. As used herein, the term “humanized antibody” refers to an immunoglobulin that comprises both a region derived from a human antibody or immunoglobulin and a region derived from a non-human antibody or immunoglobulin. The action of humanizing an antibody consists in substituting a portion of a non-human antibody with a corresponding portion of a human antibody. For example, a humanized antibody as used herein could comprise a non-human variable region (such as a region derived from a murine antibody) capable of specifically recognizing a polypeptide encoded by a gene as described herein and a human constant region derived from a human antibody. In another example, the humanized immunoglobulin can comprise a heavy chain and a light chain, wherein the light chain comprises a complementarity determining region derived from an antibody of non-human origin which binds to the polypeptide and a framework region derived from a light chain of human origin, and the heavy chain comprises a complementarity determining region derived from an antibody of non-human origin which binds to the polypeptide and a framework region derived from a heavy chain of human origin.

As used herein, the present disclosure also relates to fragments of the antibodies. “Antibody fragments” comprise a portion of a full length antibody, generally the antigen binding or variable region thereof. As used herein, a “fragment” of an antibody (e.g. a monoclonal antibody) is a portion of an antibody that is capable of specifically recognizing the same epitope as the full version of the antibody. In the present patent disclosure, antibody fragments are capable of specifically recognizing the polypeptide. Antibody fragments include, but are not limited to, the antibody light chain, single chain antibodies, Fv, Fab, Fab′ and F(ab′)₂ fragments. Such fragments can be produced by enzymatic cleavage or by recombinant techniques. For instance, papain or pepsin cleavage can be used to generate Fab or F(ab′)₂ fragments, respectively. Antibodies can also be produced in a variety of truncated forms using antibody genes in which one or more stop codons have been introduced upstream of the natural stop site. For example, a chimeric gene encoding the heavy chain of an F(ab′)₂ fragment can be designed to include DNA sequences encoding the CH1 domain and hinge region of the heavy chain. Antibody fragments can also be humanized. For example, a humanized light chain comprising a light chain CDR (i.e. one or more CDRs) of non-human origin and a human light chain framework region. In another example, a humanized immunoglobulin heavy chain can comprise a heavy chain CDR (i.e., one or more CDRs) of non-human origin and a human heavy chain framework region. The CDRs can be derived from a non-human immunoglobulin.

Hco-acr-8 gene and Hco-acr-8 gene ortholog. The Hco-acr-8 gene encodes the Hco-ACR-8 or HAX protein which is a subunit of the levamisole sensitive acetylcholine receptor of Haemonchus contortus. In the context of the present disclosure, a “Hco-acr-8 gene ortholog” is understood to be a gene in a different species that evolved from a common ancestral gene by speciation. In the context of the present disclosure, a Hco-acr-8 gene ortholog retains the same function, e.g. it can act as a subunit of the levamisole sensitive acetylcholine receptor.

Identity. The term “identity”, as known in the art, refers to a relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. Identity and similarity can be readily calculated by known methods, including but not limited to those described in A. M. Lesk (ed), 1988, Computational Molecular Biology, Oxford University Press, NY; D. W. Smith (ed), 1993, Biocomputing. Informatics and Genome Projects, Academic Press, NY; A. M. Griffin and H. G. Griffin, H. G (eds), 1994, Computer Analysis of Sequence Data, Part 1, Humana Press, NJ; G. von Heinje, 1987, Sequence Analysis in Molecular Biology, Academic Press; and M. Gribskov and J. Devereux (eds), 1991, Sequence Analysis Primer, M Stockton Press, NY; H. Carillo and D. Lipman, 1988, SIAM J. Applied Math., 48:1073.

A nucleic acid molecule or fragment thereof is “substantially identical” or “substantially homologous” to another if, when optimally aligned (with appropriate nucleotide insertions and/or deletions) with the other nucleic acid (or its complementary strand), there is nucleotide sequence identity in at least 60% of the nucleotide bases, usually at least 70%, more usually at least 80%, preferably at least 90%, and more preferably at least 95-98% of the nucleotide bases. Alternatively, substantial homology or substantial identity exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least about 55% sequence identity over a stretch of at least about nine or more nucleotides, preferably at least about 65%, more preferably at least about 75%, and most preferably at least about 90%. The length of homology or identity comparison, as described, may be over longer stretches, and in certain embodiments will often be over a stretch of at least 5 nucleotides, at least 14 nucleotides, at least 20 nucleotides, more usually at least 24 nucleotides, typically at least 28 nucleotides, more typically at least 32 nucleotides, and preferably at least 36 or more nucleotides.

Indel. In the context of the present disclosure, the term indel refers to a nucleic acid molecule which may be present (e.g. inserted) or absent (e.g. deleted) from a region between exon 2 and 3 of the Hco-acr-8 gene (or its ortholog). In an embodiment, the indel is at most 63 nucleotides long and has a sequence substantially identical to the one presented in SEQ ID NO: 9 and reproduced below in FIG. 4:

3′-tttngacttg atgttttgtt aactgctgtt atatcgccgc agtacgcgta aggctgatta ntg-5′ (SEQ ID NO: 9)

FIG. A. Potential ucleic acid sequence of the indel. At position 4, the residue can be G or T. At position 61, the residue can be Y or C. In an embodiment of the indel, when the residue at position 4 is a G, the residue at position 61 is a Y (e.g., a pyrimidine, such as T or C). In another embodiment of the indel, when the residue at position 4 is a G, the residue at position 61 is a C. In a further embodiment, when the residue at position 4 is a G, the residue at position 61 is a T. In an embodiment of the indel, when the residue at position 4 is a T, the residue at position 61 is a Y (e.g., a pyrimidine, such as T or C). In another embodiment of the indel, when the residue at position 4 is a T, the residue at position 61 is a C. In a further embodiment, when the residue at position 4 is a T, the residue at position 61 is a T.

As shown herein, the presence or absence of this indel is associated with sensitivity or resistance to an imidazothiazole anti-helmintic.

Nucleic acid. As used herein, “nucleic acid”, “nucleotide sequence” or “nucleic acid molecule” refer to a polymer of DNA and/or RNA which may be single or double stranded and optionally containing synthetic, non-natural or altered nucleotide bases capable of incorporation into DNA or RNA polymers. “Nucleic acid”, “nucleotide sequence” or “nucleic acid molecule” may encompass genes, cDNA, DNA (e.g. genomic DNA) and RNA encoded by a gene. Nucleic acids or nucleic acid sequences may comprise at least 3, at least 10, at least 100, at least 1000, at least 5000, or at least 10000 nucleotides or base pairs. “Nucleic acid”, “nucleotide sequence” or “nucleic acid molecule” may be modified by any chemical and/or biological means known in the art including, but not limited to, reaction with any known chemicals such as alkylating agents, browning sugars, etc; conjugation to a linking group; methylation; oxidation; ionizing radiation; or the action of chemical carcinogens. Such nucleic acid modifications may occur during synthesis or processing or following treatment with chemical reagents known in the art. Probes, oligonucleotides and primers can be made from nucleic acid bases or modified nucleic acid bases.

As used herein, “consists essentially of” or “consisting essentially of” means that the nucleic acid sequence may include one or more nucleotide bases, including within the sequence or at one or both ends of the sequence, but that the additional nucleotide bases do not materially affect the function of the nucleic acid sequence.

An “isolated nucleic acid molecule” may refer to a nucleic acid molecule that does not occur in nature as part of a larger polynucleotide sequence; and/or may be substantially free from any other nucleic acid molecules or other contaminants that are found in its natural environment. As used herein, an “isolated nucleic acid molecule” may also encompass recombinantly or synthetically produced nucleic acid molecules. For example, a synthetic copy of a genomic DNA sequence is considered to be an isolated nucleic acid molecule.

Nucleic acids also specifically includes a molecule or a collection of molecules isolated from the genome of the nematode. Such molecules are collectively referred to as the genomic DNA of the nematode.

Peptide. As used herein, the terms “peptide”, “oligopeptide”, “polypeptide” and “protein” may be used interchangeably and encompasses any chain of naturally or non-naturally occurring amino acids (either D- or L-amino acids), regardless of length (e.g. at least 5, 6, 8, 10, 12, 14, 16, 18, 20, 25, 30, 40, 50, 100 or more amino acids) or post-translational modification (e.g., glycosylation or phosphorylation) or the presence of e.g. one or more non-amino acyl groups (for example, sugar, lipid, etc.) covalently linked to the peptide, and includes, for example, natural proteins, synthetic or recombinant polypeptides and peptides, hybrid molecules, peptoids, peptidomimetics, etc. Peptides may also be monomeric or multimeric. Peptide fragments may comprise a contiguous span of at least 5, at least 10, at least 25, at least 50, at least 100, at least 250, at least 500, at least 1000, at least 1500, or at least 2500 consecutive amino acids and may retain the desired activity of the full length peptide.

Peptide mimetics. Peptide mimetics mimic the three-dimensional structure of a polypeptide. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others. In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

Polymerase chain reaction. As used herein, the term “polymerase chain reaction” (PCR) refers to a method for amplification well known in the art for increasing the concentration of a segment or a fragment of a target polynucleotide in a sample, where the sample can be a single polynucleotide species, or multiple polynucleotides. Generally, the PCR process consists of introducing a molar excess of two or more extendable oligonucleotide primers to a reaction mixture comprising the desired target sequence(s), where the primers are complementary to opposite strands of the double stranded target sequence. The use of the primers enable the production of amplicons that represent a target or standard sequence. The reaction mixture is usually subjected to a program of thermal cycling in the presence of a DNA polymerase, resulting in the amplification of the desired target sequence flanked by the DNA primers. Reverse transcriptase PCR (RT-PCR) is a PCR reaction that uses an RNA template and a reverse transcriptase, or an enzyme having reverse transcriptase activity, to first generate a single stranded DNA molecule prior to the multiple cycles of DNA-dependent DNA polymerase primer elongation. Multiplex PCR refers to PCR reactions that produce multiple copies of more than one product or amplicon in a single reaction, typically by the inclusion of more than two different primers in a single reaction.

Resistance/Susceptibility to an imidazothiazole anti-helmintic. As used in the context of the present disclosure, a nematode is said to be resistant to an imidazothiazole anti-helmintic if less than about 95%, less than about 93%, less than about 91%, less than about 89%, less than about 87%, less than about 85%, less than about 83%, less than about 81%, less than about 79%, less than about 77%, less than about 75%, less than about 73%, less than about 71%, less than about 69%, less than about 67%, less than about 65%, less than about, 63%, less than about 61%, less than about 59%, less than about 57%, less than about 55%, less than about 53%, less than about 51%, less than about 49%, less than about 47%, less than about 45%, less than about 43%, less than about 41%, less than about 39%, less than about 37%, less than about 35%, less than about 33%, less than about 31%, less than about 29%, less than about 27%, less than about 25%, less than about 23%, less than about 21%, less than about 19%, less than about 17%, less than about 15%, less than about 13%, less than about 11%, less than about 9% or less than about 7% of nematodes die following exposure to a LD₉₅ dose or concentration of an imidazothiazole anti-helmintic (Coles et al., 2006). On the other hand, a nematode is said to be sensitive to an imidazothiazole anti-helmintic if at most about 5%, at most about 4%, at most about 3%, at most about 2%, at most about 1% or if 0% of nematodes survive following exposure to a LD₉₅ dose or concentration of an imidazothiazole anti-helmintic (Coles et al., 2006).

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates Hco-ACR-8 subunit synthesis (black lines) and Hco-ACR-8b truncated form synthesis (dashed lines). The bold underscore lines represent the regions that were amplified and sequenced. The region A refers to the end of exon 2 and the signal GT, the region B refers to region upstream of exon 3b and contains the AG splice site and the YYRAY putative splicing branch point. The region C contains the beginning of exon 3 and the boundary site AG.

FIG. 2 illustrates the organization of the Hco-acr-8 gene. The exon 3b is the sequence recruited to build the truncated isoform Hco-acr-8b and is present in the intron 2 of the Hco-acr-8 gene. The indel of 63 bp is represented in grey in the sequence of exon 3b and is shown by the arrow.

FIG. 3 illustrates the different sizes of amplification products from various individuals and pools from different isolates. The lower band does not possess the indel; the upper band possesses the indel. Some individuals like RHS6-5, RHS6-6 and ISE-9 appeared to be heterozygous for the presence of the 63 bp insertion.

FIG. 4 provides the alignment of the sequences of exon 3b, copy 1092 and copy 1478. The copy 1092 (945 bp) does not include the sequence of exon 3b (684 bp) as shown by the alignment the two sequences which do not overlap. However, the copy 1478 (>4000 bp) includes the sequence of exon 3b and can be used in the study of allelic variation of Hco-acr-8.

FIG. 5 illustrates the electrophoresis gel for two individual males each from Kokstad, Cedara, RHS6, Zaire and ISE isolates and pools of 50 larvae from UGA/2004 isolate. A first PCR was performed on individuals and pools with the primers specific of copies 1478, 2346 and 2831. The PCR products of this first PCR were used as templates for a nested PCR re-amplification to amplify the indel area. At the same time, another PCR was run on DNA extracts with the primers for the indel area. On the gels, “DNA extract” indicates the single amplification with the DNA extract as template and “1478”, “2346” and “2831” indicate the amplifications after a PCR with primers specific for the copy followed by a nested PCR to amplify the region with the indel.

FIG. 6 provides the visualization of β-tubulin transcripts and Hco-acr-8b (HAX) transcripts for the isolates PF, CRA, ISE, Roggliswil (ROG), Courtion (COUR) and Kokstad (KOK) on the electrophoresis gel. The arrows indicate the amplification of Hco-acr-8b (HAX) transcript. The isolate CRA produce a light amplification of the Hco-acr-8b (HAX) transcript.

FIG. 7 illustrates the presence or absence of the insertion of 63 bp in the isolates PF, CRA, ISE, Roggliswil (ROG), Courtion (COUR) and Kokstad (KOK) on the electrophoresis gel. The arrows indicate the two possible amplicon sizes: 319 bp indicates the presence of the insertion and the 256 bp band indicates the absence of the insertion. The isolate CRA is levamisole susceptible but shows a weak band with the insertion (319 bp).

FIG. 8 illustrates the boundary region: GT splice variant site at the beginning of intron 2 is enclosed in a black box. The identification of individuals and pools is on the left of the alignment. All the parasites sequenced in the study had a GT at the beginning of intron 2. Results are shown for Kokstad 1 (SEQ ID NO: 18), Kokstad 5 (SEQ ID NO: 19), Kokstad 6 (SEQ ID NO: 20), Kokstand 8 (SEQ ID NO: 21), Cedara 4 (SEQ ID NO: 22), Cedara 5 (SEQ ID NO: 23), Cedera 6 (SEQ ID NO: 24), Cedara 8 (SEQ ID NO: 25), RHS6-2 (SEQ ID NO: 26), RHS6-3 (SEQ ID NO: 27), RHS6-5 (SEQ ID NO: 28), RHS6-6 (SEQ ID NO: 29), RHS6-8 (SEQ ID NO: 30), UGA—pool 1 (SEQ ID NO: 31), UGA—pool 3 (SEQ ID NO: 32), ISE 4 (SEQ ID NO: 33), ISE 5 (SEQ ID NO: 34), ISE 8 (SEQ ID NO: 35), PF3 (SEQ ID NO: 36), PF9 (SEQ ID NO: 37), PF10 (SEQ ID NO: 38), Zaire 2 (SEQ ID NO: 39), Zaire 4 (SEQ ID NO: 40), Zaire 5 (SEQ ID NO: 41), Zaire 7 (SEQ ID NO: 42), Zaire 9 (SEQ ID NO: 43), CRA6-r (SEQ ID NO: 44), CRA8-r (SEQ ID NO: 45), ROG—pool 1 (SEQ ID NO: 46), Cour—pool 1 (SEQ ID NO: 47), Cour—pool 2 (SEQ ID NO: 48) and Cour—pool 3 (SEQ ID NO: 49).

FIG. 9 illustrates the boundary region: AG splice variant site at the end of intron 2 is enclosed in a black box. The identification of individuals and pools is on the left of the alignment. All the parasites sequenced in the study had an AG at the end of intron 2. Results are shown for Kokstad 1 (SEQ ID NO: 50), Kokstad 5 (SEQ ID NO: 51), Kokstad 6 (SEQ ID NO: 52), Kokstand 8 (SEQ ID NO: 53), Cedara 3 (SEQ ID NO: 54), Cedara 4 (SEQ ID NO: 55), Cedara 5 (SEQ ID NO: 56), Cedara 8 (SEQ ID NO: 57), RHS6-2 (SEQ ID NO: 58), RHS6-3 (SEQ ID NO: 59), RHS6-5 (SEQ ID NO: 60), RHS6-6 (SEQ ID NO: 61), RHS6-8 (SEQ ID NO: 62), CRA4 (SEQ ID NO: 63), CRA5 (SEQ ID NO: 64), CRA8 (SEQ ID NO: 65), CRA9 (SEQ ID NO: 66), ISE 5 (SEQ ID NO: 67), ISE 7 (SEQ ID NO: 68), ISE 9 (SEQ ID NO: 69), ISE 10 (SEQ ID NO: 70), PF1 (SEQ ID NO: 71), PF3 (SEQ ID NO: 72), PF6 (SEQ ID NO: 73), PF7 (SEQ ID NO: 74), PF10 (SEQ ID NO: 75), Zaire 5 (SEQ ID NO: 76), Zaire 7 (SEQ ID NO: 77) and Zaire 8 (SEQ ID NO: 78).

FIG. 10 illustrates the AG splice variant site at the beginning of exon 3b is enclosed in a black box. The identification of individuals and pools is on the left of the alignment. All the parasites sequenced in the study had an AG at the beginning of exon 3b. Results are shown for Kokstad 1 (SEQ ID NO: 79), Kokstad 4 (SEQ ID NO: 80), Kokstad 5 (SEQ ID NO: 81), Kokstad 7 (SEQ ID NO: 82), Cedara 3 (SEQ ID NO: 83), Cedara 4 (SEQ ID NO: 84), Cedara 5 (SEQ ID NO: 85), Cedara 8 (SEQ ID NO: 86), RHS6-1 (SEQ ID NO: 87), RHS6-2 (SEQ ID NO: 88), RHS6-5 (SEQ ID NO: 89), RHS6-6 (SEQ ID NO: 90), RHS6-8 (SEQ ID NO: 91), RHS6-9 (SEQ ID NO: 92), Howick 1 (SEQ ID NO: 93), Howick 3 (SEQ ID NO: 94), Howick 5 (SEQ ID NO: 95), UGA—pool 2 (SEQ ID NO: 96), UGA—pool 3 (SEQ ID NO: 97), Zaire 1 (SEQ ID NO: 98), Zaire 3 (SEQ ID NO: 99), Zaire 4 (SEQ ID NO: 100), Zaire 6 (SEQ ID NO: 101), Zaire 7 (SEQ ID NO: 102), ISE 5 (SEQ ID NO: 103), ISE 6 (SEQ ID NO: 104), ISE 7 (SEQ ID NO: 105), ISE 8 (SEQ ID NO: 106), ISE 9 (SEQ ID NO: 107), CRA 4 (SEQ ID NO: 108), CRA 6 (SEQ ID NO: 109), CRA 7 (SEQ ID NO: 110) and CRA 9 (SEQ ID NO: 111).

FIG. 11 illustrates the branch point sequences YYRAY present in different isolate sequences. The YYRAY sequences are indicated with a grey triangle. All individuals from Kokstad, Cedara, RHS6, Howick, Zaire, ISE and PF were grouped as one sequence to not overcharge the alignment. So were the sequences of the two pools of each of UGA/2004 and CRA.

FIG. 12 illustrates the alignment of the three copy sequences and the sequence of exon 3b. The sequence of exon 3b overlaps the three sequences of copies 1478, 2346 and 2831. The three copies include the sequence of exon 3b and can be used to study the allelic variation of Hco-acr-8 gene.

### DETAILED DESCRIPTION

In accordance with the present disclosure, there is provided a method of detecting an imidazothiazole anti-helmintic resistance of nematodes as well as tools and commercial packages to perform the method. The method is based on the determination of the presence or absence of an indel (in an embodiment, an indel having or consisting essentially of the sequence of SEQ ID NO: 9) in the Hco-acr-8 gene (or a corresponding gene ortholog). As it will be further discussed below, the presence of the indel is more frequently associated with nematodes being sensitive to an imidazothiazole anti-helmintic as well as expressing a full-length transcript of the Hco-acr-8 gene (or gene ortholog) encoding a functional HAX protein. As it will also be discussed below, the absence of the indel is more frequently associated with nematodes being resistant to an imidazothiazole anti-helmintic as well as expressing a truncated transcript of the Hco-acr-8 gene (or gene ortholog) encoding a non-functional HAX protein. Consequently, by determining the presence or absence of the indel in the Hco-acr-8 gene, it is possible to assess the likelihood of resistance to an imidazothiazole anti-helmintic in nematodes and/or the expression of a functional HAX protein in these nematodes. For example, homozygotes for the presence of the indel are more frequently associated with susceptibility to an imidazothiazole anti-helmintic whereas homozygotes for the absence of the indel are more frequently associated with resistance to an imidazothiazole anti-helmintic. It is also believed that heterozygotes for the indel show a loss in susceptibility and are considered resistant or partially resistant.

The methods and commercial packages described herewith can be used with any imidazothiazole anthelmintics, including levamisole and tetramisole. As it is known in the art, tetramisole is a racemic mixture of (S)-6-phenyl-2,3,5,6-tetrahydroimidazo[2,1-b][1,3]thiazole, whereas levamisole is the L-racemer. Since there is a connection between the levamisole receptor and the tetrahydropyrimidine receptor, it is believed that there is a level of cross resistance between levamisole and tetrahydropyrimidine anti-helminthic. Consequently, in some embodiment, the method can also be used to determine the susceptibility/resistance of tetrahydropyrimidine anti-helminthic (e.g. pyrantel, morantel, oxantel) based on the characterization of the Hco-acr-8 gene (or it corresponding ortholog).

As will be shown below, the genomic sequence of the Hco-acr-8 gene surrounding the indel was analyzed for the possible truncated transcript splice sites from many different field isolates of H. contortus that were either susceptible or an imidazothiazole anti-helmintic resistant. Various isolates were screened and included Kokstad, Cedara, RHS6, UGA/2004 and Zaire for which an imidazothiazole anti-helmintic resistance status and presence or absence of the truncated form had already been demonstrated. In addition, isolates from diverse geographic origins where the an imidazothiazole anti-helmintic resistance/susceptibility status was known were also characterized. Sequences of the region including exon 2, intron 2, exon 3b, and the beginning of exon 3 were examined. The results shown herein indicate that the presence of an indel between exon 2 and 3 (in a region referred to as exon 3b) is associated with susceptibility to an imidazothiazole anti-helmintic whereas the absence of such indel is associated with resistance to an imidazothiazole anti-helmintic.

### Methods for Detecting an Imidazothiazole Anti-Helmintic Resistance

The first step for determining susceptibility/resistance to an imidazothiazole anti-helmintic is to obtain a genomic DNA sample from a nematode. The genomic DNA can be obtained from an in vitro culture of the nematode. The genomic DNA can also be obtained from a biological sample of a subject at least suspected of being infected by the parasite. The subject may be, without limitation, humans, livestock (such as, for example, cattle and other ruminants (including sheep and goats)), pigs and fish. In the context of the present disclosure, a biological sample may be any sample (e.g. bodily fluid, excrement, organ, tissue, etc) from a subject. In an embodiment, the sample is a stool sample containing at least one nematode egg. The nematodes of the biological sample can be optionally expanded in vitro prior to the isolation of the genomic DNA. For example, the nematode egg can be expanded in vitro prior to the isolation of the genomic DNA by being cultured under conditions so as to allow at least one nematode larva to hatch.

Since the methods described herein are based on the detection of alterations in the genomic DNA of the nematodes (and not transcripts or proteins), the methods are not limited to the characterization of adult nematodes. The methods can successfully applied to nematodes at the larval stage (L1, L2, L3 or L4 stages) and even a nematode egg.

In the detection methods described herein, the genomic DNA sample (optionally purified) or a synthetic copy (obtained by nucleic acid amplification for example) of the genomic DNA sample can be used. Methods of isolating nucleic acids from nematodes and biological samples are known. Such methods may include, but are not limited to, traditional DNA extraction, with proteinase K digestion followed by phenol chloroform extraction, sodium hydroxide extraction, and physical disruption, followed by purification, e.g. by cesium chloride centrifugation or high performance liquid chromatography (HPLC); or the use of commercial kits, e.g. QIAamp™ or DNeasy™. A skilled person would appreciate that different approaches may be used to isolate a nucleic acid sample from a nematode. In an embodiment of the disclosure, the nucleic acid sample comprises genomic DNA.

Once the genomic DNA is obtained (and optionally a synthetic copy of such genomic DNA has been made), it must be determined if an indel is present or absent in the Hco-arc-8 gene (or its corresponding gene ortholog). In an embodiment, the indel has or consists essentially of the nucleic acid sequence as shown in SEQ ID NO: 9. When present, the indel is preferably located between exon 2 and 3 of the Hco-arc-8 gene (or its corresponding ortholog) in a region referred to as exon 3b.

In an embodiment, the determination of the presence or absence of the indel can be made by an individual. In another embodiment, the comparison can be made in a determination module. Such determination module may comprise a processor and a memory card to perform an application. The processor may access the memory to retrieve data. The processor may be any device that can perform operations on data. Examples are a central processing unit (CPU), a front-end processor, a microprocessor, a graphics processing unit (PPU/VPU), a physics processing unit (PPU), a digital signal processor and a network processor. The application is coupled to the processor and configured to determine the presence or absence of the indel. An output of this determination may be transmitted to a display device. The memory, accessible by the processor, receives and stores data, or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

As indicated above, the presence of the indel allows the formation of a full-length transcript of the Hco-acr-8 gene (or gene ortholog), the expression of a functional HAX protein and is preferably associated with a phenotype of susceptibility to levamisole. As also indicated above the absence of the indel is associated with the formation of a truncated transcript (sometimes referred to as the Hco-arc-8b isoform) of the Hco-acr-8 gene (or gene ortholog), the expression of a non-functional HAX protein and is preferably associated with a phenotype of resistance to levamisole. As such, the presence or absence of the indel allows for the characterization of the phenotype of the nematode with respect to levamisole resistance. The nematode is considered susceptible to an imidazothiazole anti-helmintic when the indel is determined to be present in the genomic DNA sample (or products or copies derived therefrom). Alternatively, the nematode is considered resistant to an imidazothiazole anti-helmintic when the indel is determined to be absent from the genomic DNA sample (or products derived therefrom).

In an embodiment, the characterization can be made by an individual. In another embodiment, the characterization can be made with a processor and a memory card to perform an application. The processor may access the memory to retrieve data. The processor may be any device that can perform operations on data. Examples are a central processing unit (CPU), a front-end processor, a microprocessor, a graphics processing unit (PPU/VPU), a physics processing unit (PPU), a digital signal processor and a network processor. The application is coupled to the processor and configured to characterize the nematode being tested. An output of this characterization may be transmitted to a display device. The memory, accessible by the processor, receives and stores data. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

The method described herein can be applied to any nematode having the Hco-acr-8 gene (or its corresponding gene ortholog). In an embodiment, the method is practiced on nematodes of the Trichostrongylidae family (such as, for example, from the genus Haemonchus, and in still another example, from the species Haemonchus contortus).

As indicated above, prior to the determination of the presence or absence of the indel, in some embodiments, a nucleic-acid copy or amplification of the genomic DNA of the nematode can be made. Optionally, the nucleic-acid copy of the genome is generated via an amplification step which provides multiple copies of the genomic DNA (or portions thereof). The nucleic-acid copy can be a copy of the entire genome of the nematode or only a portion of the genome of the nematode. When the nucleic-acid copy of a portion of the genome of the nematode is made, it is important that it comprises the region of the Hco-acr-8 gene susceptible of comprising the indel, especially the region between exon 2 and exon 3 (e.g., exon 3b for example).

The methods presented herein can also be used to monitor imidazothiazole anti-helminthic resistance in a population of nematodes. In order to do so, the method is practiced at a first point in time to determine, if any, the presence of imidazothiazole resistance in a population of nematodes. Then, the method is performed at least a second time, later in time, in order to determine if the phenotype of the population has changed. In an embodiment, imidazothiazole can be administered to the population of nematodes between the first and second point in time.

The methods presented herein can also be used to determine if a subject intended to be treated with an imidazothiazole anti-helmintic or already being treated with an imidazothiazole anti-helmintic can benefit from an imidazothiazole anti-helmintic treatment. In order to do so, a biological sample from the subject suspected or known to be afflicted with a nematode infection is obtained either prior to an imidazothiazole anti-helmintic administration or after the administration of at least one dose of an imidazothiazole anti-helmintic. The genomic DNA of the nematodes contained in the biological samples is isolated and analyzed to determine if the indel is present or absent. The presence of the indel indicates that the subject will benefit from an imidazothiazole anti-helmintic treatment (because the infecting nematodes are considered susceptible to an imidazothiazole anti-helmintic). In an embodiment, the method can also encompass administering an imidazothiazole anti-helmintic in such subjects. On the other hand, the absence of the indel indicates that the subject will not benefit from an imidazothiazole anti-helmintic treatment (because the infecting nematodes are considered resistant to an imidazothiazole anti-helmintic). In an embodiment, the method can also encompassing avoiding or discontinuing an imidazothiazole anti-helmintic treatment in such subject.

The methods presented herein can also be used to monitor an imidazothiazole anti-helmintic treatment in the treated subject and determine the predisposition of resistance in the treated subject.

In an embodiment, the presence or absence of the indel can be made via nucleic acid amplification and the characterization of the amplification products.

The detection of the indel can be made using various assays based on the amplification of the genomic DNA, optionally in combination with the hybridization of a probe to the amplicon. Some methods involve temperature specific annealing of a primer or a probe to the target sequence containing the indel. When both primer and target are derived from wild type sequences or both from the mutant sequence the annealing temperature is usually several degrees higher than when one comes from the mutant and the other from wild type. Small differences in annealing temperature between heterologous and homologous primer and target pairs can be enhanced by using locked nucleic acids at the indel site or minor groove binders nearby. These differences can be visualized, for example, in Invader Assays™, or with TaqMan, Molecular Beacon, or fluorescent resonant energy transfer (FRET) probes by performing the detection step at a temperature in the window between the homologous and heterologous annealing temperatures. Allele specific PCR or ligation can be used with primers with the indel (or part thereof) at the 3′ end of the primer. When the indel form part of a naturally occurring or artificially engineered restriction endonuclease site, digestion of the amplicon with such enzymes can reveal the presence of the indel. The melting temperature of FRET probes or the high resolution melting (HRM) profiles of short amplicons can also infer sequence data revealing the indel. In optional embodiments, amplicons can be sequenced, pyrosequenced, or hybridized to specific probes.

High resolution melting or HRM is a method based on PCR amplification of a short sequence and the use of a double-stranded specific fluorescent dye and can be used for the detection of the indel. Briefly, the sequence is amplified and the dye inserts within the double-stranded amplicon. Following PCR amplification, a very slow temperature ramping induced amplicon melting is performed and fluorescence (or loss thereof) is measured. Since each DNA amplicon possess a unique denaturation pattern and the presence of a polymorphism (such as an indel) can modify the amplicon's denaturation pattern, high resolution melting can be used to distinguish between sequences differing in their nucleotide sequence.

In an embodiment of the amplification methods described herein, the isolated genomic DNA (or a synthetic copy thereof) can be combined and contacted with at least one pair of primers specific for the region where the indel is located in the Hco-acr-8 gene (or its corresponding gene ortholog) under conditions to form a complex between the genomic DNA and the pair of primers. In this example, each pair of primers are located upstream and downstream of the region that can encompass the indel so as to amplify, if any, the indel (as well as neighboring sequences). In an embodiment, the region encompassed by the pair of primers is limited to the Hco-acr-8 gene (or its corresponding ortholog). In such example, the genomic DNA of all nematodes tested will form a complex with the pair of primers, irrespective of the presence or absence of the indel. A nucleic-acid polymerization step is then conducted and the resulting amplification products are characterized to determine the presence or absence of the indel. The characterization can be made by determining the sequence identity of the amplification products to assess if the sequence of the indel is present or absent from the amplification products. Alternatively or complementarily, the characterization can be made by determining the size of the amplification product to assess if the indel is present or absent in the amplification products. It is believed that the presence of the indel will generate larger amplification products. Such size characterization can be made visually. In this example, a further confirmatory step of using a probe specific for the indel can also be used to characterize the amplification products (and ultimately determine the presence or absence of the indel).

In the embodiment in which primers are used to amplify the indel (or lack thereof) in its entirety and are located both upstream and downstream of the position of the indel, the primers are designed in order to preferably locate in a region (e.g., between 20 and 25 nucleic acid base-long) of high identity between the different nematode strains. Further, in an embodiment, the primers can be designed to generate an amplicon encompassing at least 5, 10, 15, 16, 17, 18, 19 or 20 nucleic acid residues upstream of the location of the indel and/or at least 5, 10, 15, 16, 17, 18, 19 or 20 nucleic acid residues downstream of the indel. In still another embodiment, the primers can be designed to generate an amplicon encompassing at least 5, 10, 15, 16, 17, 18, 19 or 20 nucleic acid residues upstream of the location of the indel and at least 5, 10, 15, 16, 17, 18, 19 or 20 nucleic acid residues downstream of the indel. In yet another embodiment, the primers can be designed to generate an amplicon encompassing 20 nucleic acid residues upstream of the location of the indel and 20 nucleic acid residues downstream of the indel. In such embodiments, in the nematodes having the indel, the primers will generate an amplicon of 103 nucleic acid bases and in the nematodes lacking the indel, the primers will generate an amplicon of 40 nucleic acid bases. In still another embodiment, the primers can be designed to generate an amplicon (lacking the indel) of at least 40, 50, 60, 70, 80, 90, 100 or 150 nucleic acid bases and/or at most 500, 600, 700, 800, 900 or 1 000 nucleic acid bases. In another embodiment, the primers can be designed to generate an amplicon (having the indel) of at least 103, 113, 123, 133, 143, 153, 163 or 213 nucleic acid bases and/or at most 563, 663, 763, 863, 963 or 1 063 nucleic acid bases.

In another example, the isolated genomic DNA (or a synthetic copy thereof) can be combined and contacted with at least one primer specific for the region (either upstream or downstream) where the indel can be putatively located in the Hco-acr-8 gene (or gene ortholog) and another primer specific for the indel itself under conditions to form a complex between the genomic DNA and the pair of primers (when the indel is present). In such embodiment, a complex will not be formed between the pair of primers and the genomic DNA of all nematodes tested since only the genomic DNA of the nematodes comprising the indel will specifically bind to both primers. A nucleic-acid polymerization step is then conducted and the resulting amplification products are characterized to determine the presence or absence of the indel. The characterization can be made based on the presence or absence of amplification products. If an amplification product is obtained, then it is considered that the indel is present (because both primers would have formed a complex with the genomic DNA). If no amplification products are obtained, then it is considered that the indel is absent (because only one primer (located outside the indel) would have formed a complex with the genomic DNA). In this example, a further confirmatory step which can include sequencing the amplification product or using a probe specific for the indel can also be used to characterize the amplification products (and confirm the presence of the indel).

In the embodiment in which primers are used to amplify the indel (or lack thereof) and only one of the primer is located either upstream or downstream of the position of the indel, the primers are designed in order to preferably locate in a region (e.g., between 20 and 25 nucleic acid base-long) of high identity between the different nematode strains. Further, in an embodiment, the primers can be designed to generate an amplicon encompassing at least 5, 10, 15, 16, 17, 18, 19 or 20 nucleic acid residues upstream of the location of the indel or at least 5, 10, 15, 16, 17, 18, 19 or 20 nucleic acid residues downstream of the indel. In yet another embodiment, the primers can be designed to generate an amplicon encompassing 20 nucleic acid residues upstream of the location of the indel or 20 nucleic acid residues downstream of the indel. In such embodiments, in the nematodes having the indel, the primers can generate an amplicon of at most 83 nucleic acid bases and in the nematodes lacking the indel, no amplicon will be generated. In still another embodiment, the primers can be designed to generate an amplicon (having the indel) of at least 40, 50, 60, 70, 80, 90, 100 or 150 nucleic acid bases and/or at most 500, 600, 700, 800, 900 or 1 000 nucleic acid bases.

In another example, the isolated genomic DNA (or a synthetic copy thereof) can be combined and contacted with a pair of primers both specific for the indel located in the Hco-acr-8 gene (or its corresponding gene ortholog). In such embodiment, a complex will not be formed between the pair of primers and the genomic DNA of all nematodes tested since only the genomic DNA of the nematodes comprising the indel will specifically bind to both primers. A nucleic-acid polymerization step is then conducted and the resulting amplification products are characterized to determine the presence or absence of the indel. The characterization can be made based on the presence or absence of amplification products. If an amplification product is obtained, then it is considered that the indel is present. If no amplification products are obtained, then it is considered that the indel is absent. In this example, a further confirmatory step which can includes sequencing the amplification product or using a probe specific for the indel can also be used to characterize the amplification products (and confirm the presence of the indel).

In the embodiment in which primers are used to amplify the indel (or lack thereof) and they are both located inside the indel, the primers are designed in order to preferably locate in a region (e.g., between 20 and 25 nucleic acid base-long) of high identity between the different nematode strains. Further, in an embodiment, the primers can be designed to generate an amplicon (having the indel) of at least 40, 50, 60 or 63 nucleic acid bases and/or at most 63, 60, 50 or 40 nucleic acid bases.

In still another example, the isolated genomic DNA (or a synthetic copy thereof) can be combined and contacted with at least three primers, a first one specific for a region upstream of the indel, a second specific for a region downstream of the indel and a third one specific for the indel itself and designed to provide an amplicon with the first or second primer. In such embodiment, a complex will be formed between the first primer, the second primer and the genomic DNA of all nematodes, irrespective of the presence of the indel. However, a complex will be formed between the first primer, the second primer, the third primer and the genomic DNA only in nematodes having the indel. A nucleic-acid polymerization step is then conducted and the resulting amplification products are characterized to determine the presence or absence of the indel. The characterization can be made based on the presence or absence of amplification products. The amplification products of an imidazothiazole anti-helmintic-sensitive nematodes will comprise two distinct amplification products (one resulting from the amplification between the first and the second primer and another one resulting from the amplification between the third and first or second primer) indicative of the presence of the indel. The amplification products of an imidazothiazole anti-helmintic-resistant nematodes will comprise a single amplification product (resulting from the amplification between the first and the second primer). The amplification products can also be characterized via their presence, their sequence or their size to determine if the indel is present. In this example, a further confirmatory step using a probe specific for the indel can also be used to characterize the amplification products (and confirm the presence of the indel).

In the embodiment in which three primers are used to amplify the indel (or lack thereof), the first, second and third primers are designed in order to preferably locate in a region (e.g., between 20 and 25 nucleic acid base-long) of high identity between the different nematode strains. Further, in an embodiment, the first and second primers can be designed to generate an amplicon encompassing at least 5, 10, 15, 16, 17, 18, 19 or 20 nucleic acid residues upstream of the location of the indel and/or at least 5, 10, 15, 16, 17, 18, 19 or 20 nucleic acid residues downstream of the indel. In still another embodiment, the first and second primers can be designed to generate an amplicon encompassing at least 5, 10, 15, 16, 17, 18, 19 or 20 nucleic acid residues upstream of the location of the indel and at least 5, 10, 15, 16, 17, 18, 19 or 20 nucleic acid residues downstream of the indel. In yet another embodiment, the first and second primers can be designed to generate an amplicon encompassing 20 nucleic acid residues upstream of the location of the indel and 20 nucleic acid residues downstream of the indel. In such embodiments, in the nematodes having the indel, the first and second primers will generate a first amplicon of 103 nucleic acid bases and in the nematodes lacking the indel, the primers will generate a first amplicon of 40 nucleic acid bases. In still another embodiment, the first and second primers can be designed to generate a first amplicon (lacking the indel) of at least 40, 50, 60, 70, 80, 90, 100 or 150 nucleic acid bases and/or at most 500, 600, 700, 800, 900 or 1 000 nucleic acid bases. In another embodiment, the first and second primers can be designed to generate an amplicon (having the indel) of at least 103, 113, 123, 133, 143, 153, 163 or 213 nucleic acid bases and/or at most 563, 663, 763, 863, 963 or 1 063 nucleic acid bases.

In such embodiment, the first or second primer and the third primer can be designed to generate an amplicon encompassing at least 5, 10, 15, 16, 17, 18, 19 or 20 nucleic acid residues upstream of the location of the indel or at least 5, 10, 15, 16, 17, 18, 19 or 20 nucleic acid residues downstream of the indel. In yet another embodiment, the first or second primer and the third primer can be designed to generate an amplicon encompassing 20 nucleic acid residues upstream of the location of the indel or 20 nucleic acid residues downstream of the indel. In such embodiments, in the nematodes having the indel, the first or second primer and the third primer can generate a second amplicon of at most 83 nucleic acid bases and in the nematodes lacking the indel, no amplicon will be generated. In still another embodiment, the first or second primer and the third primer can be designed to generate an amplicon (having the indel) of at least 40, 50, 60, 70, 80, 90, 100 or 150 nucleic acid bases and/or at most 500, 600, 700, 800, 900 or 1 000 nucleic acid bases.

As used herein, a primer is an oligonucleotide used to initiate DNA replication. Typically, a primer is a short oligonucleotide that may be about 10, about 15, about 20, about 25, about 30, about 35, about 40, about 45, about 50, about 55, about 60, about 65, about 70, about 75, about 80, about 85, about 90, about 95, about 100 or more nucleotides. The primer-driven amplification is observed in PCR, allele-specific RT-PCR and qRT-PCR methods. In some embodiment, the primer is identical to the complementary strand of the target nucleic acid sequence intended to be amplified (or with which the primer is intended to form a complex).

The amplification products or amplicons can be used, for subsequent analysis, such as by Southern blot, sequencing or SSCP.

In an embodiment, a probe can be used for determining of the presence of the absence of the indel in the Hco-acr-8 gene (or its corresponding gene ortholog). The probe can be designed to specifically bind to a Hco-acr-8 gene (or its corresponding gene ortholog) lacking the indel (for example, the probe can be designed to encompass the upstream and downstream regions (e.g., 20, 30, 40 or 50 nucleic acid bases upstream or downstream of the indel) where the indel between exon 2 and exon 3 can be present). Alternatively, the probe can be designed to specifically bind to a Hco-acr-8 gene (or its corresponding gene ortholog) having the indel (for example, the probe can be designed to encompass a region spanning both the Hco-acr-8 gene and the indel). The probe and the genomic DNA of the nematode that is being screened are placed into conditions favoring specific interactions (binding) between the probe and the genomic DNA. In the methods described herein, the formation (or lack of formation) of a complex between the probe and the genomic DNA of the nematode is thus indicative of the phenotype of the nematode. For example, when the probe is specific for the presence of the indel, the formation of a complex between the probe and the screened genomic DNA indicates that the nematode is likely susceptible to an imidazothiazole anti-helmintic whereas the lack of formation of the complex indicates that the nematode is likely resistant to an imidazothiazole anti-helmintic. In another example, when the probe is specific for the absence of the indel, the formation of a complex between the probe and the screened genomic DNA indicates that the nematode is likely resistant to an imidazothiazole anti-helmintic whereas the lack of formation of the complex indicates that the nematode is likely susceptible to an imidazothiazole anti-helmintic.

As used in the context of the present disclosure, a “probe” may be one or more molecules that are capable of binding to, or associating with, the genomic nucleotide sample to determine the presence or absence of the indel in the Hco-acr-8 gene (or its corresponding gene ortholog). The probe may be, for example, an oligonucleotide, an aptamer or an antibody.

An “oligonucleotide” may comprise any size, shape and composition that is suitable for use in the context of the present disclosure. Preferably, an oligonucleotide of the disclosure may comprise DNA, RNA, synthetic nucleotides, non-natural nucleotides, altered nucleotides, or combinations of one or more thereof. The term “oligonucleotide” refers to naturally-occurring species or synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art. In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the disclosure. Oligonucleotides may also include species that include at least some modified base forms. Thus, purines and pyrimidines other than those normally found in nature may be so employed. Similarly, modifications on the furanosyl portions of the nucleotide subunits may also be affected, as long as the essential tenets of this disclosure are adhered to. Examples of such modifications are 2′-O-alkyl- and 2′-halogen-substituted nucleotides. Some non-limiting examples of modifications at the 2′ position of sugar moieties which are useful in the present disclosure include OH, SH, SCH₃, F, OCH₃, OCN, O(CH₂), NH₂ and O(CH₂)nCH₃, where n is from 1 to about 10. Such oligonucleotides are functionally interchangeable with natural oligonucleotides or synthesized oligonucleotides, which have one or more differences from the natural structure. All such analogs are comprehended by this disclosure so long as they function effectively to specifically hybridize with the Hco-acr-8 gene (or gene ortholog) to detect the presence or absence of the indel. An oligonucleotide may be of any length that is suitable for use in methods of the disclosure. In embodiments of the disclosure, an oligonucleotide may comprise a sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, or more nucleotides.

As used herein, an “aptamer” may be a nucleic acid or a peptide molecule that binds specifically to the Hco-acr-8 gene (or its corresponding gene ortholog) and allows for the detection of the presence or absence of the indel. For example, in solution, a chain of nucleotides may form intramolecular interactions that fold the aptamer into a complex three-dimensional shape. The shape of that aptamer allows it to bind tightly against the surface of its target molecule. Because of the diversity of molecular shapes that exists for nucleotide and amino acid sequences, aptamers may be obtained for a wide array of molecular targets, including, but not limited to, nucleic acid molecules, enzymes, membrane proteins, viral proteins, cytokines, growth factors, and immunoglobulins.

An aptamer may be a nucleic acid molecule. Said aptamer may comprise DNA, RNA, synthetic nucleotides, non-natural nucleotides, altered nucleotides, or combinations of one or more thereof. The nucleic acid aptamer may be single-stranded or double-stranded. A nucleic acid aptamer may comprise a sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, at least 300, at least 350, at least 400, at least 500, or more nucleotides. A preferred nucleic acid aptamer may be a single stranded nucleic acid molecule and comprise a sequence of less than about 100 nucleotides. Alternatively, an aptamer may be a peptide molecule. A peptide aptamer may comprise a sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200 or more amino acid residues. A preferred peptide aptamer may comprise a sequence of between about 15 to about 75 amino acid residues.

A probe of the disclosure may be prepared according to standard techniques known to a skilled person. For example, a probe may be produced synthetically, recombinantly or may be isolated from a natural source. In one embodiment, the source may be a biological source, for example, from a microorganism (e.g. a bacteria, a yeast or a virus), an animal (e.g. a mouse, a rat, a rabbit, a goat, or a human as well as cells or cell lines derived therefrom), or a plant (as well as cells or cell lines derived therefrom).

In the context of the disclosure, a probe may mean one probe or more than one probe. In one embodiment, a single probe may be used to detect the presence or absence of the indel. A skilled person would appreciate that one or more probes may be useful in the context of the disclosure and may depend on the genotyping approach taken.

Probe design and production are known in the art. Generally, a probe may be produced recombinantly, synthetically, or isolated from a natural source, e.g. from a cell, an animal or a plant. However, a skilled person would appreciate that probe production may depend on the type of probe at issue.

In yet another embodiment, it is possible to determine the sequence of the genomic DNA of the nematode to ascertain the presence or absence of the indel. Such sequencing methods are well know in the art and can be preceded by an optional amplification step.

### Tools and Commercial Packages for Detecting an Imidazothiazole Anti-Helmintic Resistance

The present disclosure also provides an isolated nucleic acid molecule having or consisting essentially of the nucleic acid sequence of SEQ ID NO: 9. In embodiments of the disclosure, the nucleic acid molecule is at least about 80%, at least about 81%, at least about 82%, at least about 83%, at least about 84%, at least about 85%, at least about 86%, at least about 87%, at least about 88%, at least about 89%, at least about 90%, at least about 91%, at least about 91%, at least about 92%, at least about 93%, at least about 94%, at least about 95%, at least about 96%, at least about 97%, at least about 98%, at least about 99% or 100% identical to SEQ ID NO: 9. Such nucleic acid molecule (or fragment thereof) can be successfully used as a probe for the detection of the indel or as a primer for the amplification of the indel (or a part thereof).

In the context of the present disclosure, the probe is at least 30 base pairs, 35 base pairs, 40 base pairs, 45 base pairs, 50 base pairs, 55 base pairs, 56 base pairs, 57 base pairs, 58 base pairs, 59 base pairs, 60 base pairs, 61 base pairs, 62 base pairs or 63 base pairs. In another embodiment, its nucleotide sequence is substantially identical (at least 80%, 85%, 90%, 91%, 92%, 93%, 94%, 95%, 96%, 97%, 98%, 995 or 100% identity over the length of the probe) to the nucleotide sequence of the indel. In still another embodiment, the probe can be derived directly from the nucleotide sequence of the indel. Because the probe is designed to detect the presence of the indel, it is designed to bind to the indel sequence and be detected when complexed with the indel. Further, because the indel could be present in other genomic location than the Hco-acr-8 gene, it is contemplated that the probe also comprises nucleotides specific for the region neighboring the indel in the Hco-acr-8 gene (or its corresponding ortholog). However, in such embodiment, the probe must be designed not to bind to the genomic DNA which does not bear the indel.

The probe of the disclosure can be a TaqMan probe. TaqMan™ probes are usually designed to be located near one of the PCR primers so that when the Taq polymerase reaches the probe, it will cleave its 5′ end. This will separate the fluorophore attached to the 5′ end of the probe and notably from a strong quencher attached to the 3′ end that would normally absorb all the light emitted from the fluorophore. Alternatively, the probe of the disclosure can be a Molecular Beacons™ probe. Molecular Beacons™ probes, on the other hand, have complementary sequences at the ends, often referred to as “panhandles”, which when hybridized together bring a fluorophore at the 5′ end in very close proximity to a quencher at the 3′ end. Measurable light is emitted from the fluorophore only when the probe hybridizes to its target and the 5′ and 3′ ends are spatially separated. Molecular Beacons™ probes are designed so that their complementary ends reanneal near the annealing temperature of the PCR reaction. In another embodiment, the probe of the disclosure can be a FRET probe. FRET probes involve transfer of light not to a quencher but rather to a second fluorophore that is not excited by the wave length used by the analyzer which excites only the fluorophore fluorescein. The second fluorophore then absorbs the light emitted by the fluorescein moiety to emit at a longer wave length. This occurs when two probes hybridize to adjoining sequences on the target such that the 3′ end of one is very near the 5′ end of the other.

In the context of the present disclosure, the primer is at least 10 base pairs, 11 base pairs, 12 base pairs, 13 base pairs, 14 base pairs, 15 base pairs, 16 base pairs, 17 base pairs, 18 base pairs, 19 base pairs, 20 base pairs, 21 base pairs, 22 base pairs, 23 base pairs, 24 base pairs or 25 base pairs. In another embodiment, its nucleotide sequence is substantially identical (at least 80%, 85%, 90%, 91%, 92%, 93%, 94%, 95%, 96%, 97%, 98%, 995 or 100% identity over the length of the primer) to the nucleotide sequence of its target (the indel or neighboring regions). In still another embodiment, the probe can be derived directly from the nucleotide sequence of the indel or the neighboring regions of the Hco-acr-8 gene (or its corresponding ortholog).

In order to conduct the methods presented herewith, commercial packages are also provided. The commercial packages comprise means for determining the presence or absence of an indel having the sequence of SEQ ID NO: 9 and being located in the a Hco-acr-8 gene or a Hco-acr-8 gene ortholog. The commercial package can also comprise instructions for characterizing the nematode based on the presence or absence of the indel. Such instructions may comprise the information conveyed in the “characterizing” step of the methods described above. In an embodiment, means for detecting the indel comprises a probe (or a plurality of probes) for determining the presence or absence of the indel. As indicated above, the probes can be specific for the indel or for the corresponding region not encompassing the indel. In yet another embodiment, means for detecting the indel comprises at least one pair of primers for determining the presence of absence of the indel. As indicated above, the primer(s) can be specific for the indel or the region surrounding the indel.

The present invention will be more readily understood by referring to the following examples which are given to illustrate the invention rather than to limit its scope.

### Example

### H. contortus Isolates

Levamisole susceptible and resistant isolates of H. contortus were obtained from different locations. Information on the different parasitic nematode isolates investigated is detailed below.

Levamisole Susceptible Isolates.

PF23 (PF) is a laboratory isolate of H. contortus never exposed to drug treatment (Rajan et al., 2002). Kindly supplied by Fort Dodge Animal Health, Princeton, N.J., USA. ISE was kindly provided by Dr Claude Charvet, INRA Tours, France. This isolate is susceptible to all main classes of anthelmintic, including levamisole (Otsen et al., 2001; Roos et al., 2004). The ISE isolate has been sequenced for the H. contortus genome sequencing project. Zaire was kindly provided by Dr Claude Charvet, INRA Tours, France. This isolate was collected in the Ituri region (now Congo) and is levamisole susceptible. No levamisole treatment had been performed for 20 years at the time the Zaire isolate was collected and anthelmintic treatments had been very rare. It was then maintained under experimental conditions for 10 years without any treatment. Zaire has been shown not to produce the truncated form Hco-ACR-8b. Courtion was kindly provided by Dr Ronald Kaminsky, Novartis Animal Health, Switzerland. Levamisole efficacy was found to be 100% against this isolate. Courtion was isolated in Switzerland in 2004. Roggliswil was kindly provided by Dr Ronald Kaminsky, Novartis Animal Health, Switzerland. Levamisole efficacy was found to be >99% against this isolate. Roggliswil was isolated in Switzerland in 2011. CRA was kindly provided by Dr Ronald Kaminsky, Novartis Animal Health, Switzerland. Overall, this isolate is susceptible to levamisole. CRA was isolated in the Republic of South Africa in 1984.

Levamisole Resistant Isolates.

Kokstad was kindly provided by Dr Claude Charvet, INRA Tours, France. This isolate is resistant to levamisole and expressed the truncated form Hco-ACR-8b (Fauvin et al., 2010). The origin of this isolate is South Africa. Cedara was kindly provided by Dr Claude Charvet, INRA Tours, France. This isolate from South Africa is resistant to levamisole and expressed the truncated form Hco-ACR-8b (Fauvin et al., 2010). RHS6 was kindly provided by Dr Claude Charvet, INRA Tours, France. This isolate is resistant to levamisole and expressed the truncated form Hco-ACR8-b (Hoekstra et al., 1997; Fauvin et al., 2010). RHS6 before being selected for levamisole resistance was collected in Zimbabwe. UGA/2004 was kindly provided by Dr. Adrian J. Wolstenholme, University of Georgia, United States of America. The UGA/2004 isolate of H. contortus was originally obtained from sheep at the University of Georgia (UGA) Sheep Unit in 2004. Clinical evidence suggested H. contortus were resistant to multiple anthelmintic classes included levamisole. In addition, the truncated isoform Hco-ACR8-b was found in this isolate (Williamson et al., 2011). Howick was kindly provided by Dr Ronald Kaminsky, Novartis Animal Health, Switzerland and is resistant to levamisole (van Wyk et al., 1997).

DNA Extractions.

Total genomic DNA was extracted with an extraction kit (DNeasy® blood and tissue kit 250, Cat No. 69506 Qiagen Inc, Mississauga, Ontario, Canada) for adult worms from isolates CRA, PF, ISE, Zaire, Kokstad, Cedara, RHS6 and Howick. Pools of 50 larvae from isolates UGA/2004, Courtion and Roggliswil were collected under the microscope (magnification ×50) with a 10 μl Eppendorf pipette for each isolate. A volume of 15 μl of lysis buffer (50 mM KCl, 10 mM Tris, and 2.5 mM MgSO₄) with 1.5 μl of proteinase K and 1.5 μl of β-mercapto-ethanol were added to the larvae in a PCR tube. The DNA of the 50 larvae was extracted during 2 hours at 60° C. with an additional 15 minutes at 94° C. to inactivate the proteinase K.

DNA Amplification and Sequencing.

A total of 80 single adult males from Kokstad, Cedara, RHS6, ISE, Zaire, PF, CRA and Howick and pools of 50 larvae from UGA/2004, Courtion and Roggliswil were used for PCR and sequencing of each area of interest. Amplification of the Hco-acr-8 gene by PCR from genomic DNA was achieved using primers listed in Table 1. One μl of DNA template was amplified in a mix containing 5 U Platinum Taq High fidelity polymerase (Invitrogen #11304029, Invitrogen Canada Inc., Burlington, Ontario, Canada), 3 mM MgSO₄ (Invitrogen #11304029), 40 nM dNTPs (Invitrogen #10297018) and 0.2 mM of each forward and reverse primer. Amplification was performed with a program of 3 min at 94° C. followed by 40 cycles at 94° C. for 1 min, 60° C. for 1 min and 68° C. for 1 min and a final elongation step at 68° C. for 10 min. A negative control was run for each PCR performed during this study. PCR contamination in the negative controls was checked by electrophoresis on agarose gel. DNA amplicons, in the absence of contamination in the negative control, were sequenced at the Genome Quebec Innovation Centre, Montreal. Forward and reverse sequences were aligned to create a consensus for each individual and each pool. Multiple alignments among consensus sequences were performed with Genious software version 5.6 (Biomatters Ltd) (Drummond et al., 2012).

TABLE 1
Forward and reverse primer sequences
for each region of interest.

| Regions | | |
|---|---|---|
| of | Primer | Alternative |
| interest | sequences | primer sequences |
| A | For: | For: |
| | AGTGGAATCGAATC | CGTCGTATACATAGT |
| | GTTATGCTGAACA | GGAATC |
| | (SEQ ID NO: 1) | (SED ID NO : 3) |
| | Rev: GGACAGAGA | Rev: YMYMTTATAT |
| | CACAAAATTGGGG | CCCGARAARA |
| | (SEQ ID NO: 2) | (SEQ ID NO: 4) |
| B | For: TTCCATACC | |
| | CATTGACCATC | |
| | (SEQ ID NO: 5) | |
| | Rev: AAAGGCGAA | |
| | GAAGTACAAGG | |
| | (SEQ ID NO: 6) | |
| C | For: CGTCGCAAC | |
| | GAAAGAGWCG | |
| | (SEQ ID NO: 7) | |
| | Rev: CCCACGCTA | |
| | ATTTATAGTCG | |
| | (SEQ ID NO: 8) | |

Truncated Isoform Hco-Acr-8b Identification.

The presence of the truncated isoform Hco-acr-8b was carried out for the isolates ISE, Courtion, Roggliswil, PF, CRA and Kokstad. RNA extraction was performed with Trizol® (Invitrogen-15596-026) from a pool of 10,000 larvae from CRA, Courtion, Roggliswil and 10 adults from PF and Kokstad. cDNA synthesis and amplification of transcripts of β-tubulin and Hco-acr-8b (HAX) protocols are presented in Fauvin et al., 2010. Kokstad was used as a positive control for amplification of Hco-acr-8b isoform after RT-PCR. Amplification of the β-tubulin isotype 1 transcript was carried out for all of the isolates to confirm cDNA amplification success.

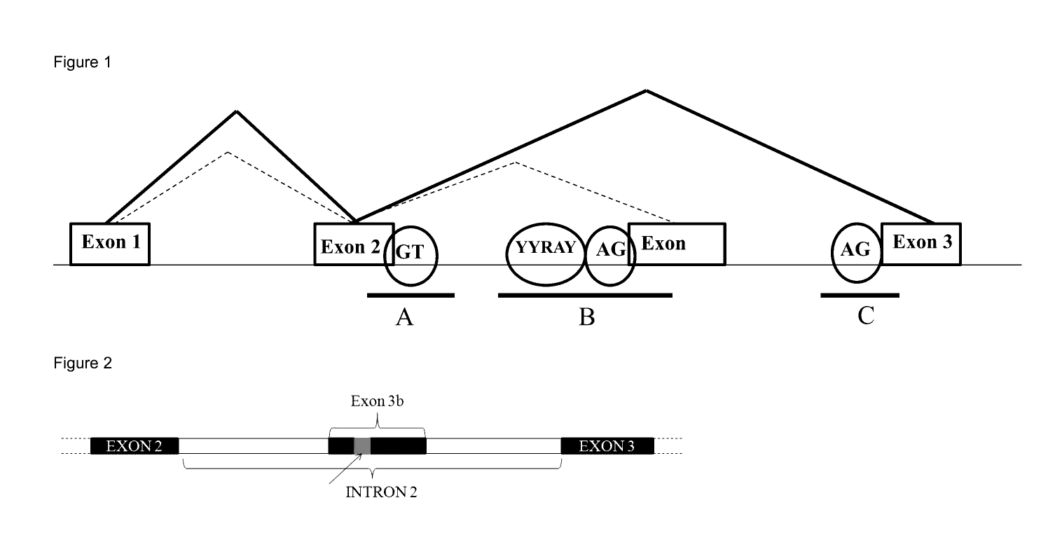

FIG. 1 shows the alternate splice variants and the truncated Hco-acr-8b positions. The splice site donor GT and acceptor AG are highlighted as well as the putative splicing branch point sequence (YYRAY).

The approach of sequencing PCR products from diploid individuals is rapid and allowed a survey of a large number of individual worms. Failure of PCR primers to amplify, due to underlying polymorphism, is always a concern for a survey of this type. In this example, several independent sets of PCR primers were used with sequence derived from successful amplification in each case. For sequence region B, 100% of the 64 individuals produced an amplicon with at least one set of primers. Region A (54 individuals) and C (48 individuals) were successful for 71% and 77% of individuals, respectively. For the remaining 29% and 23%, the failure can be attributed to the primers, since the template DNA was successfully amplified for region B in each case. In the three regions, it was counted 360 SNP sites with region A (720 bp) harboring 150 SNPs, region B (680 bp) with 67 SNPs, and region C (620 bp) with 143 SNPs.

The presence of 13 insertion-deletion (indel) variants of 4 bp to 63 bp meant that individuals heterozygous for the presence of an indel produced sequence that was unreadable beyond the point of the indel. This was observed in 7, 9 and 6% of individuals from regions A, B and C where the remaining region of readable sequence was used to assess SNP variation. The use of diploid sequencing also meant that individuals heterozygous for more than one position can only be used to infer the underlying haplotypes if assumptions about linkage and Hardy-Weinberg equilibrium are made. The individuals studied came from a range of geographically separate locations, which together with the presence of alleles that do not amplify means that such assumptions would be invalid. Homozygous male individuals were used for alignment and substitution analysis.

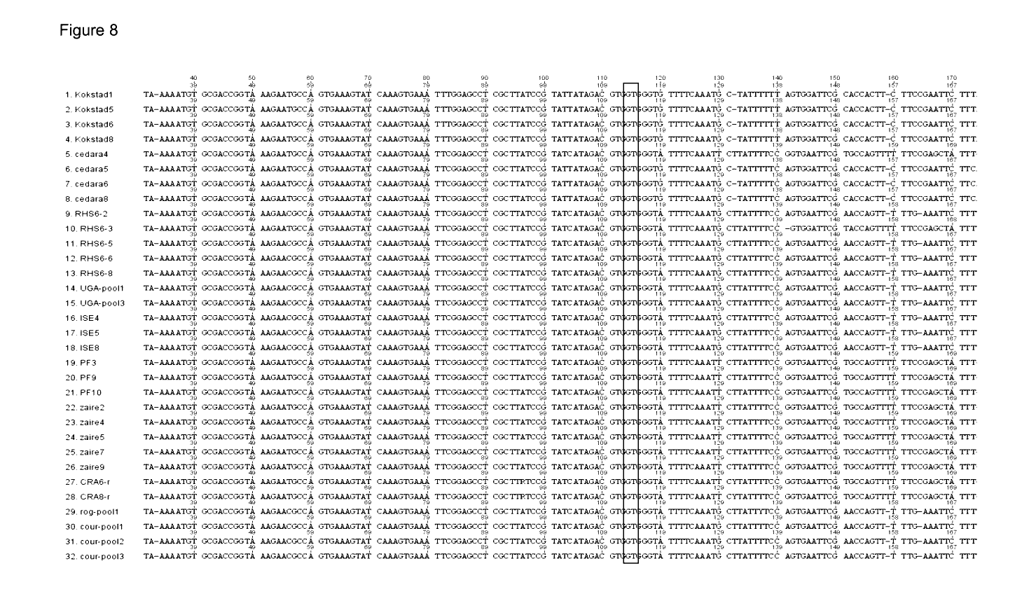

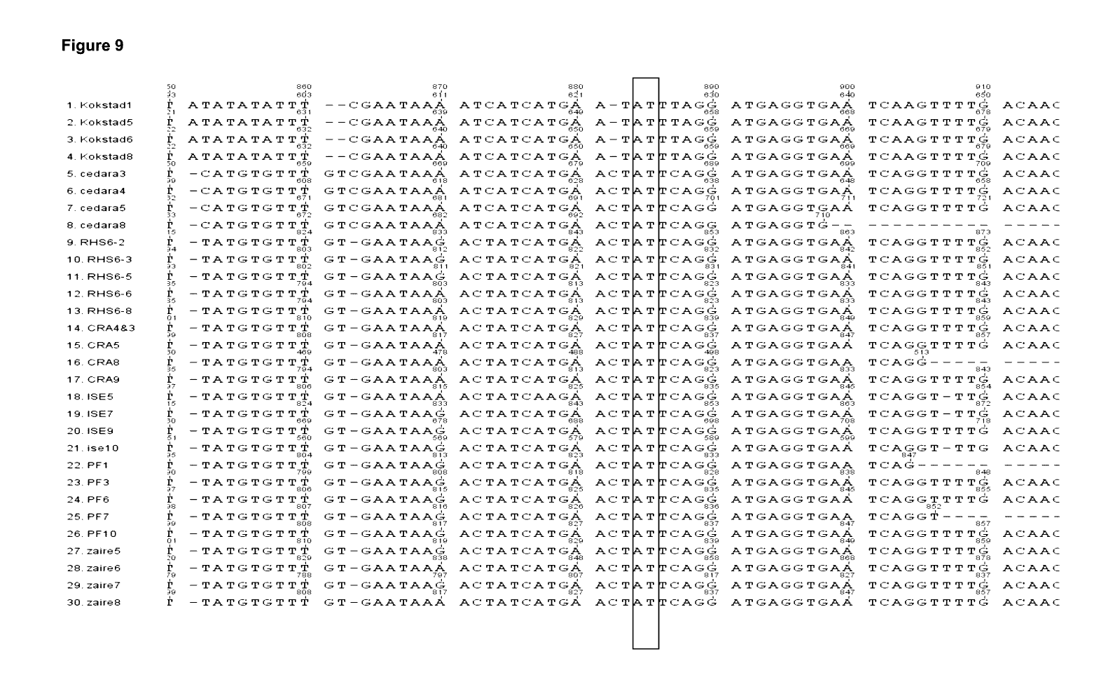

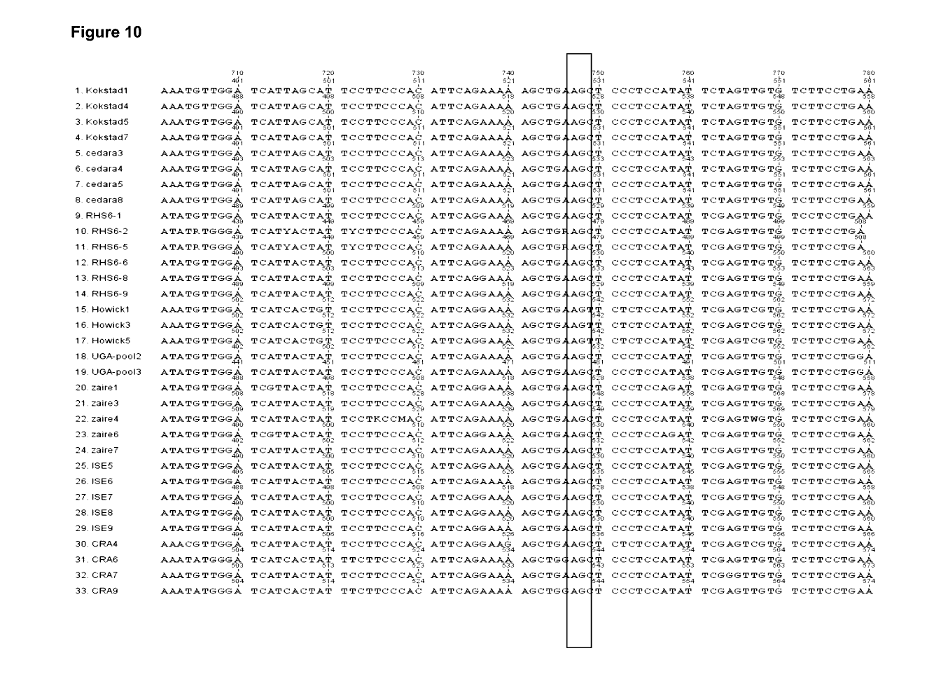

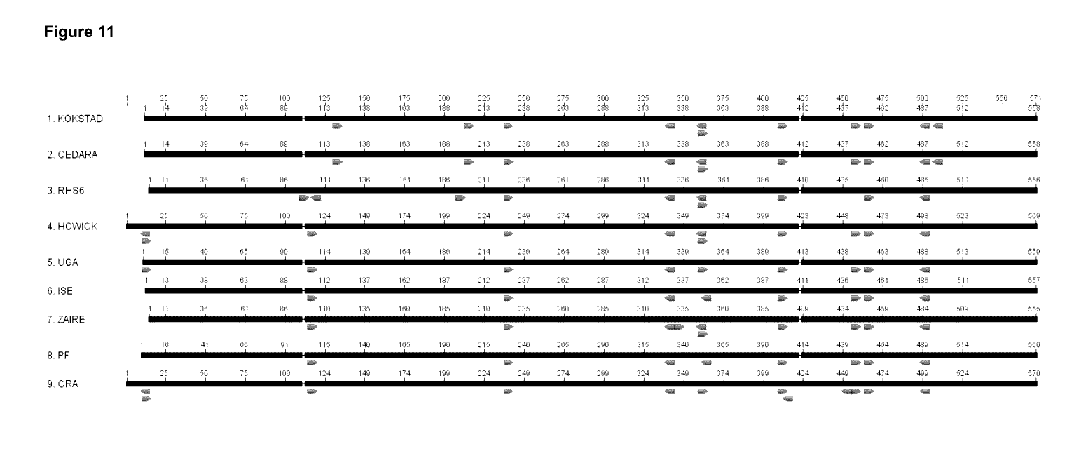

PCR products from individual male worms were sequenced in order to examine sequence variation of the Hco-acr-8 gene at the three regions of interest: the splice donor site downstream of exon 2, the splice branch point and acceptor site upstream of exon 3b and the splice acceptor site upstream of exon 3 regions A, B and C (FIG. 1). All sequences had the GT donor splice site at the start of intron 2 (FIG. 8), the AG splice acceptor site (FIG. 9) before the start of exon 3b and the AG splice acceptor site (FIG. 10) at the end of intron 2. All sequences had at least one branch point sequence, YYRAY, within 200 bp from the AG acceptor splice site at the end of intron 2 (FIG. 11). Based on this data, there was no reason to believe that any change at these positions was responsible for creating the truncated transcript.

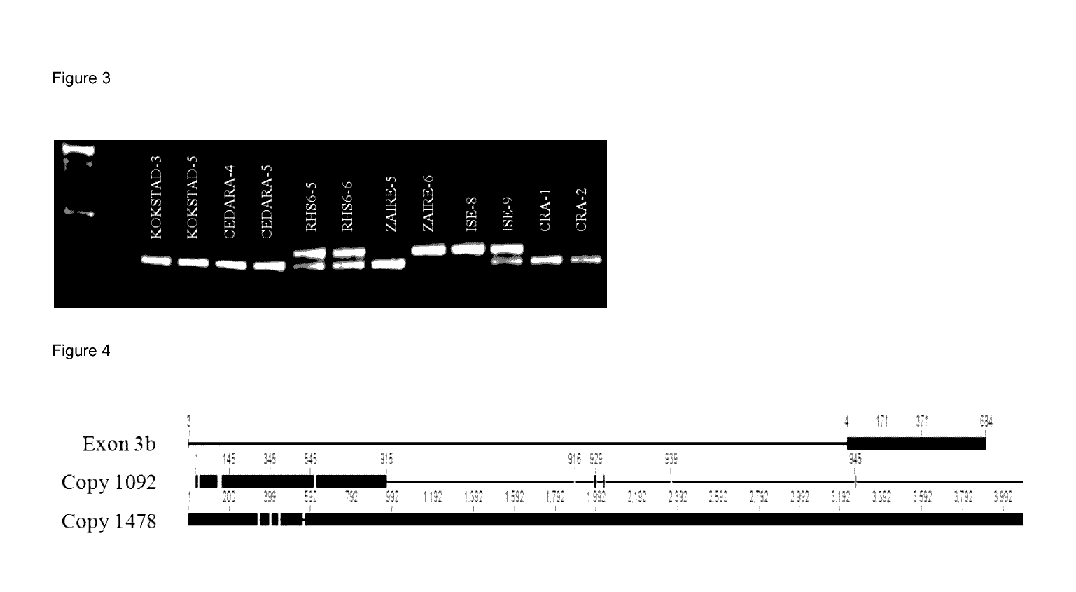

During the analysis of sequences of the AG splice variant site upstream of the exon 3b, the presence of an indel of 63 bp was detected (FIG. 2). This indel sequence was similar in each sample, in which it was present, and causes the amplicons to have different sizes that are visible on agarose gel electrophoresis (FIG. 3). The nucleotide sequence and the amino acid sequences of the indel are shown in Table 2. A Blast search of version 1 of the H. contortus genome identified 220 copies of the sequence present within a variety of other unique gene sequences. A phylogenetic tree of the region B haplotype sequence rooted at the midpoint suggested the 63 bp indel is an insertion of a mildly repetitive sequence into the Hco-acr-8 gene (data not shown). We found no strong evidence that this sequence is present in different Caenorhabditis species, Ascaris, Pristionchus, Trichuris or any other non-trichostrongylid organism; this repetitive sequence seems to be specific to trichostrongylid parasites. When comparing the occurrence of the indel with the levamisole resistance phenotype of individuals from Kokstad, Cedara, ISE, Zaire, Howick, CRA, UGA/2004, PF, Courtion, Roggliswil and RHS6, a significant difference (p<0.01) between levamisole resistant (33 individuals and 2 pools) and susceptible (31 individuals and 7 pools) phenotype was found. From our data, the addition of the 63 bp occurs more often in susceptible isolates compared with resistant isolates.

TABLE 2
Indel genomic DNA sequence (63 bp);
this sequence was constant among all
the sequences analyzed which had the
indel in exon 3b.

| | |
|---|---|
| Indel | TTTTGACTTGATGTTTTGTTAACTGCTGT |
| Sequence | TATATCGCCGCAGTACGCGTAAGGCTGAT |
| Nucleotides | TACTG (SEQ ID NO: 9) |
| Frame 1 | F(STOP)LDVLLTAV |
| | ISPQYA(STOP)G(STOP)L |
| | L (SEQ ID NO: 10) |
| Frame 2 | FDLMFC(STOP)LLL |
| | YRRSTRKADY |
| | (SEQ ID NO: 11) |
| Frame 3 | LT(STOP)CFVNCCY |
| | IAAVRVRLIT |
| | (SEQ ID NO: 12) |
| Reverse | Q(STOP)SALRVLRR |
| Frame 1 | YNSS(STOP)QNIKS |
| | K (SEQ ID NO: 13) |
| Reverse | SNQPYAYCGD |
| Frame 2 | ITAVNKTSSQ |
| | (SEQ ID NO: 14) |
| Reverse | VISLTRTAAI |
| Frame 3 | (STOP)QQLTKHQVK |
| | (SEQ ID NO: 15) |

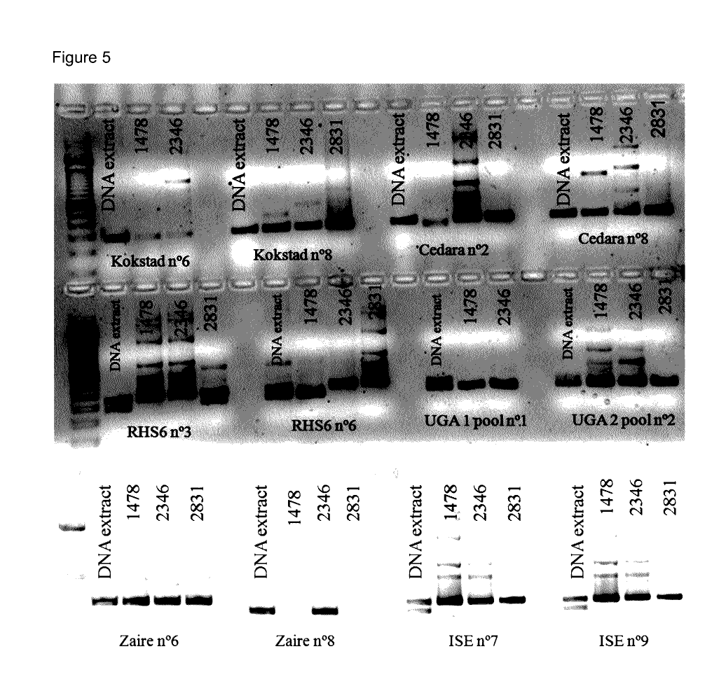

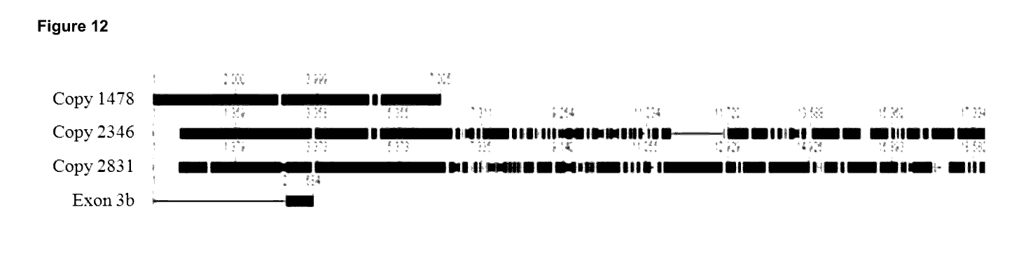

The H. contortus genome data version 1 contains 4 different scaffolds homologous to Hco-acr-8 (scaffold2831.1_size35112, scaffold2346.1_size44412, scaffold1478.1_size70794_1-10639 and scaffold1092.1_size89268). Copy 1092 did not contain the region B as indicated in FIG. 4. The copies 1478, 2346 and 2831 are similar but diverge significantly beyond the first 7000 bp (FIG. 12). This could be the result of allelic variation or the presence of more than one genetic locus encoding Hco-acr-8. To resolve this, the region B amplicon where the polymorphism due to the presence of the 63 bp insertion is long enough to be visualized by electrophoresis and the primers had a 100% success rate was examined. Primers specific for each copy (1478, 2346 or 2831) were used to amplify DNA samples of individuals and 50 pooled larvae followed by a second PCR of these products using primers located around the indel in exon 3b. In parallel, another PCR was performed with the same set of primers to amplify the indel area using the DNA extract as template. FIG. 5 presents the results of these PCRs. The amplifications of RHS6-6, ISE7 and ISE9 presented two bands when the DNA extract is amplified directly and only one band after amplification of the copy 1478, 2346 or 2831 followed by a nested amplification with the primers for the region containing the indel. If the scaffolds represent multiple copies of Hco-acr-8 then each individual would produce at least two bands. With the exception of one individual (RHS6-3 with copy 2831), we observed only one band of either size confirming that different scaffolds do represent allelic variants of a single genetic locus.

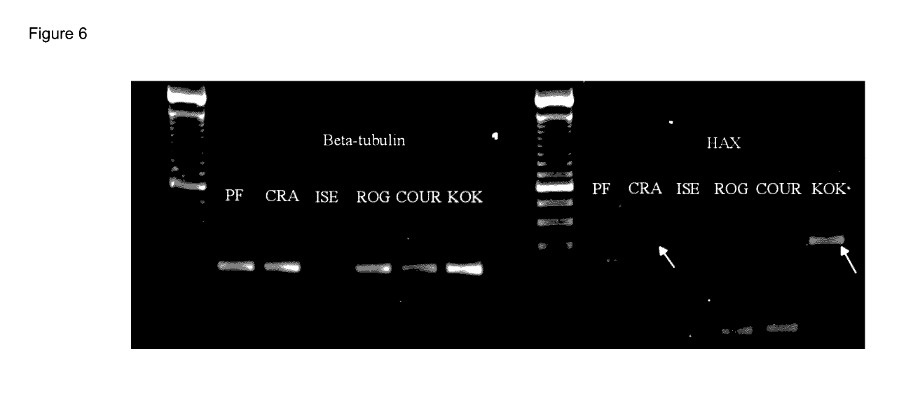

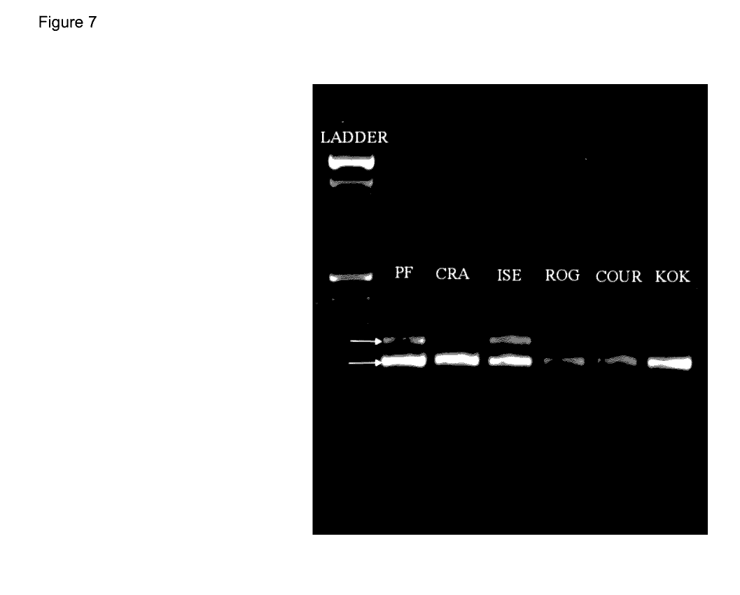

Reverse-Transcriptase PCR (RT-PCR) was performed with RNA from pools of larvae or adults and the presence of the insertion was identified in these isolates to create a relationship between genotype (genetic marker) and phenotype (presence of truncated transcript Hco-acr-8b). The isolate Kokstad was used as a positive control for the presence of the transcript Hco-acr-8b on the gel. The β-tubulin isotype 1 transcript was amplified and detected in each of the isolates to confirm proper RNA extraction and successful synthesis of the cDNA (Fauvin et al., 2010). With the same isolates, the presence of the transcript Hco-acr-8b (called HAX in Fauvin et al., 2010) and the presence of the indel were performed (FIGS. 6 and 7).

Detection of the truncated Hco-acr-8b transcript (mRNA) currently requires RNA as a template, which is technically demanding in a test as a marker for resistance and likely would preclude population studies on individual worms, larvae or eggs. Ideally, to detect the molecular change associated with the alternative splicing, leading to creation of the truncated transcript form, gDNA is preferable. This provided the rational for investigating polymorphism around the splice signals associated with the truncated Hco-acr-8b transcript and developing a novel test that can be used to detect levamisole resistance.

The first possibility could have been a mutation at the splice variant sites which would have created the truncated isoform to be translated. Such a mutation was previously found within intron 5 of the gene NF2 in humans which creates a branch point. This branch point leads to the activation of a cryptic exon involved in neurofibromatosis 2. In the nematode isolates screened, all the sequences possessed the AG and GT sites at the same positions and a branch point that would be needed for the production of the truncated Hco-acr-8b.

Fauvin and colleagues (2010) sequenced the transcript Hco-acr-8b (GU168769.1); the insertion is not present in this sequence. An addition of 63 bp within the sequence of exon 3b would create a different transcript and would not lead to the transcript Hco-acr-8b described in Fauvin et al. (2010) and Williamson et al. (2011). The different reading frames present a STOP codon in the sequence which could dramatically change the Hco-ACR-8 protein, and lead to a different phenotype of the parasite.

It was observed that the levamisole susceptible isolates ISE, Courtion and Roggliswil lack the transcript form (FIG. 6) and harbor the 63 bp insertion (FIG. 7). A relationship between the genotype (insertion of 63 bp) and the phenotype (absence of Hco-acr-8b transcript) was thus observed. The presence of the insertion of 63 bp, found within the sequence of exon 3b, was statistically linked to the phenotype of levamisole susceptibility and was not the result of gene duplication. Concerning the isolate CRA, overall this isolate is susceptible to levamisole but harbored the HAX transcript at a low level. Electrophoresis of the PCR amplicon showed predominantly, a band without the addition of 63 bp. The results with CRA suggest that while this isolate may be predominantly susceptible, there may be some individuals in the isolate which show characteristics associated with a predisposition to levamisole resistance.

There was a link between the insertion of 63 bp presented here and the expression of HAX in H. contortus isolates and evidence from previous studies (Fauvin et al., 2010; Williamson et al, 2011; Sarai et al., 2013: the present study) emphasized the link between the expression of HAX and levamisole resistance.

A genetic marker to detect levamisole resistance in the field would represent a real advance for livestock industries where levamisole resistance can lead to the failure of parasite control and economic losses. The deletion of the 63 bp indel was demonstrated to be statistically linked to the resistance phenotype. Based on our data, the insertion is significantly more frequent in susceptible individuals compared with resistant individuals.

Levamisole resistance has been related to the presence of a truncated transcript Hco-acr-8b in Haemonchus contortus isolates. The truncated isoform activation was investigated at DNA level in order to highlight the mechanisms causing this phenomenon to appear. The deletion of a 63 bp indel in the exon 3b sequence has been found to be correlated with the levamisole resistance phenotype.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Coles G C, Jackson F, Pomroy W E, Prichard R K, von Samson-Himmelstjerna G, Silvestre A, Taylor M A, Vercruysse J. The detection of anthelmintic resistance in nematodes of veterinary importance. Vet Parasitol. 2006 Mar. 31; 136(3-4):167-85. Epub 2006 Jan. 19.
Drummond, A. J., Ashton, B., Buxton, S., Cheung, M., Cooper, A., Duran, C., Field, M., Heled, J., Kearse, M., Markowitz, S., Moir, R., Stones-Havas, S., Sturrock, S., Thierer, T., Wilson, A., 2012. Geneious v 5.6. Available from http://www.geneious.com.
Fauvin, A., Charvet, C., Issouf, M., Cortet, J., Cabaret, J., Neveu, C., 2010. cDNA-AFLP analysis in levamisole-resistant Haemonchus contortus reveals alternative splicing in a nicotinic acetylcholine receptor subunit. Mol. Biochem. Parasitol. 170, 105-107.
Hoekstra, R., Borgsteede, F. H., Boersema, J. H., Roos, M. H., 1997. Selection for high levamisole resistance in Haemonchus contortus monitored with an egg-hatch assay. Int. J. Parasitol. 27, 1395-1400.
Otsen, M., Hoekstra, R., Plas, M. E., Buntjer, J. B., Lenstra, J. A., Roos, M. H., 2001. Amplified fragment length polymorphism analysis of genetic diversity of Haemonchus contortus during selection for drug resistance. Int. J. Parasitol. 31, 1138-1143.
Ranjan S, Wang G T, Hirschlein C, Simkins K L., 2002. Selection for resistance to macrocyclic lactones by Haemonchus contortus in sheep. Vet Parasitol. 2002 Jan. 3; 103(1-2):109-17.
Roos, M. H., Otsen, M., Hoekstra, R., Veenstra, J. G., Lenstra, J. A., 2004. Genetic analysis of inbreeding of two strains of the parasitic nematode Haemonchus contortus. Int. J. Parasitol. 34, 109-115.
van Wyk, J. A., Malan, F. S., Randles, J. L., 1997. How long before resistance makes it impossible to control some field strains of Haemonchus contortus in South Africa with any of the modern anthelmintics? Vet. Parasitol. 70, 111-122.
Williamson, S. M., Storey, B., Howell, S., Harper, K. M., Kaplan, R. M., Wolstenholme, A. J., 2011. Candidate anthelmintic resistance-associated gene expression and sequence polymorphisms in a triple-resistant field isolate of Haemonchus contortus. Mol. Biochem. Parasitol. 180, 99-105.

What is claimed is:

1. A method for assessing the susceptibility of a nematode to an imidazothiazole anti-helminthic, said method comprising:
(a) providing a genomic DNA sample of the nematode comprising a Hco-acr-8 gene or a Hco-acr-8 gene ortholog;
(b) determining the presence or absence of an indel having the sequence of SEQ ID NO: 9 and being located in the Hco-acr-8 gene or the Hco-acr-8 gene ortholog; and
(c) characterizing the nematode as susceptible to the imidazothiazole anti-helminthic when the indel sequence is determined to be present in the genomic DNA sample and as resistant to the imidazothiazole anti-helminthic when the indel sequence is determined to be absent from the genomic DNA sample.

2. The method of claim 1, wherein the imidazothiazole anti-helminthic is levamisole.

3. The method of claim 1 or 2, wherein the nematode is from a Trichostrongylidae family.

4. The method of claim 3, wherein the nematode is from a Haemonchus genus.

5. The method of claim 4, wherein the nematode is Haemonchus contortus.

6. The method of any one of claims 1 to 5, further comprising providing the genomic DNA from a nematode egg.

7. The method of any one of claims 1 to 6, wherein, prior to step (b), a nucleic-acid synthetic copy of the genomic DNA comprising the Hco-acr-8 gene or the Hco-acr-8 gene ortholog is made and used to determine the presence or absence of the indel.

8. The method of any one of claims 1 to 7, wherein the indel is located between exon 2 and exon 3 of the Hco-acr-8 gene or the Hco-acr-8 gene ortholog.

9. The method of any one of claims 1 to 8, wherein
step (b) further comprises (i) contacting the genomic DNA with at least one pair of primers specific for the vicinity of the indel located in the Hco-acr-8 gene or the Hco-acr-8 gene ortholog under conditions to form a complex between the genomic DNA and the pair of primers, (ii) amplifying the genomic DNA with the at least one pair of primers to provide at least one amplicon, and (iii) determining the presence of the indel in the at least one amplicon; and
step (c) further comprises characterizing the nematode as susceptible to the imidazothiazole anti-helminthic when the indel is determined to be present in the at least one amplicon and as resistant to the imidazothiazole anti-helminthic when the indel is determined to be absent from the at least one amplicon.

10. The method of claim 9, wherein, the at least one pair of primers comprises a first primer and a second primer, wherein the first primer has a nucleic acid sequence corresponding to a first location in the Hco-acr-8g gene or the Hco-acr-8 gene ortholog upstream of the indel and wherein the second primer has a nucleic acid sequence corresponding to a second location in the Hco-acr-8g gene or the Hco-acr-8 gene ortholog downstream of the indel.

11. The method of claim 10, wherein the first primer has a nucleic acid sequence of 5′-ACCTTACCTATACACCCGTC-3′ (SEQ ID NO: 16).

12. The method of claim 10 or 11, wherein the second primer has a nucleic sequence of 5′-CTTGCCGTTATTACACCCTCG-3′ (SEQ ID NO: 17).

13. The method of any one of claims 1 to 8, wherein
step (b) further comprises (i) contacting the genomic DNA with at least a third primer specific for the vicinity of the indel and a fourth primer specific for the indel under conditions to form a complex between the genomic DNA when the indel is present, and (ii) detecting the presence of the complex; and
step (c) further comprises characterizing the nematode as susceptible to the imidazothiazole anti-helminthic when the complex is present and as resistant to the imidazothiazole anti-helminthic when the complex is absent.

14. The method of claim 13, wherein step (b) further comprises (iii) amplifying the genomic DNA with the third primer and the fourth primer to provide at least one amplicon, and (iv) determining the presence of the complex based on the formation of the at least one amplicon.

15. The method of claim 13 or 14, wherein the third primer is specific for a location upstream of the indel.

16. The method of claim 13 or 14, wherein the third primer is specific for a location is downstream of the indel.

17. The method of any one of claims 1 to 8, wherein
step (b) further comprises (i) contacting the genomic DNA with at least one probe specific for the indel located in the Hco-acr-8 gene or the Hco-acr-8 gene ortholog under conditions to form a complex between the genomic DNA and the probe when the indel is present and (ii) detecting the presence of the complex; and
step (c) further comprises characterizing the nematode as susceptible to the imidazothiazole anti-helminthic when the complex is detected and as resistant to the imidazothiazole anti-helminthic when the complex is not detected.

18. The method of claim 17, wherein
step (b) further comprises (i) amplifying the Hco-acr-8 gene, the Hco-acr-8 gene ortholog or a fragment thereof to provide at least one amplicon and (ii) contacting the probe with the at least one amplicon so as to form a complex when the indel is present; and
step (c) further comprises characterizing the nematode as susceptible to the imidazothiazole anti-helminthic when the complex is determined to be present in the at least one amplicon and as resistant to the imidazothiazole anti-helminthic when the complex is determined to be absent from the at least one amplicon.

19. The method of any one of claims 1 to 8, wherein step (b) further comprises extracting an identity of a nucleic acid base at a plurality of positions along the Hco-acr-8 gene, the Hco-acr-8 gene ortholog or a fragment thereof and comparing with the identity of at least one nucleic acid base of the indel at corresponding positions.

20. The method of any one of claims 1 to 19, wherein the nucleic acid residue at position 4 of SEQ ID NO: 9 is T.

21. The method of any one of claims 1 to 20, wherein the nucleic acid residue at position 61 of SEQ ID NO: 9 is C.

22. An isolated nucleic molecule consisting essentially of the nucleic acid sequence of SEQ ID NO: 9.

23. The isolated nucleic acid molecule of claim 22, wherein the nucleic acid residue at position 4 of SEQ ID NO: 9 is T.

24. The isolated nucleic acid molecule of claim 22 or 23, wherein the nucleic acid residue at position 61 of SEQ ID NO: 9 is C.

25. An oligonucleotide having at least 10 consecutives nucleic acid bases of SEQ ID NO: 9.

26. The oligonucleotide of claim 25, being a primer.

27. An oligonucleotide having at least 30 consecutive nucleic acid bases of SEQ ID NO: 9.

28. The oligonucleotide of claim 27, being a probe.

29. A commercial package for the detection of resistance to an imidazothiazole anti-helminthic, said commercial package comprising means for determining the presence or absence of an indel having the sequence of SEQ ID NO: 9 and being located in the a Hco-acr-8 gene or a Hco-acr-8 gene ortholog and instructions for characterizing the resistance of the nematode to the imidazothiazole anti-helminthic based on the presence or absence of the indel.

30. The commercial package of claim 29, wherein the means for determining the presence or absence of the indel comprise a probe specific for the indel.

31. The commercial package of claim 29 or 30, wherein the means for determining the presence or absence of the indel comprise a pair of primers specific for amplifying the indel or a fragment thereof.

32. The commercial package of any one of claims 29 to 31, wherein the nucleic acid residue at position 4 of SEQ ID NO: 9 is T.

33. The commercial package of any one of claims 29 to 32, wherein the nucleic acid residue at position 61 of SEQ ID NO: 9 is C.
